SEQUENCE LISTING (892 sequences)

SEQ ID NO: 1 - protein, 15 aa, synthetic construct
  REGION 1..15: note = Synthetic: Rv1888c
ILLLVAVVALLFTSR

SEQ ID NO: 2 - protein, 15 aa, synthetic construct
  REGION 1..15: note = Synthetic: Rv2095c
EATGLLVALRALADI

SEQ ID NO: 3 - protein, 15 aa, synthetic construct
  REGION 1..15: note = Synthetic: Rv0176
ACLLGLTILLLAVNR

SEQ ID NO: 4 - protein, 15 aa, synthetic construct
  REGION 1..15: note = Synthetic: Rv3874
AEMKTDAATLAQEAG

SEQ ID NO: 5 - protein, 15 aa, synthetic construct
  REGION 1..15: note = Synthetic: Rv3063
AIFMGCYLRFLRPGR

SEQ ID NO: 6 - protein, 15 aa, synthetic construct
  REGION 1..15: note = Synthetic: Rv1351
AQHRHRYLTMVNVGR

SEQ ID NO: 7 - protein, 15 aa, synthetic construct
  REGION 1..15: note = Synthetic: Rv3635
GWLVLIAVLALSLVR

SEQ ID NO: 8 - protein, 15 aa, synthetic construct
  REGION 1..15: note = Synthetic: Rv2051c
ILVGAAAAVVLVAMR

SEQ ID NO: 9 - protein, 15 aa, synthetic construct
  REGION 1..15: note = Synthetic: Rv0176
IRRLTAAVALAAAGA

SEQ ID NO: 10 - protein, 15 aa, synthetic construct
  REGION 1..15: note = Synthetic: Rv1508c
IVVVAVLALGRRIPP

SEQ ID NO: 11 - protein, 15 aa, synthetic construct
  REGION 1..15: note = Synthetic: Rv1349
LIGFALLAFCSVVAR

SEQ ID NO: 12 - protein, 15 aa, synthetic construct
  REGION 1..15: note = Synthetic: Rv3645
TVIILFLAGAVVNLK

SEQ ID NO: 13 - protein, 15 aa, synthetic construct
  REGION 1..15: note = Synthetic: Rv1807
AAAAAYEAAFAATVP

SEQ ID NO: 14 - protein, 15 aa, synthetic construct
  REGION 1..15: note = Synthetic: Rv2338c
AAEVLKILLGHGRVY

SEQ ID NO: 15 - protein, 15 aa, synthetic construct
  REGION 1..15: note = Synthetic: Rv1195
AAIHDQFVATLASSA

SEQ ID NO: 16 - protein, 15 aa, synthetic construct
  REGION 1..15: note = Synthetic: Rv3125c
AAIQARAAATAFEAA

SEQ ID NO: 17 - protein, 15 aa, synthetic construct
  REGION 1..15: note = Synthetic: Rv0205
AAVLVLFTLIFFLYG

SEQ ID NO: 18 - protein, 15 aa, synthetic construct
  REGION 1..15: note = Synthetic: Rv3261
ADADIIMLAPSNPVV

SEQ ID NO: 19 - protein, 15 aa, synthetic construct
  REGION 1..15: note = Synthetic: Rv0144
ADAQYPASTAFLATT

SEQ ID NO: 20 - protein, 15 aa, synthetic construct
  REGION 1..15: note = Synthetic: Rv0016c
ADGLRADPRNQRVLL

SEQ ID NO: 21 - protein, 15 aa, synthetic construct
  REGION 1..15: note = Synthetic: Rv1807
AGAQAEGAAAAYEAA

SEQ ID NO: 22 - protein, 15 aa, synthetic construct
  REGION 1..15: note = Synthetic: Rv0590A
AGGIYGDFFNFYLCD

SEQ ID NO: 23 - protein, 15 aa, synthetic construct
  REGION 1..15: note = Synthetic: Rv0284
AGHTHYLIIDDVDQV

SEQ ID NO: 24 - protein, 15 aa, synthetic construct
  REGION 1..15: note = Synthetic: Rv2040c
AILLVLTVLQLRITH

SEQ ID NO: 25 - protein, 15 aa, synthetic construct
  REGION 1..15: note = Synthetic: Rv0676c
ALCLIFIIMLIITRS

SEQ ID NO: 26 - protein, 15 aa, synthetic construct
  REGION 1..15: note = Synthetic: Rv0676c
ALCLIFIIMLITTRS

SEQ ID NO: 27 - protein, 15 aa, synthetic construct
  REGION 1..15: note = Synthetic: Rv1997
ANRWIILGVSAQAIA

SEQ ID NO: 28 - protein, 15 aa, synthetic construct
  REGION 1..15: note = Synthetic: Rv1227c
APVMWALSASLGWIL

SEQ ID NO: 29 - protein, 15 aa, synthetic construct
  REGION 1..15: note = Synthetic: Rv3782
AQEWLRFGWFFLATR

SEQ ID NO: 30 - protein, 15 aa, synthetic construct
  REGION 1..15: note = Synthetic: Rv3782
AQEWLRFGWFFLVTR

SEQ ID NO: 31 - protein, 15 aa, synthetic construct
  REGION 1..15: note = Synthetic: Rv3071
ARFFGRNVNVPLMII

SEQ ID NO: 32 - protein, 15 aa, synthetic construct
  REGION 1..15: note = Synthetic: Rv2024c
ARILLLVPSISLLSQ

SEQ ID NO: 33 - protein, 15 aa, synthetic construct
  REGION 1..15: note = Synthetic: Rv3763
ASGPKVVIDGKDQNV

SEQ ID NO: 34 - protein, 15 aa, synthetic construct
  REGION 1..15: note = Synthetic: Rv3887c
ATRFRPIIRLTVEWL

SEQ ID NO: 35 - protein, 15 aa, synthetic construct
  REGION 1..15: note = Synthetic: Rv3887c
ATRVRPIIRLTVEWL

SEQ ID NO: 36 - protein, 15 aa, synthetic construct
  REGION 1..15: note = Synthetic: Rv1894c
AVGFTPEQLEIELNW

SEQ ID NO: 37 - protein, 15 aa, synthetic construct
  REGION 1..15: note = Synthetic: Rv3436c
AVLRRLEWHFTLVFA

SEQ ID NO: 38 - protein, 15 aa, synthetic construct
  REGION 1..15: note = Synthetic: Rv1886c
DIKVQFQSGGNNSPA

SEQ ID NO: 39 - protein, 15 aa, synthetic construct
  REGION 1..15: note = Synthetic: Rv3432c
DLVWDFRLPRVVSIN

SEQ ID NO: 40 - protein, 15 aa, synthetic construct
  REGION 1..15: note = Synthetic: Rv0107c
DQLVCRVVVPAVATT

SEQ ID NO: 41 - protein, 15 aa, synthetic construct
  REGION 1..15: note = Synthetic: Rv0107c
DQLVRRVVVPAVATT

SEQ ID NO: 42 - protein, 15 aa, synthetic construct
  REGION 1..15: note = Synthetic: Rv3784
DRYINWAKDQPQYPY

SEQ ID NO: 43 - protein, 15 aa, synthetic construct
  REGION 1..15: note = Synthetic: Rv0040c
DSQKLLAWQTTNASM

SEQ ID NO: 44 - protein, 15 aa, synthetic construct
  REGION 1..15: note = Synthetic: Rv2095c
EATGLLVALRALANI

SEQ ID NO: 45 - protein, 15 aa, synthetic construct
  REGION 1..15: note = Synthetic: Rv3646c
EDALRLLSLPRVVGV

SEQ ID NO: 46 - protein, 15 aa, synthetic construct
  REGION 1..15: note = Synthetic: Rv0440
EDAVRNAKAAVEEGI

SEQ ID NO: 47 - protein, 15 aa, synthetic construct
  REGION 1..15: note = Synthetic: Rv0288
EDLVRAYHAMSRTHE

SEQ ID NO: 48 - protein, 15 aa, synthetic construct
  REGION 1..15: note = Synthetic: Rv0361
EDQVRQAIQSLDIAI

SEQ ID NO: 49 - protein, 15 aa, synthetic construct
  REGION 1..15: note = Synthetic: Rv0876c
ELWVGFTAVSALLIL

SEQ ID NO: 50 - protein, 15 aa, synthetic construct
  REGION 1..15: note = Synthetic: Rv0573c
EQDLRYLRGLGQFSD

SEQ ID NO: 51 - protein, 15 aa, synthetic construct
  REGION 1..15: note = Synthetic: Rv2096c
ERLVNLVIALLSTRG

SEQ ID NO: 52 - protein, 15 aa, synthetic construct
  REGION 1..15: note = Synthetic: Rv2032
ESLRLYDSSYHAELF

SEQ ID NO: 53 - protein, 15 aa, synthetic construct
  REGION 1..15: note = Synthetic: Rv0276
ESPVSYHAFDPELQL

SEQ ID NO: 54 - protein, 15 aa, synthetic construct
  REGION 1..15: note = Synthetic: Rv0284
EVFLVIDNLYGFGRD

SEQ ID NO: 55 - protein, 15 aa, synthetic construct
  REGION 1..15: note = Synthetic: Rv3794
FCLFGVLFVLLRRGR

SEQ ID NO: 56 - protein, 15 aa, synthetic construct
  REGION 1..15: note = Synthetic: Rv0284
FFPFVLLLAATALYR

SEQ ID NO: 57 - protein, 15 aa, synthetic construct
  REGION 1..15: note = Synthetic: Rv0731c
FGEVIYVSAELKQKH

SEQ ID NO: 58 - protein, 15 aa, synthetic construct
  REGION 1..15: note = Synthetic: Rv0276
FNLMLWDLDRRMRRG

SEQ ID NO: 59 - protein, 15 aa, synthetic construct
  REGION 1..15: note = Synthetic: Rv1894c
FPIFAVTHCRDVVVA

SEQ ID NO: 60 - protein, 15 aa, synthetic construct
  REGION 1..15: note = Synthetic: Rv1629
FRAFYALPAENFKTR

SEQ ID NO: 61 - protein, 15 aa, synthetic construct
  REGION 1..15: note = Synthetic: Rv1807
GAAAAYEAAFAATVP

SEQ ID NO: 62 - protein, 15 aa, synthetic construct
  REGION 1..15: note = Synthetic: Rv2346c
GAMIRAQAGLLEAEH

SEQ ID NO: 63 - protein, 15 aa, synthetic construct
  REGION 1..15: note = Synthetic: Rv0160c
GGLLARFPGFYIPFL

SEQ ID NO: 64 - protein, 15 aa, synthetic construct
  REGION 1..15: note = Synthetic: Rv3493c
GIDEISDNASVLVSV

SEQ ID NO: 65 - protein, 15 aa, synthetic construct
  REGION 1..15: note = Synthetic: Rv1173
GIGRPARQRTTTYAL

SEQ ID NO: 66 - protein, 15 aa, synthetic construct
  REGION 1..15: note = Synthetic: Rv0284
GILIVGMIVALVATG

SEQ ID NO: 67 - protein, 15 aa, synthetic construct
  REGION 1..15: note = Synthetic: Rv0694
GITVADNVAAFSELG

SEQ ID NO: 68 - protein, 15 aa, synthetic construct
  REGION 1..15: note = Synthetic: Rv3910
GLSSVFMAILNTRNM

SEQ ID NO: 69 - protein, 15 aa, synthetic construct
  REGION 1..15: note = Synthetic: Rv3910
GLSSVFMAILNTRNV

SEQ ID NO: 70 - protein, 15 aa, synthetic construct
  REGION 1..15: note = Synthetic: Rv0017c
GLTAILMLYSIVIIR

SEQ ID NO: 71 - protein, 15 aa, synthetic construct
  REGION 1..15: note = Synthetic: Rv1890c
GNGYRLTGKLHIRGK

SEQ ID NO: 72 - protein, 15 aa, synthetic construct
  REGION 1..15: note = Synthetic: Rv3252c
GPILLYVLLPLLDLR

SEQ ID NO: 73 - protein, 15 aa, synthetic construct
  REGION 1..15: note = Synthetic: Rv3818
GSTADFTGTTLNSLR

SEQ ID NO: 74 - protein, 15 aa, synthetic construct
  REGION 1..15: note = Synthetic: Rv1122
GSWLLDLTAIALRES

SEQ ID NO: 75 - protein, 15 aa, synthetic construct
  REGION 1..15: note = Synthetic: Rv1888c
ILLLVAVVALLFTGR

SEQ ID NO: 76 - protein, 15 aa, synthetic construct
  REGION 1..15: note = Synthetic: Rv3383c
ILLRDALHATAVRIL

SEQ ID NO: 77 - protein, 15 aa, synthetic construct
  REGION 1..15: note = Synthetic: Rv1681
IRLALSLGFRVRVAA

SEQ ID NO: 78 - protein, 15 aa, synthetic construct
  REGION 1..15: note = Synthetic: Rv1565c
IRRLLPALVVVLAGC

SEQ ID NO: 79 - protein, 15 aa, synthetic construct
  REGION 1..15: note = Synthetic: Rv0268c
ISAERYESLMEELED

SEQ ID NO: 80 - protein, 15 aa, synthetic construct
  REGION 1..15: note = Synthetic: Rv0284
ISPQTLFFPFVLLLA

SEQ ID NO: 81 - protein, 15 aa, synthetic construct
  REGION 1..15: note = Synthetic: Rv3796
IVDYYNFFLSGGFLA

SEQ ID NO: 82 - protein, 15 aa, synthetic construct
  REGION 1..15: note = Synthetic: Rv1813c
KAWHQRTPARAEQVA

SEQ ID NO: 83 - protein, 15 aa, synthetic construct
  REGION 1..15: note = Synthetic: Rv2024c
KDDIFYYVYGLLHDP

SEQ ID NO: 84 - protein, 15 aa, synthetic construct
  REGION 1..15: note = Synthetic: Rv1442
KTPEWAAALSGLAAG

SEQ ID NO: 85 - protein, 15 aa, synthetic construct
  REGION 1..15: note = Synthetic: Rv1111c
LGTAAGVLLIGLVRW

SEQ ID NO: 86 - protein, 15 aa, synthetic construct
  REGION 1..15: note = Synthetic: Rv3796
LHRAFAYTSNIFIRD

SEQ ID NO: 87 - protein, 15 aa, synthetic construct
  REGION 1..15: note = Synthetic: Rv0284
LIIDDVDQVPDSPAM

SEQ ID NO: 88 - protein, 15 aa, synthetic construct
  REGION 1..15: note = Synthetic: Rv0174
LILFAIVSVVAIVVL

SEQ ID NO: 89 - protein, 15 aa, synthetic construct
  REGION 1..15: note = Synthetic: Rv0585c
LPFVILIGLIVSRQW

SEQ ID NO: 90 - protein, 15 aa, synthetic construct
  REGION 1..15: note = Synthetic: Rv0290
LPLRRLLGLVAAGLD

SEQ ID NO: 91 - protein, 15 aa, synthetic construct
  REGION 1..15: note = Synthetic: Rv1599
LPTVRPIVAAVAERG

SEQ ID NO: 92 - protein, 15 aa, synthetic construct
  REGION 1..15: note = Synthetic: Rv3365c
LRRNSANLLVLAGAQ

SEQ ID NO: 93 - protein, 15 aa, synthetic construct
  REGION 1..15: note = Synthetic: Rv0267
LSRAGLFFVPLAVAA

SEQ ID NO: 94 - protein, 15 aa, synthetic construct
  REGION 1..15: note = Synthetic: Rv3557c
LVYRFIRDTTWVSVR

SEQ ID NO: 95 - protein, 15 aa, synthetic construct
  REGION 1..15: note = Synthetic: Rv3522
MLMPCFAQLYDELGI

SEQ ID NO: 96 - protein, 15 aa, synthetic construct
  REGION 1..15: note = Synthetic: Rv3782
NRGYVLSQPGLRKLL

SEQ ID NO: 97 - protein, 15 aa, synthetic construct
  REGION 1..15: note = Synthetic: Rv3458c
NVPSYRVSQYDIVDV

SEQ ID NO: 98 - protein, 15 aa, synthetic construct
  REGION 1..15: note = Synthetic: Rv0244c
NVRDQVFNLFEVLGV

SEQ ID NO: 99 - protein, 15 aa, synthetic construct
  REGION 1..15: note = Synthetic: Rv3818
PDWVNTIFLSTRFRA

SEQ ID NO: 100 - protein, 15 aa, synthetic construct
  REGION 1..15: note = Synthetic: Rv0235c
QLYQYRFTTVAELRR

SEQ ID NO: 101 - protein, 15 aa, synthetic construct
  REGION 1..15: note = Synthetic: Rv0290
QSGFIAAAVLLSVLG

SEQ ID NO: 102 - protein, 15 aa, synthetic construct
  REGION 1..15: note = Synthetic: Rv0836c
RATFRALGSTGHRFL

SEQ ID NO: 103 - protein, 15 aa, synthetic construct
  REGION 1..15: note = Synthetic: Rv3182
RILFAFDPARQAILL

SEQ ID NO: 104 - protein, 15 aa, synthetic construct
  REGION 1..15: note = Synthetic: Rv3125c
SMLFLPVRILTSPIT

SEQ ID NO: 105 - protein, 15 aa, synthetic construct
  REGION 1..15: note = Synthetic: Rv2095c
SPPGLPVAAVAEQAP

SEQ ID NO: 106 - protein, 15 aa, synthetic construct
  REGION 1..15: note = Synthetic: Rv1196
SSAGLMVAAASPYVA

SEQ ID NO: 107 - protein, 15 aa, synthetic construct
  REGION 1..15: note = Synthetic: Rv3635
SSWRPVLLIPLTFAL

SEQ ID NO: 108 - protein, 15 aa, synthetic construct
  REGION 1..15: note = Synthetic: Rv1196
TATATLLPFEEAPEM

SEQ ID NO: 109 - protein, 15 aa, synthetic construct
  REGION 1..15: note = Synthetic: Rv1005c
TTPSVLFLLLKTIAT

SEQ ID NO: 110 - protein, 15 aa, synthetic construct
  REGION 1..15: note = Synthetic: Rv3148
VLMMELNRISSHLVA

SEQ ID NO: 111 - protein, 15 aa, synthetic construct
  REGION 1..15: note = Synthetic: Rv1565c
WPLLIFWLSYTGHRH

SEQ ID NO: 112 - protein, 15 aa, synthetic construct
  REGION 1..15: note = Synthetic: Rv0266c
YLRIAVANMANAVKK

SEQ ID NO: 113 - protein, 15 aa, synthetic construct
  REGION 1..15: note = Synthetic: Rv0276
YLYPIVALRIRGIAL

SEQ ID NO: 114 - protein, 15 aa, synthetic construct
  REGION 1..15: note = Synthetic: Rv0289
DRWLDLRYVGPASAD

SEQ ID NO: 115 - protein, 15 aa, synthetic construct
  REGION 1..15: note = Synthetic: Rv3330
LENDNQLLYNYPGAL

SEQ ID NO: 116 - protein, 15 aa, synthetic construct
  REGION 1..15: note = Synthetic: Rv3874
AAVVRFQEAANKQKQ

SEQ ID NO: 117 - protein, 15 aa, synthetic construct
  REGION 1..15: note = Synthetic: Rv3024c
QQIKFAALSARAVAL

SEQ ID NO: 118 - protein, 15 aa, synthetic construct
  REGION 1..15: note = Synthetic: Rv2123
ADYLRMWIQAATVMS

SEQ ID NO: 119 - protein, 15 aa, synthetic construct
  REGION 1..15: note = Synthetic: Rv3875
EQQWNFAGIEAAASA

SEQ ID NO: 120 - protein, 15 aa, synthetic construct
  REGION 1..15: note = Synthetic: Rv3418c
GEEYLILSARDVLAV

SEQ ID NO: 121 - protein, 15 aa, synthetic construct
  REGION 1..15: note = Synthetic: Rv0290
AAGVAAWSLIALMIP

SEQ ID NO: 122 - protein, 15 aa, synthetic construct
  REGION 1..15: note = Synthetic: Rv2874
AALPLLFFALAGQRI

SEQ ID NO: 123 - protein, 15 aa, synthetic construct
  REGION 1..15: note = Synthetic: Rv3804c
AGCQTYKWETFLTSE

SEQ ID NO: 124 - protein, 15 aa, synthetic construct
  REGION 1..15: note = Synthetic: Rv2875
ANATVYMIDSVLMPP

SEQ ID NO: 125 - protein, 15 aa, synthetic construct
  REGION 1..15: note = Synthetic: Rv3018c
DYVRMWVQAATVMSA

SEQ ID NO: 126 - protein, 15 aa, synthetic construct
  REGION 1..15: note = Synthetic: Rv3022c
ELFVAAYVPYVAWLV

SEQ ID NO: 127 - protein, 15 aa, synthetic construct
  REGION 1..15: note = Synthetic: Rv0129c
GQNYTYKWETFLTRE

SEQ ID NO: 128 - protein, 15 aa, synthetic construct
  REGION 1..15: note = Synthetic: Rv3876
RQSGATIADVLAEKE

SEQ ID NO: 129 - protein, 15 aa, synthetic construct
  REGION 1..15: note = Synthetic: Rv3015c
YRIAARPGAVTRRAA

SEQ ID NO: 130 - protein, 15 aa, synthetic construct
  REGION 1..15: note = Synthetic: Rv0290
AAGAQLLWQLPLLSI

SEQ ID NO: 131 - protein, 15 aa, synthetic construct
  REGION 1..15: note = Synthetic: Rv1791
AAIHEMFVNTLVASS

SEQ ID NO: 132 - protein, 15 aa, synthetic construct
  REGION 1..15: note = Synthetic: Rv3021c
AAVPAVGAAAGAPAA

SEQ ID NO: 133 - protein, 15 aa, synthetic construct
  REGION 1..15: note = Synthetic: Rv3115
AGWLAFFRDLVARGL

SEQ ID NO: 134 - protein, 15 aa, synthetic construct
  REGION 1..15: note = Synthetic: Rv3024c
AHGETVSAVAELIGD

SEQ ID NO: 135 - protein, 15 aa, synthetic construct
  REGION 1..15: note = Synthetic: Rv0291
ALSVLVGLTAATVAI

SEQ ID NO: 136 - protein, 15 aa, synthetic construct
  REGION 1..15: note = Synthetic: Rv3012c
AVDGRFAVPQILGDE

SEQ ID NO: 137 - protein, 15 aa, synthetic construct
  REGION 1..15: note = Synthetic: Rv0292
EIGWEAGTAAPDEIP

SEQ ID NO: 138 - protein, 15 aa, synthetic construct
  REGION 1..15: note = Synthetic: Rv3018c
EIVQFLEETFAAYDQ

SEQ ID NO: 139 - protein, 15 aa, synthetic construct
  REGION 1..15: note = Synthetic: Rv0289
GNGVVALRNAQLVTF

SEQ ID NO: 140 - protein, 15 aa, synthetic construct
  REGION 1..15: note = Synthetic: Rv2874
GTVVLTATFALGAAL

SEQ ID NO: 141 - protein, 15 aa, synthetic construct
  REGION 1..15: note = Synthetic: Rv0293c
LNYRPLLPKDRRMII

SEQ ID NO: 142 - protein, 15 aa, synthetic construct
  REGION 1..15: note = Synthetic: Rv3115
LRGLLSTFIAALMGA

SEQ ID NO: 143 - protein, 15 aa, synthetic construct
  REGION 1..15: note = Synthetic: Rv1195
MHVSFVMAYPEMLAA

SEQ ID NO: 144 - protein, 15 aa, synthetic construct
  REGION 1..15: note = Synthetic: Rv0299
RVPEDLLAMVVAVEQ

SEQ ID NO: 145 - protein, 15 aa, synthetic construct
  REGION 1..15: note = Synthetic: Rv3115
YLGLEVLTRARAALT

SEQ ID NO: 146 - protein, 15 aa, synthetic construct
  REGION 1..15: note = Synthetic: Rv1802
YVAWMSATAALAREA

SEQ ID NO: 147 - protein, 15 aa, synthetic construct
  REGION 1..15: note = Synthetic: Rv0291
AARLLSIRAMSTKFS

SEQ ID NO: 148 - protein, 15 aa, synthetic construct
  REGION 1..15: note = Synthetic: Rv0294
AKLMRDIPFRVGAVV

SEQ ID NO: 149 - protein, 15 aa, synthetic construct
  REGION 1..15: note = Synthetic: Rv0292
ALTALIRDPPADSTG

SEQ ID NO: 150 - protein, 15 aa, synthetic construct
  REGION 1..15: note = Synthetic: Rv1808
AQLSQLISLLPSTLQ

SEQ ID NO: 151 - protein, 15 aa, synthetic construct
  REGION 1..15: note = Synthetic: Rv0256c
ARMWIQAATTMASYQ

SEQ ID NO: 152 - protein, 15 aa, synthetic construct
  REGION 1..15: note = Synthetic: Rv3018c
DYVRMWVQAATAMSA

SEQ ID NO: 153 - protein, 15 aa, synthetic construct
  REGION 1..15: note = Synthetic: Rv0289
EVVDYLGIPASARPV

SEQ ID NO: 154 - protein, 15 aa, synthetic construct
  REGION 1..15: note = Synthetic: Rv2024c
GADATAAAAFEQFLA

SEQ ID NO: 155 - protein, 15 aa, synthetic construct
  REGION 1..15: note = Synthetic: Rv0453
GAMVATNFFGINTIP

SEQ ID NO: 156 - protein, 15 aa, synthetic construct
  REGION 1..15: note = Synthetic: Rv3021c
GWIISNIFGAIPVLG

SEQ ID NO: 157 - protein, 15 aa, synthetic construct
  REGION 1..15: note = Synthetic: Rv0985c
IDLNVLLSAAINFFL

SEQ ID NO: 158 - protein, 15 aa, synthetic construct
  REGION 1..15: note = Synthetic: Rv3025c
ILPIAEMSVVAMEFG

SEQ ID NO: 159 - protein, 15 aa, synthetic construct
  REGION 1..15: note = Synthetic: Rv1808
LAAAAAWDALAAELY

SEQ ID NO: 160 - protein, 15 aa, synthetic construct
  REGION 1..15: note = Synthetic: Rv2874
LALVGFLGGLITGIS

SEQ ID NO: 161 - protein, 15 aa, synthetic construct
  REGION 1..15: note = Synthetic: Rv2874
LALVGFLGGLITGTS

SEQ ID NO: 162 - protein, 15 aa, synthetic construct
  REGION 1..15: note = Synthetic: Rv1366
LQSLWANFYELLADA

SEQ ID NO: 163 - protein, 15 aa, synthetic construct
  REGION 1..15: note = Synthetic: Rv1196
LSPISNMVSMANNHM

SEQ ID NO: 164 - protein, 15 aa, synthetic construct
  REGION 1..15: note = Synthetic: Rv0294
MWDPDVYLAFSGHRN

SEQ ID NO: 165 - protein, 15 aa, synthetic construct
  REGION 1..15: note = Synthetic: Rv0690c
MYRELLELVAADVES

SEQ ID NO: 166 - protein, 15 aa, synthetic construct
  REGION 1..15: note = Synthetic: Rv3115
QASPDLLRGLLSTFI

SEQ ID NO: 167 - protein, 15 aa, synthetic construct
  REGION 1..15: note = Synthetic: Rv0293c
RCALHWFPGSHLLAC

SEQ ID NO: 168 - protein, 15 aa, synthetic construct
  REGION 1..15: note = Synthetic: Rv0298
VTVDAAVLAAIDADA

SEQ ID NO: 169 - protein, 15 aa, synthetic construct
  REGION 1..15: note = Synthetic: Rv1788
AAIHEMFVNTLQMSS

SEQ ID NO: 170 - protein, 15 aa, synthetic construct
  REGION 1..15: note = Synthetic: Rv3021c
AALPAVGAAAGAPAA

SEQ ID NO: 171 - protein, 15 aa, synthetic construct
  REGION 1..15: note = Synthetic: Rv1198
AEHQAIVRDVLAAGD

SEQ ID NO: 172 - protein, 15 aa, synthetic construct
  REGION 1..15: note = Synthetic: Rv1198
AEHQAIVRDVLAASD

SEQ ID NO: 173 - protein, 15 aa, synthetic construct
  REGION 1..15: note = Synthetic: Rv3024c
AEKFKEDVINDFVSS

SEQ ID NO: 174 - protein, 15 aa, synthetic construct
  REGION 1..15: note = Synthetic: Rv1361c
AELMILIATNLLGQN

SEQ ID NO: 175 - protein, 15 aa, synthetic construct
  REGION 1..15: note = Synthetic: Rv1172c
ALLPRAGAAAAAALP

SEQ ID NO: 176 - protein, 15 aa, synthetic construct
  REGION 1..15: note = Synthetic: Rv3025c
ALQSHDDVALVSVMW

SEQ ID NO: 177 - protein, 15 aa, synthetic construct
  REGION 1..15: note = Synthetic: Rv3022c
ALSAEYAAVAQELSV

SEQ ID NO: 178 - protein, 15 aa, synthetic construct
  REGION 1..15: note = Synthetic: Rv1172c
ALSRVHSMFLGTGGS

SEQ ID NO: 179 - protein, 15 aa, synthetic construct
  REGION 1..15: note = Synthetic: Rv1788
AQIYQAVSAQAAAIH

SEQ ID NO: 180 - protein, 15 aa, synthetic construct
  REGION 1..15: note = Synthetic: Rv3115
ASIIRLVGAVLAEQH

SEQ ID NO: 181 - protein, 15 aa, synthetic construct
  REGION 1..15: note = Synthetic: Rv1366
AVHVWLRLPAGRVEI

SEQ ID NO: 182 - protein, 15 aa, synthetic construct
  REGION 1..15: note = Synthetic: Rv0690c
AVPLRLLGGLHRMVL

SEQ ID NO: 183 - protein, 15 aa, synthetic construct
  REGION 1..15: note = Synthetic: Rv0298
AYAQRVYQANRAAGS

SEQ ID NO: 184 - protein, 15 aa, synthetic construct
  REGION 1..15: note = Synthetic: Rv0294
DESWQQFRQELIPLL

SEQ ID NO: 185 - protein, 15 aa, synthetic construct
  REGION 1..15: note = Synthetic: Rv1705c
FGQNTASIAATEAQY

SEQ ID NO: 186 - protein, 15 aa, synthetic construct
  REGION 1..15: note = Synthetic: Rv1802
FGQNTGAIAAAEARY

SEQ ID NO: 187 - protein, 15 aa, synthetic construct
  REGION 1..15: note = Synthetic: Rv1789
FGQNTSAIAAAEAQY

SEQ ID NO: 188 - protein, 15 aa, synthetic construct
  REGION 1..15: note = Synthetic: Rv2853
FVQALTTAAASYASV

SEQ ID NO: 189 - protein, 15 aa, synthetic construct
  REGION 1..15: note = Synthetic: Rv3021c
GWIISNIFGAIPVLA

SEQ ID NO: 190 - protein, 15 aa, synthetic construct
  REGION 1..15: note = Synthetic: Rv0987
IGLVTQTINDFYFVI

SEQ ID NO: 191 - protein, 15 aa, synthetic construct
  REGION 1..15: note = Synthetic: Rv2996c
INLIIHYVHRAGALG

SEQ ID NO: 192 - protein, 15 aa, synthetic construct
  REGION 1..15: note = Synthetic: Rv3136
IQARAAALAFEQAYA

SEQ ID NO: 193 - protein, 15 aa, synthetic construct
  REGION 1..15: note = Synthetic: Rv1317c
IRQLERLLQAVVGAG

SEQ ID NO: 194 - protein, 15 aa, synthetic construct
  REGION 1..15: note = Synthetic: Rv1196
LGGLWTAVSPHLSPL

SEQ ID NO: 195 - protein, 15 aa, synthetic construct
  REGION 1..15: note = Synthetic: Rv1196
LSPISNMVSMANNHV

SEQ ID NO: 196 - protein, 15 aa, synthetic construct
  REGION 1..15: note = Synthetic: Rv1196
LSPLSNMVSMANNHM

SEQ ID NO: 197 - protein, 15 aa, synthetic construct
  REGION 1..15: note = Synthetic: Rv3804c
PSPSMGRDIKVQFQS

SEQ ID NO: 198 - protein, 15 aa, synthetic construct
  REGION 1..15: note = Synthetic: Rv2031c
QMATTLPVQRHPRSL

SEQ ID NO: 199 - protein, 15 aa, synthetic construct
  REGION 1..15: note = Synthetic: Rv0129c
QVPSASMGRDIKVQF

SEQ ID NO: 200 - protein, 15 aa, synthetic construct
  REGION 1..15: note = Synthetic: Rv0293c
RCALHWFPGSHLLHV

SEQ ID NO: 201 - protein, 15 aa, synthetic construct
  REGION 1..15: note = Synthetic: Rv1196
RSPISNMVSMANNHM

SEQ ID NO: 202 - protein, 15 aa, synthetic construct
  REGION 1..15: note = Synthetic: Rv3025c
SARLRLLRDRLVEGV

SEQ ID NO: 203 - protein, 15 aa, synthetic construct
  REGION 1..15: note = Synthetic: Rv0294
STIFPFRRLFMVADV

SEQ ID NO: 204 - protein, 15 aa, synthetic construct
  REGION 1..15: note = Synthetic: Rv1802
AAAQASAAAAAYEAA

SEQ ID NO: 205 - protein, 15 aa, synthetic construct
  REGION 1..15: note = Synthetic: Rv2823c
AAFSRMLSLFFRQHI

SEQ ID NO: 206 - protein, 15 aa, synthetic construct
  REGION 1..15: note = Synthetic: Rv3015c
AASLLDEDMDALEEA

SEQ ID NO: 207 - protein, 15 aa, synthetic construct
  REGION 1..15: note = Synthetic: Rv1789
AATQARAAAAAFEAA

SEQ ID NO: 208 - protein, 15 aa, synthetic construct
  REGION 1..15: note = Synthetic: Rv0291
AAVDKDAVIVAAAGN

SEQ ID NO: 209 - protein, 15 aa, synthetic construct
  REGION 1..15: note = Synthetic: Rv0256c
AEAPAAAAAPEEQVQ

SEQ ID NO: 210 - protein, 15 aa, synthetic construct
  REGION 1..15: note = Synthetic: Rv3619c
AEHQAIISDVLTASD

SEQ ID NO: 211 - protein, 15 aa, synthetic construct
  REGION 1..15: note = Synthetic: Rv0292
AILRRRRRIAEPATC

SEQ ID NO: 212 - protein, 15 aa, synthetic construct
  REGION 1..15: note = Synthetic: Rv1172c
ALSRVQSMFLGTGGS

SEQ ID NO: 213 - protein, 15 aa, synthetic construct
  REGION 1..15: note = Synthetic: Rv1172c
APQINFFYYLGEPIV

SEQ ID NO: 214 - protein, 15 aa, synthetic construct
  REGION 1..15: note = Synthetic: Rv1705c
APYVAWMRATAIQAE

SEQ ID NO: 215 - protein, 15 aa, synthetic construct
  REGION 1..15: note = Synthetic: Rv0293c
AQAVYDFRSIVDYLR

SEQ ID NO: 216 - protein, 15 aa, synthetic construct
  REGION 1..15: note = Synthetic: Rv1317c
AQLGYTIRQLERLLQ

SEQ ID NO: 217 - protein, 15 aa, synthetic construct
  REGION 1..15: note = Synthetic: Rv1196
AQNGVQAMSSLGSSL

SEQ ID NO: 218 - protein, 15 aa, synthetic construct
  REGION 1..15: note = Synthetic: Rv2024c
ARILRQLATPISVII

SEQ ID NO: 219 - protein, 15 aa, synthetic construct
  REGION 1..15: note = Synthetic: Rv3115
ARTDLLAFTAFPKQI

SEQ ID NO: 220 - protein, 15 aa, synthetic construct
  REGION 1..15: note = Synthetic: Rv0291
ATEVVRRLTATAHRG

SEQ ID NO: 221 - protein, 15 aa, synthetic construct
  REGION 1..15: note = Synthetic: Rv1789
AWMSAAAAQAEQAAT

SEQ ID NO: 222 - protein, 15 aa, synthetic construct
  REGION 1..15: note = Synthetic: Rv0987
CILAWILVRIINVRS

SEQ ID NO: 223 - protein, 15 aa, synthetic construct
  REGION 1..15: note = Synthetic: Rv0129c
DPMVQIPRLVANNTR

SEQ ID NO: 224 - protein, 15 aa, synthetic construct
  REGION 1..15: note = Synthetic: Rv0299
EHELYVAVLSNALHR

SEQ ID NO: 225 - protein, 15 aa, synthetic construct
  REGION 1..15: note = Synthetic: Rv2823c
FDHEFTFGWDELLSK

SEQ ID NO: 226 - protein, 15 aa, synthetic construct
  REGION 1..15: note = Synthetic: Rv2823c
FDREFTFGWDELLSK

SEQ ID NO: 227 - protein, 15 aa, synthetic construct
  REGION 1..15: note = Synthetic: Rv1199c
FPDRASIIRLVGAVL

SEQ ID NO: 228 - protein, 15 aa, synthetic construct
  REGION 1..15: note = Synthetic: Rv2823c
FYNEKAFLLTTFDVS

SEQ ID NO: 229 - protein, 15 aa, synthetic construct
  REGION 1..15: note = Synthetic: Rv0453
GWSSLGREYAAVAEE

SEQ ID NO: 230 - protein, 15 aa, synthetic construct
  REGION 1..15: note = Synthetic: Rv1886c
HPQQFIYAGSLSALL

SEQ ID NO: 231 - protein, 15 aa, synthetic construct
  REGION 1..15: note = Synthetic: Rv2024c
IMLLAYYIAAVNIES

SEQ ID NO: 232 - protein, 15 aa, synthetic construct
  REGION 1..15: note = Synthetic: Rv2996c
INLIIHYVDRPGALG

SEQ ID NO: 233 - protein, 15 aa, synthetic construct
  REGION 1..15: note = Synthetic: Rv2996c
IVQINGRHFDLRAQG

SEQ ID NO: 234 - protein, 15 aa, synthetic construct
  REGION 1..15: note = Synthetic: Rv0256c
LAWLVQASANSAAMA

SEQ ID NO: 235 - protein, 15 aa, synthetic construct
  REGION 1..15: note = Synthetic: Rv0293c
LDYLRRMTVFLQGLM

SEQ ID NO: 236 - protein, 15 aa, synthetic construct
  REGION 1..15: note = Synthetic: Rv3021c
LLEFAVVLELAILSI

SEQ ID NO: 237 - protein, 15 aa, synthetic construct
  REGION 1..15: note = Synthetic: Rv3019c
LQSLGADIASEQAVL

SEQ ID NO: 238 - protein, 15 aa, synthetic construct
  REGION 1..15: note = Synthetic: Rv3330
MAFLRSVSCLAAAVF

SEQ ID NO: 239 - protein, 15 aa, synthetic construct
  REGION 1..15: note = Synthetic: Rv3330
MAFLRSVSRLAAAVF

SEQ ID NO: 240 - protein, 15 aa, synthetic construct
  REGION 1..15: note = Synthetic: Rv1788
MSFVTTQPEALAAAA

SEQ ID NO: 241 - protein, 15 aa, synthetic construct
  REGION 1..15: note = Synthetic: Rv1789
NRASLMQLISTNVFG

SEQ ID NO: 242 - protein, 15 aa, synthetic construct
  REGION 1..15: note = Synthetic: Rv2874
RGKVVLIDFWAYPCI

SEQ ID NO: 243 - protein, 15 aa, synthetic construct
  REGION 1..15: note = Synthetic: Rv1199c
RSRPRRTTRRMDRRT

SEQ ID NO: 244 - protein, 15 aa, synthetic construct
  REGION 1..15: note = Synthetic: Rv1195
SSYAATEVANAAAAS

SEQ ID NO: 245 - protein, 15 aa, synthetic construct
  REGION 1..15: note = Synthetic: Rv1195
SSYAATEVANAAAGQ

SEQ ID NO: 246 - protein, 15 aa, synthetic construct
  REGION 1..15: note = Synthetic: Rv0294
STIFPFRRLFMVAEV

SEQ ID NO: 247 - protein, 15 aa, synthetic construct
  REGION 1..15: note = Synthetic: Rv3136
VTANRAELKALIASN

SEQ ID NO: 248 - protein, 15 aa, synthetic construct
  REGION 1..15: note = Synthetic: Rv1705c
WFINWYLPISQLFYN

SEQ ID NO: 249 - protein, 15 aa, synthetic construct
  REGION 1..15: note = Synthetic: Rv0453
YAAALVAMPTLAELA

SEQ ID NO: 250 - protein, 15 aa, synthetic construct
  REGION 1..15: note = Synthetic: Rv2853
YASVEAANASPLQVA

SEQ ID NO: 251 - protein, 15 aa, synthetic construct
  REGION 1..15: note = Synthetic: Rv2094c
TPVQSQRVDPSAASG

SEQ ID NO: 252 - protein, 15 aa, synthetic construct
  REGION 1..15: note = Synthetic: Rv0440
MAKTIAYDEEARRGL

SEQ ID NO: 253 - protein, 15 aa, synthetic construct
  REGION 1..15: note = Synthetic: Rv3620c
NQAFRNIVNMLHGVR

SEQ ID NO: 254 - protein, 15 aa, synthetic construct
  REGION 1..15: note = Synthetic: Rv3615c
VDLAKSLRIAAKIYS

SEQ ID NO: 255 - protein, 15 aa, synthetic construct
  REGION 1..15: note = Synthetic: Rv3615c
LRIAAKIYSEADEAW

SEQ ID NO: 256 - protein, 15 aa, synthetic construct
  REGION 1..15: note = Synthetic: Rv3020c
AAFQGAHARFVAAAA

SEQ ID NO: 257 - protein, 15 aa, synthetic construct
  REGION 1..15: note = Synthetic: Rv3018c
AVLVATNFFGINTIP

SEQ ID NO: 258 - protein, 15 aa, synthetic construct
  REGION 1..15: note = Synthetic: Rv2031c
AYGSFVRTVSLPVGA

SEQ ID NO: 259 - protein, 15 aa, synthetic construct
  REGION 1..15: note = Synthetic: Rv3019c
MSQIMYNYPAMMAHA

SEQ ID NO: 260 - protein, 15 aa, synthetic construct
  REGION 1..15: note = Synthetic: Rv0287
AAFQAAHARFVAAAA

SEQ ID NO: 261 - protein, 15 aa, synthetic construct
  REGION 1..15: note = Synthetic: Rv3020c
AAGTYVAADAAAASS

SEQ ID NO: 262 - protein, 15 aa, synthetic construct
  REGION 1..15: note = Synthetic: Rv0288
LQSLGAEIAVEQAAL

SEQ ID NO: 263 - protein, 15 aa, synthetic construct
  REGION 1..15: note = Synthetic: Rv1198
AEHQAIIRDVLTASD

SEQ ID NO: 264 - protein, 15 aa, synthetic construct
  REGION 1..15: note = Synthetic: Rv3019c
MSQIMYNYPAMRAHA

SEQ ID NO: 265 - protein, 15 aa, synthetic construct
  REGION 1..15: note = Synthetic: Rv0290
IPVMAYLVGLFAWVL

SEQ ID NO: 266 - protein, 15 aa, synthetic construct
  REGION 1..15: note = Synthetic: Rv0287
AAGTYVAADAAAAST

SEQ ID NO: 267 - protein, 15 aa, synthetic construct
  REGION 1..15: note = Synthetic: Rv1387
GINTIPIAINEAEYV

SEQ ID NO: 268 - protein, 15 aa, synthetic construct
  REGION 1..15: note = Synthetic: Rv0280
GVLVATNFFGINTIP

SEQ ID NO: 269 - protein, 15 aa, synthetic construct
  REGION 1..15: note = Synthetic: Rv1195
VHVSFVMAYPEMLAA

SEQ ID NO: 270 - protein, 15 aa, synthetic construct
  REGION 1..15: note = Synthetic: Rv3135
AAASWDALAAELASA

SEQ ID NO: 271 - protein, 15 aa, synthetic construct
  REGION 1..15: note = Synthetic: Rv3136
AAGGWDSLAAELATT

SEQ ID NO: 272 - protein, 15 aa, synthetic construct
  REGION 1..15: note = Synthetic: Rv0288
EDLVRAYHAMSSTHE

SEQ ID NO: 273 - protein, 15 aa, synthetic construct
  REGION 1..15: note = Synthetic: Rv0288
EDLVRAYHSMSSTHE

SEQ ID NO: 274 - protein, 15 aa, synthetic construct
  REGION 1..15: note = Synthetic: Rv3136
FFGQNTAAIAATEAQ

SEQ ID NO: 275 - protein, 15 aa, synthetic construct
  REGION 1..15: note = Synthetic: Rv3136
LLGQNTAAIAAIEAQ

SEQ ID NO: 276 - protein, 15 aa, synthetic construct
  REGION 1..15: note = Synthetic: Rv2875
AAFSKLPASTIDELK

SEQ ID NO: 277 - protein, 15 aa, synthetic construct
  REGION 1..15: note = Synthetic: Rv0290
GWYLVAATAAAATLR

SEQ ID NO: 278 - protein, 15 aa, synthetic construct
  REGION 1..15: note = Synthetic: Rv2024c
KQQVIAELYEKFFRI

SEQ ID NO: 279 - protein, 15 aa, synthetic construct
  REGION 1..15: note = Synthetic: Rv0290
QLSALWARFPLPVIP

SEQ ID NO: 280 - protein, 15 aa, synthetic construct
  REGION 1..15: note = Synthetic: Rv0288
AMMARDTAEAAKWGG

SEQ ID NO: 281 - protein, 15 aa, synthetic construct
  REGION 1..15: note = Synthetic: Rv3874
KQELDEISTNIRQAG

SEQ ID NO: 282 - protein, 15 aa, synthetic construct
  REGION 1..15: note = Synthetic: Rv0288
MAMMARDTAEAAKWG

SEQ ID NO: 283 - protein, 15 aa, synthetic construct
  REGION 1..15: note = Synthetic: Rv3875
ISEAGQAMASTEGNV

SEQ ID NO: 284 - protein, 15 aa, synthetic construct
  REGION 1..15: note = Synthetic: Rv3875
QKWDATATELNNALQ

SEQ ID NO: 285 - protein, 15 aa, synthetic construct
  REGION 1..15: note = Synthetic: Rv3875
YQGVQQKWDATATEL

SEQ ID NO: 286 - protein, 15 aa, synthetic construct
  REGION 1..15: note = Synthetic: Rv0288
AQWNQAMEDLVRAYH

SEQ ID NO: 287 - protein, 15 aa, synthetic construct
  REGION 1..15: note = Synthetic: Rv3874
EISTNIRQAGVQYSR

SEQ ID NO: 288 - protein, 15 aa, synthetic construct
  REGION 1..15: note = Synthetic: Rv3874
IRQAGVQYSRADEEQ

SEQ ID NO: 289 - protein, 15 aa, synthetic construct
  REGION 1..15: note = Synthetic: Rv3875
NLARTISEAGQAMAS

SEQ ID NO: 290 - protein, 15 aa, synthetic construct
  REGION 1..15: note = Synthetic: Rv3874
AAGTAAQAAVVRFQE

SEQ ID NO: 291 - protein, 15 aa, synthetic construct
  REGION 1..15: note = Synthetic: Rv0288
HEANTMAMMARDTAE

SEQ ID NO: 292 - protein, 15 aa, synthetic construct
  REGION 1..15: note = Synthetic: Rv0288
MSQIMYNYPAMLGHA

SEQ ID NO: 293 - protein, 15 aa, synthetic construct
  REGION 1..15: note = Synthetic: Rv3875
MTEQQWNFAGIEAAA

SEQ ID NO: 294 - protein, 15 aa, synthetic construct
  REGION 1..15: note = Synthetic: Rv0288
SAWQGDTGITYQAWQ

SEQ ID NO: 295 - protein, 15 aa, synthetic construct
  REGION 1..15: note = Synthetic: Rv0288
AMEDLVRAYHAMSST

SEQ ID NO: 296 - protein, 15 aa, synthetic construct
  REGION 1..15: note = Synthetic: Rv0288
DTGITYQAWQAQWNQ

SEQ ID NO: 297 - protein, 15 aa, synthetic construct
  REGION 1..15: note = Synthetic: Rv0288
GDMAGYAGTLQSLGA

SEQ ID NO: 298 - protein, 15 aa, synthetic construct
  REGION 1..15: note = Synthetic: Rv1196
GSASLVAAAQMWDSV

SEQ ID NO: 299 - protein, 15 aa, synthetic construct
  REGION 1..15: note = Synthetic: Rv1196
INSARMYAGPGSASL

SEQ ID NO: 300 - protein, 15 aa, synthetic construct
  REGION 1..15: note = Synthetic: Rv1196
LIATNLLGQNTPAIA

SEQ ID NO: 301 - protein, 15 aa, synthetic construct
  REGION 1..15: note = Synthetic: Rv1196
LLPFEEAPEMTSAGG

SEQ ID NO: 302 - protein, 15 aa, synthetic construct
  REGION 1..15: note = Synthetic: Rv3874
MAEMKTDAATLAQEA

SEQ ID NO: 303 - protein, 15 aa, synthetic construct
  REGION 1..15: note = Synthetic: Rv3804c
MGRDIKVQFQSGGAN

SEQ ID NO: 304 - protein, 15 aa, synthetic construct
  REGION 1..15: note = Synthetic: Rv3875
NNALQNLARTISEAG

SEQ ID NO: 305 - protein, 15 aa, synthetic construct
  REGION 1..15: note = Synthetic: Rv3875
NVTSIHSLLDEGKQS

SEQ ID NO: 306 - protein, 15 aa, synthetic construct
  REGION 1..15: note = Synthetic: Rv1886c
PSMGRDIKVQFQSGG

SEQ ID NO: 307 - protein, 15 aa, synthetic construct
  REGION 1..15: note = Synthetic: Rv0288
QAALQSAWQGDTGIT

SEQ ID NO: 308 - protein, 15 aa, synthetic construct
  REGION 1..15: note = Synthetic: Rv3874
QVESTAGSLQGQWRG

SEQ ID NO: 309 - protein, 15 aa, synthetic construct
  REGION 1..15: note = Synthetic: Rv3875
SAIQGNVTSIHSLLD

SEQ ID NO: 310 - protein, 15 aa, synthetic construct
  REGION 1..15: note = Synthetic: Rv3875
SGSEAYQGVQQKWDA

SEQ ID NO: 311 - protein, 15 aa, synthetic construct
  REGION 1..15: note = Synthetic: Rv1196
TPAIAVNEAEYGEMW

SEQ ID NO: 312 - protein, 15 aa, synthetic construct
  REGION 1..15: note = Synthetic: Rv0288
VRAYHAMSSTHEANT

SEQ ID NO: 313 - protein, 15 aa, synthetic construct
  REGION 1..15: note = Synthetic: Rv0288
YAGTLQSLGAEIAVE

SEQ ID NO: 314 - protein, 15 aa, synthetic construct
  REGION 1..15: note = Synthetic: Rv0288
YNYPAMLGHAGDMAG

SEQ ID NO: 315 - protein, 15 aa, synthetic construct
  REGION 1..15: note = Synthetic: Rv0288
YQAWQAQWNQAMEDL

SEQ ID NO: 316 - protein, 15 aa, synthetic construct
  REGION 1..15: note = Synthetic: Rv1888c
ILLLVAVVALLFTSR

SEQ ID NO: 317 - protein, 15 aa, synthetic construct
  REGION 1..15: note = Synthetic: Rv2095c
EATGLLVALRALADI

SEQ ID NO: 318 - protein, 15 aa, synthetic construct
  REGION 1..15: note = Synthetic: Rv0176
ACLLGLTILLLAVNR

SEQ ID NO: 319 - protein, 15 aa, synthetic construct
  REGION 1..15: note = Synthetic: Rv3874
AEMKTDAATLAQEAG

SEQ ID NO: 320 - protein, 15 aa, synthetic construct
  REGION 1..15: note = Synthetic: Rv3063
AIFMGCYLRFLRPGR

SEQ ID NO: 321 - protein, 15 aa, synthetic construct
  REGION 1..15: note = Synthetic: Rv1351
AQHRHRYLTMVNVGR

SEQ ID NO: 322 - protein, 15 aa, synthetic construct
  REGION 1..15: note = Synthetic: Rv3635
GWLVLIAVLALSLVR

SEQ ID NO: 323 - protein, 15 aa, synthetic construct
  REGION 1..15: note = Synthetic: Rv2051c
ILVGAAAAVVLVAMR

SEQ ID NO: 324 - protein, 15 aa, synthetic construct
  REGION 1..15: note = Synthetic: Rv0176
IRRLTAAVALAAAGA

SEQ ID NO: 325 - protein, 15 aa, synthetic construct
  REGION 1..15: note = Synthetic: Rv1508c
IVVVAVLALGRRIPP

SEQ ID NO: 326 - protein, 15 aa, synthetic construct
  REGION 1..15: note = Synthetic: Rv1349
LIGFALLAFCSVVAR

SEQ ID NO: 327 - protein, 15 aa, synthetic construct
  REGION 1..15: note = Synthetic: Rv1807
AAAAAYEAAFAATVP

SEQ ID NO: 328 - protein, 15 aa, synthetic construct
  REGION 1..15: note = Synthetic: Rv2338c
AAEVLKILLGHGRVY

SEQ ID NO: 329 - protein, 15 aa, synthetic construct
  REGION 1..15: note = Synthetic: Rv1195
AAIHDQFVATLASSA

SEQ ID NO: 330 - protein, 15 aa, synthetic construct
  REGION 1..15: note = Synthetic: Rv3125c
AAIQARAAATAFEAA

SEQ ID NO: 331 - protein, 15 aa, synthetic construct
  REGION 1..15: note = Synthetic: Rv0205
AAVLVLFTLIFFLYG

SEQ ID NO: 332 - protein, 15 aa, synthetic construct
  REGION 1..15: note = Synthetic: Rv3261
ADADIIMLAPSNPVV

SEQ ID NO: 333 - protein, 15 aa, synthetic construct
  REGION 1..15: note = Synthetic: Rv0144
ADAQYPASTAFLATT

SEQ ID NO: 334 - protein, 15 aa, synthetic construct
  REGION 1..15: note = Synthetic: Rv0016c
ADGLRADPRNQRVLL

SEQ ID NO: 335 - protein, 15 aa, synthetic construct
  REGION 1..15: note = Synthetic: Rv1807
AGAQAEGAAAAYEAA

SEQ ID NO: 336 - protein, 15 aa, synthetic construct
  REGION 1..15: note = Synthetic: Rv0590A
AGGIYGDFFNFYLCD

SEQ ID NO: 337 - protein, 15 aa, synthetic construct
  REGION 1..15: note = Synthetic: Rv0284
AGHTHYLIIDDVDQV

SEQ ID NO: 338 - protein, 15 aa, synthetic construct
  REGION 1..15: note = Synthetic: Rv2040c
AILLVLTVLQLRITH

SEQ ID NO: 339 - protein, 15 aa, synthetic construct
  REGION 1..15: note = Synthetic: Rv0676c
ALCLIFIIMLIITRS

SEQ ID NO: 340 - protein, 15 aa, synthetic construct
  REGION 1..15: note = Synthetic: Rv0676c
ALCLIFIIMLITTRS

SEQ ID NO: 341 - protein, 15 aa, synthetic construct
  REGION 1..15: note = Synthetic: Rv1997
ANRWIILGVSAQAIA

SEQ ID NO: 342 - protein, 15 aa, synthetic construct
  REGION 1..15: note = Synthetic: Rv1227c
APVMWALSASLGWIL

SEQ ID NO: 343 - protein, 15 aa, synthetic construct
  REGION 1..15: note = Synthetic: Rv3782
AQEWLRFGWFFLATR

SEQ ID NO: 344 - protein, 15 aa, synthetic construct
  REGION 1..15: note = Synthetic: Rv3782
AQEWLRFGWFFLVTR

SEQ ID NO: 345 - protein, 15 aa, synthetic construct
  REGION 1..15: note = Synthetic: Rv3071
ARFFGRNVNVPLMII

SEQ ID NO: 346 - protein, 15 aa, synthetic construct
  REGION 1..15: note = Synthetic: Rv2024c
ARILLLVPSISLLSQ

SEQ ID NO: 347 - protein, 15 aa, synthetic construct
  REGION 1..15: note = Synthetic: Rv3763
ASGPKVVIDGKDQNV

SEQ ID NO: 348 - protein, 15 aa, synthetic construct
  REGION 1..15: note = Synthetic: Rv3887c
ATRFRPIIRLTVEWL

SEQ ID NO: 349 - protein, 15 aa, synthetic construct
  REGION 1..15: note = Synthetic: Rv3887c
ATRVRPIIRLTVEWL

SEQ ID NO: 350 - protein, 15 aa, synthetic construct
  REGION 1..15: note = Synthetic: Rv1894c
AVGFTPEQLEIELNW

SEQ ID NO: 351 - protein, 15 aa, synthetic construct
  REGION 1..15: note = Synthetic: Rv3436c
AVLRRLEWHFTLVFA

SEQ ID NO: 352 - protein, 15 aa, synthetic construct
  REGION 1..15: note = Synthetic: Rv1886c
DIKVQFQSGGNNSPA

SEQ ID NO: 353 - protein, 15 aa, synthetic construct
  REGION 1..15: note = Synthetic: Rv3432c
DLVWDFRLPRVVSIN

SEQ ID NO: 354 - protein, 15 aa, synthetic construct
  REGION 1..15: note = Synthetic: Rv0107c
DQLVCRVVVPAVATT

SEQ ID NO: 355 - protein, 15 aa, synthetic construct
  REGION 1..15: note = Synthetic: Rv0107c
DQLVRRVVVPAVATT

SEQ ID NO: 356 - protein, 15 aa, synthetic construct
  REGION 1..15: note = Synthetic: Rv3784
DRYINWAKDQPQYPY

SEQ ID NO: 357 - protein, 15 aa, synthetic construct
  REGION 1..15: note = Synthetic: Rv0040c
DSQKLLAWQTTNASM

SEQ ID NO: 358 - protein, 15 aa, synthetic construct
  REGION 1..15: note = Synthetic: Rv2095c
EATGLLVALRALANI

SEQ ID NO: 359 - protein, 15 aa, synthetic construct
  REGION 1..15: note = Synthetic: Rv3646c
EDALRLLSLPRVVGV

SEQ ID NO: 360 - protein, 15 aa, synthetic construct
  REGION 1..15: note = Synthetic: Rv0440
EDAVRNAKAAVEEGI

SEQ ID NO: 361 - protein, 15 aa, synthetic construct
  REGION 1..15: note = Synthetic: Rv0288
EDLVRAYHAMSRTHE

SEQ ID NO: 362 - protein, 15 aa, synthetic construct
  REGION 1..15: note = Synthetic: Rv0361
EDQVRQAIQSLDIAI

SEQ ID NO: 363 - protein, 15 aa, synthetic construct
  REGION 1..15: note = Synthetic: Rv0876c
ELWVGFTAVSALLIL

SEQ ID NO: 364 - protein, 15 aa, synthetic construct
  REGION 1..15: note = Synthetic: Rv0573c
EQDLRYLRGLGQFSD

SEQ ID NO: 365 - protein, 15 aa, synthetic construct
  REGION 1..15: note = Synthetic: Rv2096c
ERLVNLVIALLSTRG

SEQ ID NO: 366 - protein, 15 aa, synthetic construct
  REGION 1..15: note = Synthetic: Rv2032
ESLRLYDSSYHAELF

SEQ ID NO: 367 - protein, 15 aa, synthetic construct
  REGION 1..15: note = Synthetic: Rv0276
ESPVSYHAFDPELQL

SEQ ID NO: 368 - protein, 15 aa, synthetic construct
  REGION 1..15: note = Synthetic: Rv0284
EVFLVIDNLYGFGRD

SEQ ID NO: 369 - protein, 15 aa, synthetic construct
  REGION 1..15: note = Synthetic: Rv3794
FCLFGVLFVLLRRGR

SEQ ID NO: 370 - protein, 15 aa, synthetic construct
  REGION 1..15: note = Synthetic: Rv0284
FFPFVLLLAATALYR

SEQ ID NO: 371 - protein, 15 aa, synthetic construct
  REGION 1..15: note = Synthetic: Rv0731c
FGEVIYVSAELKQKH

SEQ ID NO: 372 - protein, 15 aa, synthetic construct
  REGION 1..15: note = Synthetic: Rv0276
FNLMLWDLDRRMRRG

SEQ ID NO: 373 - protein, 15 aa, synthetic construct
  REGION 1..15: note = Synthetic: Rv1894c
FPIFAVTHCRDVVVA

SEQ ID NO: 374 - protein, 15 aa, synthetic construct
  REGION 1..15: note = Synthetic: Rv1629
FRAFYALPAENFKTR

SEQ ID NO: 375 - protein, 15 aa, synthetic construct
  REGION 1..15: note = Synthetic: Rv1807
GAAAAYEAAFAATVP

SEQ ID NO: 376 - protein, 15 aa, synthetic construct
  REGION 1..15: note = Synthetic: Rv2346c
GAMIRAQAGLLEAEH

SEQ ID NO: 377 - protein, 15 aa, synthetic construct
  REGION 1..15: note = Synthetic: Rv0160c
GGLLARFPGFYIPFL

SEQ ID NO: 378 - protein, 15 aa, synthetic construct
  REGION 1..15: note = Synthetic: Rv3493c
GIDEISDNASVLVSV

SEQ ID NO: 379 - protein, 15 aa, synthetic construct
  REGION 1..15: note = Synthetic: Rv1173
GIGRPARQRTTTYAL

SEQ ID NO: 380 - protein, 15 aa, synthetic construct
  REGION 1..15: note = Synthetic: Rv0284
GILIVGMIVALVATG

SEQ ID NO: 381 - protein, 15 aa, synthetic construct
  REGION 1..15: note = Synthetic: Rv0694
GITVADNVAAFSELG

SEQ ID NO: 382 - protein, 15 aa, synthetic construct
  REGION 1..15: note = Synthetic: Rv3910
GLSSVFMAILNTRNM

SEQ ID NO: 383 - protein, 15 aa, synthetic construct
  REGION 1..15: note = Synthetic: Rv3910
GLSSVFMAILNTRNV

SEQ ID NO: 384 - protein, 15 aa, synthetic construct
  REGION 1..15: note = Synthetic: Rv0017c
GLTAILMLYSIVIIR

SEQ ID NO: 385 - protein, 15 aa, synthetic construct
  REGION 1..15: note = Synthetic: Rv1890c
GNGYRLTGKLHIRGK

SEQ ID NO: 386 - protein, 15 aa, synthetic construct
  REGION 1..15: note = Synthetic: Rv3252c
GPILLYVLLPLLDLR

SEQ ID NO: 387 - protein, 15 aa, synthetic construct
  REGION 1..15: note = Synthetic: Rv3818
GSTADFTGTTLNSLR

SEQ ID NO: 388 - protein, 15 aa, synthetic construct
  REGION 1..15: note = Synthetic: Rv1122
GSWLLDLTAIALRES

SEQ ID NO: 389 - protein, 15 aa, synthetic construct
  REGION 1..15: note = Synthetic: Rv1888c
ILLLVAVVALLFTGR

SEQ ID NO: 390 - protein, 15 aa, synthetic construct
  REGION 1..15: note = Synthetic: Rv3383c
ILLRDALHATAVRIL

SEQ ID NO: 391 - protein, 15 aa, synthetic construct
  REGION 1..15: note = Synthetic: Rv1681
IRLALSLGFRVRVAA

SEQ ID NO: 392 - protein, 15 aa, synthetic construct
  REGION 1..15: note = Synthetic: Rv1565c
IRRLLPALVVVLAGC

SEQ ID NO: 393 - protein, 15 aa, synthetic construct
  REGION 1..15: note = Synthetic: Rv0268c
ISAERYESLMEELED

SEQ ID NO: 394 - protein, 15 aa, synthetic construct
  REGION 1..15: note = Synthetic: Rv0284
ISPQTLFFPFVLLLA

SEQ ID NO: 395 - protein, 15 aa, synthetic construct
  REGION 1..15: note = Synthetic: Rv3796
IVDYYNFFLSGGFLA

SEQ ID NO: 396 - protein, 15 aa, synthetic construct
  REGION 1..15: note = Synthetic: Rv1813c
KAWHQRTPARAEQVA

SEQ ID NO: 397 - protein, 15 aa, synthetic construct
  REGION 1..15: note = Synthetic: Rv2024c
KDDIFYYVYGLLHDP

SEQ ID NO: 398 - protein, 15 aa, synthetic construct
  REGION 1..15: note = Synthetic: Rv1442
KTPEWAAALSGLAAG

SEQ ID NO: 399 - protein, 15 aa, synthetic construct
  REGION 1..15: note = Synthetic: Rv1111c
LGTAAGVLLIGLVRW

SEQ ID NO: 400 - protein, 15 aa, synthetic construct
  REGION 1..15: note = Synthetic: Rv3796
LHRAFAYTSNIFIRD

SEQ ID NO: 401 - protein, 15 aa, synthetic construct
  REGION 1..15: note = Synthetic: Rv0284
LIIDDVDQVPDSPAM

SEQ ID NO: 402 - protein, 15 aa, synthetic construct
  REGION 1..15: note = Synthetic: Rv0174
LILFAIVSVVAIVVL

SEQ ID NO: 403 - protein, 15 aa, synthetic construct
  REGION 1..15: note = Synthetic: Rv0585c
LPFVILIGLIVSRQW

SEQ ID NO: 404 - protein, 15 aa, synthetic construct
  REGION 1..15: note = Synthetic: Rv0290
LPLRRLLGLVAAGLD

SEQ ID NO: 405 - protein, 15 aa, synthetic construct
  REGION 1..15: note = Synthetic: Rv1599
LPTVRPIVAAVAERG

SEQ ID NO: 406 - protein, 15 aa, synthetic construct
  REGION 1..15: note = Synthetic: Rv3365c
LRRNSANLLVLAGAQ

SEQ ID NO: 407 - protein, 15 aa, synthetic construct
  REGION 1..15: note = Synthetic: Rv0267
LSRAGLFFVPLAVAA

SEQ ID NO: 408 - protein, 15 aa, synthetic construct
  REGION 1..15: note = Synthetic: Rv3557c
LVYRFIRDTTWVSVR

SEQ ID NO: 409 - protein, 15 aa, synthetic construct
  REGION 1..15: note = Synthetic: Rv3522
MLMPCFAQLYDELGI

SEQ ID NO: 410 - protein, 15 aa, synthetic construct
  REGION 1..15: note = Synthetic: Rv3782
NRGYVLSQPGLRKLL

SEQ ID NO: 411 - protein, 15 aa, synthetic construct
  REGION 1..15: note = Synthetic: Rv3458c
NVPSYRVSQYDIVDV

SEQ ID NO: 412 - protein, 15 aa, synthetic construct
  REGION 1..15: note = Synthetic: Rv0244c
NVRDQVFNLFEVLGV

SEQ ID NO: 413 - protein, 15 aa, synthetic construct
  REGION 1..15: note = Synthetic: Rv3818
PDWVNTIFLSTRFRA

SEQ ID NO: 414 - protein, 15 aa, synthetic construct
  REGION 1..15: note = Synthetic: Rv0235c
QLYQYRFTTVAELRR

SEQ ID NO: 415 - protein, 15 aa, synthetic construct
  REGION 1..15: note = Synthetic: Rv0290
QSGFIAAAVLLSVLG

SEQ ID NO: 416 - protein, 15 aa, synthetic construct
  REGION 1..15: note = Synthetic: Rv0836c
RATFRALGSTGHRFL

SEQ ID NO: 417 - protein, 15 aa, synthetic construct
  REGION 1..15: note = Synthetic: Rv3182
RILFAFDPARQAILL

SEQ ID NO: 418 - protein, 15 aa, synthetic construct
  REGION 1..15: note = Synthetic: Rv3125c
SMLFLPVRILTSPIT

SEQ ID NO: 419 - protein, 15 aa, synthetic construct
  REGION 1..15: note = Synthetic: Rv2095c
SPPGLPVAAVAEQAP

SEQ ID NO: 420 - protein, 15 aa, synthetic construct
  REGION 1..15: note = Synthetic: Rv1196
SSAGLMVAAASPYVA

SEQ ID NO: 421 - protein, 15 aa, synthetic construct
  REGION 1..15: note = Synthetic: Rv3635
SSWRPVLLIPLTFAL

SEQ ID NO: 422 - protein, 15 aa, synthetic construct
  REGION 1..15: note = Synthetic: Rv1196
TATATLLPFEEAPEM

SEQ ID NO: 423 - protein, 15 aa, synthetic construct
  REGION 1..15: note = Synthetic: Rv1005c
TTPSVLFLLLKTIAT

SEQ ID NO: 424 - protein, 15 aa, synthetic construct
  REGION 1..15: note = Synthetic: Rv3645
TVIILFLAGAVVNLK

SEQ ID NO: 425 - protein, 15 aa, synthetic construct
  REGION 1..15: note = Synthetic: Rv3148
VLMMELNRISSHLVA

SEQ ID NO: 426 - protein, 15 aa, synthetic construct
  REGION 1..15: note = Synthetic: Rv1565c
WPLLIFWLSYTGHRH

SEQ ID NO: 427 - protein, 15 aa, synthetic construct
  REGION 1..15: note = Synthetic: Rv0266c
YLRIAVANMANAVKK

SEQ ID NO: 428 - protein, 15 aa, synthetic construct
  REGION 1..15: note = Synthetic: Rv0276
YLYPIVALRIRGIAL

SEQ ID NO: 429 - protein, 15 aa, synthetic construct
  REGION 1..15: note = Synthetic: Rv0288
AMMARDTAEAAKWGG

SEQ ID NO: 430 - protein, 15 aa, synthetic construct
  REGION 1..15: note = Synthetic: Rv3874
KQELDEISTNIRQAG

SEQ ID NO: 431 - protein, 15 aa, synthetic construct
  REGION 1..15: note = Synthetic: Rv0288
MAMMARDTAEAAKWG

SEQ ID NO: 432 - protein, 15 aa, synthetic construct
  REGION 1..15: note = Synthetic: Rv3615c
VDLAKSLRIAAKIYS

SEQ ID NO: 433 - protein, 15 aa, synthetic construct
  REGION 1..15: note = Synthetic: Rv3875
ISEAGQAMASTEGNV

SEQ ID NO: 434 - protein, 15 aa, synthetic construct
  REGION 1..15: note = Synthetic: Rv3615c
LRIAAKIYSEADEAW

SEQ ID NO: 435 - protein, 15 aa, synthetic construct
  REGION 1..15: note = Synthetic: Rv3875
QKWDATATELNNALQ

SEQ ID NO: 436 - protein, 15 aa, synthetic construct
  REGION 1..15: note = Synthetic: Rv3875
YQGVQQKWDATATEL

SEQ ID NO: 437 - protein, 15 aa, synthetic construct
  REGION 1..15: note = Synthetic: Rv3020c
AAFQGAHARFVAAAA

SEQ ID NO: 438 - protein, 15 aa, synthetic construct
  REGION 1..15: note = Synthetic: Rv0288
AQWNQAMEDLVRAYH

SEQ ID NO: 439 - protein, 15 aa, synthetic construct
  REGION 1..15: note = Synthetic: Rv3018c
AVLVATNFFGINTIP

SEQ ID NO: 440 - protein, 15 aa, synthetic construct
  REGION 1..15: note = Synthetic: Rv2031c
AYGSFVRTVSLPVGA

SEQ ID NO: 441 - protein, 15 aa, synthetic construct
  REGION 1..15: note = Synthetic: Rv3874
EISTNIRQAGVQYSR

SEQ ID NO: 442 - protein, 15 aa, synthetic construct
  REGION 1..15: note = Synthetic: Rv3874
IRQAGVQYSRADEEQ

SEQ ID NO: 443 - protein, 15 aa, synthetic construct
  REGION 1..15: note = Synthetic: Rv3875
NLARTISEAGQAMAS

SEQ ID NO: 444 - protein, 15 aa, synthetic construct
  REGION 1..15: note = Synthetic: Rv0287
AAFQAAHARFVAAAA

SEQ ID NO: 445 - protein, 15 aa, synthetic construct
  REGION 1..15: note = Synthetic: Rv3874
AAGTAAQAAVVRFQE

SEQ ID NO: 446 - protein, 15 aa, synthetic construct
  REGION 1..15: note = Synthetic: Rv3020c
AAGTYVAADAAAASS

SEQ ID NO: 447 - protein, 15 aa, synthetic construct
  REGION 1..15: note = Synthetic: Rv1198
AEHQAIIRDVLTASD

SEQ ID NO: 448 - protein, 15 aa, synthetic construct
  REGION 1..15: note = Synthetic: Rv0288
HEANTMAMMARDTAE

SEQ ID NO: 449 - protein, 15 aa, synthetic construct
  REGION 1..15: note = Synthetic: Rv0288
LQSLGAEIAVEQAAL

SEQ ID NO: 450 - protein, 15 aa, synthetic construct
  REGION 1..15: note = Synthetic: Rv0288
MSQIMYNYPAMLGHA

SEQ ID NO: 451 - protein, 15 aa, synthetic construct
  REGION 1..15: note = Synthetic: Rv3019c
MSQIMYNYPAMMAHA

SEQ ID NO: 452 - protein, 15 aa, synthetic construct
  REGION 1..15: note = Synthetic: Rv3875
MTEQQWNFAGIEAAA

SEQ ID NO: 453 - protein, 15 aa, synthetic construct
  REGION 1..15: note = Synthetic: Rv0288
SAWQGDTGITYQAWQ

SEQ ID NO: 454 - protein, 15 aa, synthetic construct
  REGION 1..15: note = Synthetic: Rv3135
AAASWDALAAELASA

SEQ ID NO: 455 - protein, 15 aa, synthetic construct
  REGION 1..15: note = Synthetic: Rv2875
AAFSKLPASTIDELK

SEQ ID NO: 456 - protein, 15 aa, synthetic construct
  REGION 1..15: note = Synthetic: Rv3136
AAGGWDSLAAELATT

SEQ ID NO: 457 - protein, 15 aa, synthetic construct
  REGION 1..15: note = Synthetic: Rv0287
AAGTYVAADAAAAST

SEQ ID NO: 458 - protein, 15 aa, synthetic construct
  REGION 1..15: note = Synthetic: Rv0288
AMEDLVRAYHAMSST

SEQ ID NO: 459 - protein, 15 aa, synthetic construct
  REGION 1..15: note = Synthetic: Rv0288
DTGITYQAWQAQWNQ

SEQ ID NO: 460 - protein, 15 aa, synthetic construct
  REGION 1..15: note = Synthetic: Rv0288
EDLVRAYHAMSSTHE

SEQ ID NO: 461 - protein, 15 aa, synthetic construct
  REGION 1..15: note = Synthetic: Rv0288
EDLVRAYHSMSSTHE

SEQ ID NO: 462 - protein, 15 aa, synthetic construct
  REGION 1..15: note = Synthetic: Rv3136
FFGQNTAAIAATEAQ

SEQ ID NO: 463 - protein, 15 aa, synthetic construct
  REGION 1..15: note = Synthetic: Rv0288
GDMAGYAGTLQSLGA

SEQ ID NO: 464 - protein, 15 aa, synthetic construct
  REGION 1..15: note = Synthetic: Rv1387
GINTIPIAINEAEYV

SEQ ID NO: 465 - protein, 15 aa, synthetic construct
  REGION 1..15: note = Synthetic: Rv1196
GSASLVAAAQMWDSV

SEQ ID NO: 466 - protein, 15 aa, synthetic construct
  REGION 1..15: note = Synthetic: Rv0280
GVLVATNFFGINTIP

SEQ ID NO: 467 - protein, 15 aa, synthetic construct
  REGION 1..15: note = Synthetic: Rv0290
GWYLVAATAAAATLR

SEQ ID NO: 468 - protein, 15 aa, synthetic construct
  REGION 1..15: note = Synthetic: Rv1196
INSARMYAGPGSASL

SEQ ID NO: 469 - protein, 15 aa, synthetic construct
  REGION 1..15: note = Synthetic: Rv0290
IPVMAYLVGLFAWVL

SEQ ID NO: 470 - protein, 15 aa, synthetic construct
  REGION 1..15: note = Synthetic: Rv2024c
KQQVIAELYEKFFRI

SEQ ID NO: 471 - protein, 15 aa, synthetic construct
  REGION 1..15: note = Synthetic: Rv1196
LIATNLLGQNTPAIA

SEQ ID NO: 472 - protein, 15 aa, synthetic construct
  REGION 1..15: note = Synthetic: Rv3136
LLGQNTAAIAAIEAQ

SEQ ID NO: 473 - protein, 15 aa, synthetic construct
  REGION 1..15: note = Synthetic: Rv1196
LLPFEEAPEMTSAGG

SEQ ID NO: 474 - protein, 15 aa, synthetic construct
  REGION 1..15: note = Synthetic: Rv3874
MAEMKTDAATLAQEA

SEQ ID NO: 475 - protein, 15 aa, synthetic construct
  REGION 1..15: note = Synthetic: Rv3804c
MGRDIKVQFQSGGAN

SEQ ID NO: 476 - protein, 15 aa, synthetic construct
  REGION 1..15: note = Synthetic: Rv3019c
MSQIMYNYPAMRAHA

SEQ ID NO: 477 - protein, 15 aa, synthetic construct
  REGION 1..15: note = Synthetic: Rv3875
NNALQNLARTISEAG

SEQ ID NO: 478 - protein, 15 aa, synthetic construct
  REGION 1..15: note = Synthetic: Rv3875
NVTSIHSLLDEGKQS

SEQ ID NO: 479 - protein, 15 aa, synthetic construct
  REGION 1..15: note = Synthetic: Rv1886c
PSMGRDIKVQFQSGG

SEQ ID NO: 480 - protein, 15 aa, synthetic construct
  REGION 1..15: note = Synthetic: Rv0288
QAALQSAWQGDTGIT

SEQ ID NO: 481 - protein, 15 aa, synthetic construct
  REGION 1..15: note = Synthetic: Rv0290
QLSALWARFPLPVIP

SEQ ID NO: 482 - protein, 15 aa, synthetic construct
  REGION 1..15: note = Synthetic: Rv3874
QVESTAGSLQGQWRG

SEQ ID NO: 483 - protein, 15 aa, synthetic construct
  REGION 1..15: note = Synthetic: Rv3875
SAIQGNVTSIHSLLD

SEQ ID NO: 484 - protein, 15 aa, synthetic construct
  REGION 1..15: note = Synthetic: Rv3875
SGSEAYQGVQQKWDA

SEQ ID NO: 485 - protein, 15 aa, synthetic construct
  REGION 1..15: note = Synthetic: Rv1196
TPAIAVNEAEYGEMW

SEQ ID NO: 486 - protein, 15 aa, synthetic construct
  REGION 1..15: note = Synthetic: Rv1195
VHVSFVMAYPEMLAA

SEQ ID NO: 487 - protein, 15 aa, synthetic construct
  REGION 1..15: note = Synthetic: Rv0288
VRAYHAMSSTHEANT

SEQ ID NO: 488 - protein, 15 aa, synthetic construct
  REGION 1..15: note = Synthetic: Rv0288
YAGTLQSLGAEIAVE

SEQ ID NO: 489 - protein, 15 aa, synthetic construct
  REGION 1..15: note = Synthetic: Rv0288
YNYPAMLGHAGDMAG

SEQ ID NO: 490 - protein, 15 aa, synthetic construct
  REGION 1..15: note = Synthetic: Rv0288
YQAWQAQWNQAMEDL

SEQ ID NO: 491 - protein, 15 aa, synthetic construct
  REGION 1..15: note = Synthetic: Rv1802
AAAQASAAAAAYEAA

SEQ ID NO: 492 - protein, 15 aa, synthetic construct
  REGION 1..15: note = Synthetic: Rv1196
AAERGPGQMLGGLPV

SEQ ID NO: 493 - protein, 15 aa, synthetic construct
  REGION 1..15: note = Synthetic: Rv2823c
AAFSRMLSLFFRQHI

SEQ ID NO: 494 - protein, 15 aa, synthetic construct
  REGION 1..15: note = Synthetic: Rv0290
AAGAQLLWQLPLLSI

SEQ ID NO: 495 - protein, 15 aa, synthetic construct
  REGION 1..15: note = Synthetic: Rv0290
AAGVAAWSLIALMIP

SEQ ID NO: 496 - protein, 15 aa, synthetic construct
  REGION 1..15: note = Synthetic: Rv1788
AAIHEMFVNTLQMSS

SEQ ID NO: 497 - protein, 15 aa, synthetic construct
  REGION 1..15: note = Synthetic: Rv1791
AAIHEMFVNTLVASS

SEQ ID NO: 498 - protein, 15 aa, synthetic construct
  REGION 1..15: note = Synthetic: Rv3021c
AALPAVGAAAGAPAA

SEQ ID NO: 499 - protein, 15 aa, synthetic construct
  REGION 1..15: note = Synthetic: Rv2874
AALPLLFFALAGQRI

SEQ ID NO: 500 - protein, 15 aa, synthetic construct
  REGION 1..15: note = Synthetic: Rv1196
AANQLMNNVPQALQQ

SEQ ID NO: 501 - protein, 15 aa, synthetic construct
  REGION 1..15: note = Synthetic: Rv0291
AARLLSIRAMSTKFS

SEQ ID NO: 502 - protein, 15 aa, synthetic construct
  REGION 1..15: note = Synthetic: Rv3015c
AASLLDEDMDALEEA

SEQ ID NO: 503 - protein, 15 aa, synthetic construct
  REGION 1..15: note = Synthetic: Rv1789
AATQARAAAAAFEAA

SEQ ID NO: 504 - protein, 15 aa, synthetic construct
  REGION 1..15: note = Synthetic: Rv0291
AAVDKDAVIVAAAGN

SEQ ID NO: 505 - protein, 15 aa, synthetic construct
  REGION 1..15: note = Synthetic: Rv1196
AAVEEASDTAAANQL

SEQ ID NO: 506 - protein, 15 aa, synthetic construct
  REGION 1..15: note = Synthetic: Rv3021c
AAVPAVGAAAGAPAA

SEQ ID NO: 507 - protein, 15 aa, synthetic construct
  REGION 1..15: note = Synthetic: Rv3874
AAVVRFQEAANKQKQ

SEQ ID NO: 508 - protein, 15 aa, synthetic construct
  REGION 1..15: note = Synthetic: Rv1196
AAYETAYGLTVPPPV

SEQ ID NO: 509 - protein, 15 aa, synthetic construct
  REGION 1..15: note = Synthetic: Rv3874
ADEEQQQALSSQMGF

SEQ ID NO: 510 - protein, 15 aa, synthetic construct
  REGION 1..15: note = Synthetic: Rv2123
ADYLRMWIQAATVMS

SEQ ID NO: 511 - protein, 15 aa, synthetic construct
  REGION 1..15: note = Synthetic: Rv0256c
AEAPAAAAAPEEQVQ

SEQ ID NO: 512 - protein, 15 aa, synthetic construct
  REGION 1..15: note = Synthetic: Rv3619c
AEHQAIISDVLTASD

SEQ ID NO: 513 - protein, 15 aa, synthetic construct
  REGION 1..15: note = Synthetic: Rv1198
AEHQAIVRDVLAAGD

SEQ ID NO: 514 - protein, 15 aa, synthetic construct
  REGION 1..15: note = Synthetic: Rv1198
AEHQAIVRDVLAASD

SEQ ID NO: 515 - protein, 15 aa, synthetic construct
  REGION 1..15: note = Synthetic: Rv3024c
AEKFKEDVINDFVSS

SEQ ID NO: 516 - protein, 15 aa, synthetic construct
  REGION 1..15: note = Synthetic: Rv1361c
AELMILIATNLLGQN

SEQ ID NO: 517 - protein, 15 aa, synthetic construct
  REGION 1..15: note = Synthetic: Rv3804c
AGCQTYKWETFLTSE

SEQ ID NO: 518 - protein, 15 aa, synthetic construct
  REGION 1..15: note = Synthetic: Rv3620c
AGRFEVHAQTVEDEA

SEQ ID NO: 519 - protein, 15 aa, synthetic construct
  REGION 1..15: note = Synthetic: Rv3619
AGSLEAEHQAIISDV

SEQ ID NO: 520 - protein, 15 aa, synthetic construct
  REGION 1..15: note = Synthetic: Rv3874
AGSLQGQWRGAAGTA

SEQ ID NO: 521 - protein, 15 aa, synthetic construct
  REGION 1..15: note = Synthetic: Rv1886c
AGSLSALLDPSQGMG

SEQ ID NO: 522 - protein, 15 aa, synthetic construct
  REGION 1..15: note = Synthetic: Rv3115
AGWLAFFRDLVARGL

SEQ ID NO: 523 - protein, 15 aa, synthetic construct
  REGION 1..15: note = Synthetic: Rv3024c
AHGETVSAVAELIGD

SEQ ID NO: 524 - protein, 15 aa, synthetic construct
  REGION 1..15: note = Synthetic: Rv0292
AILRRRRRIAEPATC

SEQ ID NO: 525 - protein, 15 aa, synthetic construct
  REGION 1..15: note = Synthetic: Rv0294
AKLMRDIPFRVGAVV

SEQ ID NO: 526 - protein, 15 aa, synthetic construct
  REGION 1..15: note = Synthetic: Rv3804c
ALGATPNTGPAPQGA

SEQ ID NO: 527 - protein, 15 aa, synthetic construct
  REGION 1..15: note = Synthetic: Rv1172c
ALLPRAGAAAAAALP

SEQ ID NO: 528 - protein, 15 aa, synthetic construct
  REGION 1..15: note = Synthetic: Rv1196
ALPPEINSARMYAGP

SEQ ID NO: 529 - protein, 15 aa, synthetic construct
  REGION 1..15: note = Synthetic: Rv3025c
ALQSHDDVALVSVMW

SEQ ID NO: 530 - protein, 15 aa, synthetic construct
  REGION 1..15: note = Synthetic: Rv3022c
ALSAEYAAVAQELSV

SEQ ID NO: 531 - protein, 15 aa, synthetic construct
  REGION 1..15: note = Synthetic: Rv1172c
ALSRVHSMFLGTGGS

SEQ ID NO: 532 - protein, 15 aa, synthetic construct
  REGION 1..15: note = Synthetic: Rv1172c
ALSRVQSMFLGTGGS

SEQ ID NO: 533 - protein, 15 aa, synthetic construct
  REGION 1..15: note = Synthetic: Rv0291
ALSVLVGLTAATVAI

SEQ ID NO: 534 - protein, 15 aa, synthetic construct
  REGION 1..15: note = Synthetic: Rv0292
ALTALIRDPPADSTG

SEQ ID NO: 535 - protein, 15 aa, synthetic construct
  REGION 1..15: note = Synthetic: Rv3620c
AMRDMAGRFEVHAQT

SEQ ID NO: 536 - protein, 15 aa, synthetic construct
  REGION 1..15: note = Synthetic: Rv2875
ANATVYMIDSVLMPP

SEQ ID NO: 537 - protein, 15 aa, synthetic construct
  REGION 1..15: note = Synthetic: Rv1172c
APQINFFYYLGEPIV

SEQ ID NO: 538 - protein, 15 aa, synthetic construct
  REGION 1..15: note = Synthetic: Rv1705c
APYVAWMRATAIQAE

SEQ ID NO: 539 - protein, 15 aa, synthetic construct
  REGION 1..15: note = Synthetic: Rv3874
AQAAVVRFQEAANKQ

SEQ ID NO: 540 - protein, 15 aa, synthetic construct
  REGION 1..15: note = Synthetic: Rv0293c
AQAVYDFRSIVDYLR

SEQ ID NO: 541 - protein, 15 aa, synthetic construct
  REGION 1..15: note = Synthetic: Rv1788
AQIYQAVSAQAAAIH

SEQ ID NO: 542 - protein, 15 aa, synthetic construct
  REGION 1..15: note = Synthetic: Rv1317c
AQLGYTIRQLERLLQ

SEQ ID NO: 543 - protein, 15 aa, synthetic construct
  REGION 1..15: note = Synthetic: Rv1808
AQLSQLISLLPSTLQ

SEQ ID NO: 544 - protein, 15 aa, synthetic construct
  REGION 1..15: note = Synthetic: Rv1196
AQNGVQAMSSLGSSL

SEQ ID NO: 545 - protein, 15 aa, synthetic construct
  REGION 1..15: note = Synthetic: Rv2024c
ARILRQLATPISVII

SEQ ID NO: 546 - protein, 15 aa, synthetic construct
  REGION 1..15: note = Synthetic: Rv0256c
ARMWIQAATTMASYQ

SEQ ID NO: 547 - protein, 15 aa, synthetic construct
  REGION 1..15: note = Synthetic: Rv3115
ARTDLLAFTAFPKQI

SEQ ID NO: 548 - protein, 15 aa, synthetic construct
  REGION 1..15: note = Synthetic: Rv3115
ASIIRLVGAVLAEQH

SEQ ID NO: 549 - protein, 15 aa, synthetic construct
  REGION 1..15: note = Synthetic: Rv0291
ATEVVRRLTATAHRG

SEQ ID NO: 550 - protein, 15 aa, synthetic construct
  REGION 1..15: note = Synthetic: Rv3620c
ATSLDTMTQMNQAFR

SEQ ID NO: 551 - protein, 15 aa, synthetic construct
  REGION 1..15: note = Synthetic: Rv3012c
AVDGRFAVPQILGDE

SEQ ID NO: 552 - protein, 15 aa, synthetic construct
  REGION 1..15: note = Synthetic: Rv1366
AVHVWLRLPAGRVEI

SEQ ID NO: 553 - protein, 15 aa, synthetic construct
  REGION 1..15: note = Synthetic: Rv0690c
AVPLRLLGGLHRMVL

SEQ ID NO: 554 - protein, 15 aa, synthetic construct
  REGION 1..15: note = Synthetic: Rv2608
AWHGPASLAMTRAAS

SEQ ID NO: 555 - protein, 15 aa, synthetic construct
  REGION 1..15: note = Synthetic: Rv1789
AWMSAAAAQAEQAAT

SEQ ID NO: 556 - protein, 15 aa, synthetic construct
  REGION 1..15: note = Synthetic: Rv0298
AYAQRVYQANRAAGS

SEQ ID NO: 557 - protein, 15 aa, synthetic construct
  REGION 1..15: note = Synthetic: Rv0987
CILAWILVRIINVRS

SEQ ID NO: 558 - protein, 15 aa, synthetic construct
  REGION 1..15: note = Synthetic: Rv3619
DAHGAMIRAQAGSLE

SEQ ID NO: 559 - protein, 15 aa, synthetic construct
  REGION 1..15: note = Synthetic: Rv2608
DALSGLGLPPPWQPA

SEQ ID NO: 560 - protein, 15 aa, synthetic construct
  REGION 1..15: note = Synthetic: Rv0294
DESWQQFRQELIPLL

SEQ ID NO: 561 - protein, 15 aa, synthetic construct
  REGION 1..15: note = Synthetic: Rv0129c
DPMVQIPRLVANNTR

SEQ ID NO: 562 - protein, 15 aa, synthetic construct
  REGION 1..15: note = Synthetic: Rv0289
DRWLDLRYVGPASAD

SEQ ID NO: 563 - protein, 15 aa, synthetic construct
  REGION 1..15: note = Synthetic: Rv2608
DVDWAEVAADLQQGA

SEQ ID NO: 564 - protein, 15 aa, synthetic construct
  REGION 1..15: note = Synthetic: Rv3018c
DYVRMWVQAATAMSA

SEQ ID NO: 565 - protein, 15 aa, synthetic construct
  REGION 1..15: note = Synthetic: Rv3018c
DYVRMWVQAATVMSA

SEQ ID NO: 566 - protein, 15 aa, synthetic construct
  REGION 1..15: note = Synthetic: Rv3875
EGKQSLTKLAAAWGG

SEQ ID NO: 567 - protein, 15 aa, synthetic construct
  REGION 1..15: note = Synthetic: Rv0299
EHELYVAVLSNALHR

SEQ ID NO: 568 - protein, 15 aa, synthetic construct
  REGION 1..15: note = Synthetic: Rv0292
EIGWEAGTAAPDEIP

SEQ ID NO: 569 - protein, 15 aa, synthetic construct
  REGION 1..15: note = Synthetic: Rv3018c
EIVQFLEETFAAYDQ

SEQ ID NO: 570 - protein, 15 aa, synthetic construct
  REGION 1..15: note = Synthetic: Rv3022c
ELFVAAYVPYVAWLV

SEQ ID NO: 571 - protein, 15 aa, synthetic construct
  REGION 1..15: note = Synthetic: Rv3619
EQANAHGQKVQAAGN

SEQ ID NO: 572 - protein, 15 aa, synthetic construct
  REGION 1..15: note = Synthetic: Rv3875
EQQWNFAGIEAAASA

SEQ ID NO: 573 - protein, 15 aa, synthetic construct
  REGION 1..15: note = Synthetic: Rv0289
EVVDYLGIPASARPV

SEQ ID NO: 574 - protein, 15 aa, synthetic construct
  REGION 1..15: note = Synthetic: Rv2823c
FDHEFTFGWDELLSK

SEQ ID NO: 575 - protein, 15 aa, synthetic construct
  REGION 1..15: note = Synthetic: Rv2823c
FDREFTFGWDELLSK

SEQ ID NO: 576 - protein, 15 aa, synthetic construct
  REGION 1..15: note = Synthetic: Rv2608
FGFSIPQLGFTLSGA

SEQ ID NO: 577 - protein, 15 aa, synthetic construct
  REGION 1..15: note = Synthetic: Rv2608
FGLFPDVDWAEVAAD

SEQ ID NO: 578 - protein, 15 aa, synthetic construct
  REGION 1..15: note = Synthetic: Rv1705c
FGQNTASIAATEAQY

SEQ ID NO: 579 - protein, 15 aa, synthetic construct
  REGION 1..15: note = Synthetic: Rv1802
FGQNTGAIAAAEARY

SEQ ID NO: 580 - protein, 15 aa, synthetic construct
  REGION 1..15: note = Synthetic: Rv1789
FGQNTSAIAAAEAQY

SEQ ID NO: 581 - protein, 15 aa, synthetic construct
  REGION 1..15: note = Synthetic: Rv1199c
FPDRASIIRLVGAVL

SEQ ID NO: 582 - protein, 15 aa, synthetic construct
  REGION 1..15: note = Synthetic: Rv3619
FQVIYEQANAHGQKV

SEQ ID NO: 583 - protein, 15 aa, synthetic construct
  REGION 1..15: note = Synthetic: Rv2853
FVQALTTAAASYASV

SEQ ID NO: 584 - protein, 15 aa, synthetic construct
  REGION 1..15: note = Synthetic: Rv2823c
FYNEKAFLLTTFDVS

SEQ ID NO: 585 - protein, 15 aa, synthetic construct
  REGION 1..15: note = Synthetic: Rv2024c
GADATAAAAFEQFLA

SEQ ID NO: 586 - protein, 15 aa, synthetic construct
  REGION 1..15: note = Synthetic: Rv0453
GAMVATNFFGINTIP

SEQ ID NO: 587 - protein, 15 aa, synthetic construct
  REGION 1..15: note = Synthetic: Rv1886c
GCQTYKWETFLTSEL

SEQ ID NO: 588 - protein, 15 aa, synthetic construct
  REGION 1..15: note = Synthetic: Rv3418c
GEEYLILSARDVLAV

SEQ ID NO: 589 - protein, 15 aa, synthetic construct
  REGION 1..15: note = Synthetic: Rv3804c
GGHNGVFDFPDSGTH

SEQ ID NO: 590 - protein, 15 aa, synthetic construct
  REGION 1..15: note = Synthetic: Rv1196
GGLPVGQMGARAGGG

SEQ ID NO: 591 - protein, 15 aa, synthetic construct
  REGION 1..15: note = Synthetic: Rv3804c
GGQSSFYSDWYQPAC

SEQ ID NO: 592 - protein, 15 aa, synthetic construct
  REGION 1..15: note = Synthetic: Rv2608
GILTRFGFSIPQLGF

SEQ ID NO: 593 - protein, 15 aa, synthetic construct
  REGION 1..15: note = Synthetic: Rv3804c
GKAGCQTYKWETFLT

SEQ ID NO: 594 - protein, 15 aa, synthetic construct
  REGION 1..15: note = Synthetic: Rv2608
GLAGDAWHGPASLAM

SEQ ID NO: 595 - protein, 15 aa, synthetic construct
  REGION 1..15: note = Synthetic: Rv3874
GNFERISGDLKTQID

SEQ ID NO: 596 - protein, 15 aa, synthetic construct
  REGION 1..15: note = Synthetic: Rv0289
GNGVVALRNAQLVTF

SEQ ID NO: 597 - protein, 15 aa, synthetic construct
  REGION 1..15: note = Synthetic: Rv0129c
GQNYTYKWETFLTRE

SEQ ID NO: 598 - protein, 15 aa, synthetic construct
  REGION 1..15: note = Synthetic: Rv3874
GQWRGAAGTAAQAAV

SEQ ID NO: 599 - protein, 15 aa, synthetic construct
  REGION 1..15: note = Synthetic: Rv2874
GTVVLTATFALGAAL

SEQ ID NO: 600 - protein, 15 aa, synthetic construct
  REGION 1..15: note = Synthetic: Rv3021c
GWIISNIFGAIPVLA

SEQ ID NO: 601 - protein, 15 aa, synthetic construct
  REGION 1..15: note = Synthetic: Rv3021c
GWIISNIFGAIPVLG

SEQ ID NO: 602 - protein, 15 aa, synthetic construct
  REGION 1..15: note = Synthetic: Rv0453
GWSSLGREYAAVAEE

SEQ ID NO: 603 - protein, 15 aa, synthetic construct
  REGION 1..15: note = Synthetic: Rv3619
HGQKVQAAGNNMAQT

SEQ ID NO: 604 - protein, 15 aa, synthetic construct
  REGION 1..15: note = Synthetic: Rv1886c
HPQQFIYAGSLSALL

SEQ ID NO: 605 - protein, 15 aa, synthetic construct
  REGION 1..15: note = Synthetic: Rv1196
IAENRAELMILIATN

SEQ ID NO: 606 - protein, 15 aa, synthetic construct
  REGION 1..15: note = Synthetic: Rv0985c
IDLNVLLSAAINFFL

SEQ ID NO: 607 - protein, 15 aa, synthetic construct
  REGION 1..15: note = Synthetic: Rv0987
IGLVTQTINDFYFVI

SEQ ID NO: 608 - protein, 15 aa, synthetic construct
  REGION 1..15: note = Synthetic: Rv3025c
ILPIAEMSVVAMEFG

SEQ ID NO: 609 - protein, 15 aa, synthetic construct
  REGION 1..15: note = Synthetic: Rv2024c
IMLLAYYIAAVNIES

SEQ ID NO: 610 - protein, 15 aa, synthetic construct
  REGION 1..15: note = Synthetic: Rv2996c
INLIIHYVDRPGALG

SEQ ID NO: 611 - protein, 15 aa, synthetic construct
  REGION 1..15: note = Synthetic: Rv2996c
INLIIHYVHRAGALG

SEQ ID NO: 612 - protein, 15 aa, synthetic construct
  REGION 1..15: note = Synthetic: Rv3136
IQARAAALAFEQAYA

SEQ ID NO: 613 - protein, 15 aa, synthetic construct
  REGION 1..15: note = Synthetic: Rv1317c
IRQLERLLQAVVGAG

SEQ ID NO: 614 - protein, 15 aa, synthetic construct
  REGION 1..15: note = Synthetic: Rv2996c
IVQINGRHFDLRAQG

SEQ ID NO: 615 - protein, 15 aa, synthetic construct
  REGION 1..15: note = Synthetic: Rv3874
KTQIDQVESTAGSLQ

SEQ ID NO: 616 - protein, 15 aa, synthetic construct
  REGION 1..15: note = Synthetic: Rv1808
LAAAAAWDALAAELY

SEQ ID NO: 617 - protein, 15 aa, synthetic construct
  REGION 1..15: note = Synthetic: Rv2874
LALVGFLGGLITGIS

SEQ ID NO: 618 - protein, 15 aa, synthetic construct
  REGION 1..15: note = Synthetic: Rv2874
LALVGFLGGLITGTS

SEQ ID NO: 619 - protein, 15 aa, synthetic construct
  REGION 1..15: note = Synthetic: Rv3874
LAQEAGNFERISGDL

SEQ ID NO: 620 - protein, 15 aa, synthetic construct
  REGION 1..15: note = Synthetic: Rv1196
LAQPTQGTTPSSKLG

SEQ ID NO: 621 - protein, 15 aa, synthetic construct
  REGION 1..15: note = Synthetic: Rv0256c
LAWLVQASANSAAMA

SEQ ID NO: 622 - protein, 15 aa, synthetic construct
  REGION 1..15: note = Synthetic: Rv0293c
LDYLRRMTVFLQGLM

SEQ ID NO: 623 - protein, 15 aa, synthetic construct
  REGION 1..15: note = Synthetic: Rv3330
LENDNQLLYNYPGAL

SEQ ID NO: 624 - protein, 15 aa, synthetic construct
  REGION 1..15: note = Synthetic: Rv1196
LGGLWTAVSPHLSPL

SEQ ID NO: 625 - protein, 15 aa, synthetic construct
  REGION 1..15: note = Synthetic: Rv2608
LGLPPPWQPALPRLF

SEQ ID NO: 626 - protein, 15 aa, synthetic construct
  REGION 1..15: note = Synthetic: Rv3620c
LHGVRDGLVRDANNY

SEQ ID NO: 627 - protein, 15 aa, synthetic construct
  REGION 1..15: note = Synthetic: Rv3021c
LLEFAVVLELAILSI

SEQ ID NO: 628 - protein, 15 aa, synthetic construct
  REGION 1..15: note = Synthetic: Rv1196
LLEQAAAVEEASDTA

SEQ ID NO: 629 - protein, 15 aa, synthetic construct
  REGION 1..15: note = Synthetic: Rv1196
LLGQNTPAIAVNEAE

SEQ ID NO: 630 - protein, 15 aa, synthetic construct
  REGION 1..15: note = Synthetic: Rv0293c
LNYRPLLPKDRRMII

SEQ ID NO: 631 - protein, 15 aa, synthetic construct
  REGION 1..15: note = Synthetic: Rv2608
LPPEVNSARIFAGAG

SEQ ID NO: 632 - protein, 15 aa, synthetic construct
  REGION 1..15: note = Synthetic: Rv3019c
LQSLGADIASEQAVL

SEQ ID NO: 633 - protein, 15 aa, synthetic construct
  REGION 1..15: note = Synthetic: Rv1366
LQSLWANFYELLADA

SEQ ID NO: 634 - protein, 15 aa, synthetic construct
  REGION 1..15: note = Synthetic: Rv3115
LRGLLSTFIAALMGA

SEQ ID NO: 635 - protein, 15 aa, synthetic construct
  REGION 1..15: note = Synthetic: Rv1886c
LSIVMPVGGQSSFYS

SEQ ID NO: 636 - protein, 15 aa, synthetic construct
  REGION 1..15: note = Synthetic: Rv1196
LSPISNMVSMANNHM

SEQ ID NO: 637 - protein, 15 aa, synthetic construct
  REGION 1..15: note = Synthetic: Rv1196
LSPISNMVSMANNHV

SEQ ID NO: 638 - protein, 15 aa, synthetic construct
  REGION 1..15: note = Synthetic: Rv1196
LSPLSNMVSMANNHM

SEQ ID NO: 639 - protein, 15 aa, synthetic construct
  REGION 1..15: note = Synthetic: Rv3875
LTKLAAAWGGSGSEA

SEQ ID NO: 640 - protein, 15 aa, synthetic construct
  REGION 1..15: note = Synthetic: Rv1886c
LTSELPQWLSANRAV

SEQ ID NO: 641 - protein, 15 aa, synthetic construct
  REGION 1..15: note = Synthetic: Rv2608
LTTYILLPSQDLPLL

SEQ ID NO: 642 - protein, 15 aa, synthetic construct
  REGION 1..15: note = Synthetic: Rv3330
MAFLRSVSCLAAAVF

SEQ ID NO: 643 - protein, 15 aa, synthetic construct
  REGION 1..15: note = Synthetic: Rv3330
MAFLRSVSRLAAAVF

SEQ ID NO: 644 - protein, 15 aa, synthetic construct
  REGION 1..15: note = Synthetic: Rv1195
MHVSFVMAYPEMLAA

SEQ ID NO: 645 - protein, 15 aa, synthetic construct
  REGION 1..15: note = Synthetic: Rv0288
MLGHAGDMAGYAGTL

SEQ ID NO: 646 - protein, 15 aa, synthetic construct
  REGION 1..15: note = Synthetic: Rv2608
MNFAVLPPEVNSARI

SEQ ID NO: 647 - protein, 15 aa, synthetic construct
  REGION 1..15: note = Synthetic: Rv1788
MSFVTTQPEALAAAA

SEQ ID NO: 648 - protein, 15 aa, synthetic construct
  REGION 1..15: note = Synthetic: Rv3620c
MTDPHAMRDMAGRFE

SEQ ID NO: 649 - protein, 15 aa, synthetic construct
  REGION 1..15: note = Synthetic: Rv2346c
MTINYQFGDVDAHGA

SEQ ID NO: 650 - protein, 15 aa, synthetic construct
  REGION 1..15: note = Synthetic: Rv3620c
MTSRFMTDPHAMRDM

SEQ ID NO: 651 - protein, 15 aa, synthetic construct
  REGION 1..15: note = Synthetic: Rv1196
MVAAASPYVAWMSVT

SEQ ID NO: 652 - protein, 15 aa, synthetic construct
  REGION 1..15: note = Synthetic: Rv1196
MVDFGALPPEINSAR

SEQ ID NO: 653 - protein, 15 aa, synthetic construct
  REGION 1..15: note = Synthetic: Rv0294
MWDPDVYLAFSGHRN

SEQ ID NO: 654 - protein, 15 aa, synthetic construct
  REGION 1..15: note = Synthetic: Rv0690c
MYRELLELVAADVES

SEQ ID NO: 655 - protein, 15 aa, synthetic construct
  REGION 1..15: note = Synthetic: Rv3620c
NIVNMLHGVRDGLVR

SEQ ID NO: 656 - protein, 15 aa, synthetic construct
  REGION 1..15: note = Synthetic: Rv1789
NRASLMQLISTNVFG

SEQ ID NO: 657 - protein, 15 aa, synthetic construct
  REGION 1..15: note = Synthetic: Rv1196
PGQMLGGLPVGQMGA

SEQ ID NO: 658 - protein, 15 aa, synthetic construct
  REGION 1..15: note = Synthetic: Rv2608
PQLGFTLSGATPADA

SEQ ID NO: 659 - protein, 15 aa, synthetic construct
  REGION 1..15: note = Synthetic: Rv0125
PQVVNINTKLGYNNA

SEQ ID NO: 660 - protein, 15 aa, synthetic construct
  REGION 1..15: note = Synthetic: Rv1886c
PQWLSANRAVKPTGS

SEQ ID NO: 661 - protein, 15 aa, synthetic construct
  REGION 1..15: note = Synthetic: Rv3804c
PSPSMGRDIKVQFQS

SEQ ID NO: 662 - protein, 15 aa, synthetic construct
  REGION 1..15: note = Synthetic: Rv1886c
PVGGQSSFYSDWYSP

SEQ ID NO: 663 - protein, 15 aa, synthetic construct
  REGION 1..15: note = Synthetic: Rv3619
QAAGNNMAQTDSAVG

SEQ ID NO: 664 - protein, 15 aa, synthetic construct
  REGION 1..15: note = Synthetic: Rv3875
QAMASTEGNVTGMFA

SEQ ID NO: 665 - protein, 15 aa, synthetic construct
  REGION 1..15: note = Synthetic: Rv3115
QASPDLLRGLLSTFI

SEQ ID NO: 666 - protein, 15 aa, synthetic construct
  REGION 1..15: note = Synthetic: Rv3619
QFGDVDAHGAMIRAQ

SEQ ID NO: 667 - protein, 15 aa, synthetic construct
  REGION 1..15: note = Synthetic: Rv3619
QLGRNFQVIYEQANA

SEQ ID NO: 668 - protein, 15 aa, synthetic construct
  REGION 1..15: note = Synthetic: Rv2031c
QMATTLPVQRHPRSL

SEQ ID NO: 669 - protein, 15 aa, synthetic construct
  REGION 1..15: note = Synthetic: Rv3024c
QQIKFAALSARAVAL

SEQ ID NO: 670 - protein, 15 aa, synthetic construct
  REGION 1..15: note = Synthetic: Rv2660c
QRNLSVVAPSQFTFS

SEQ ID NO: 671 - protein, 15 aa, synthetic construct
  REGION 1..15: note = Synthetic: Rv3804c
QTYKWETFLTSELPG

SEQ ID NO: 672 - protein, 15 aa, synthetic construct
  REGION 1..15: note = Synthetic: Rv0129c
QVPSASMGRDIKVQF

SEQ ID NO: 673 - protein, 15 aa, synthetic construct
  REGION 1..15: note = Synthetic: Rv0293c
RCALHWFPGSHLLAC

SEQ ID NO: 674 - protein, 15 aa, synthetic construct
  REGION 1..15: note = Synthetic: Rv0293c
RCALHWFPGSHLLHV

SEQ ID NO: 675 - protein, 15 aa, synthetic construct
  REGION 1..15: note = Synthetic: Rv2874
RGKVVLIDFWAYPCI

SEQ ID NO: 676 - protein, 15 aa, synthetic construct
  REGION 1..15: note = Synthetic: Rv3876
RQSGATIADVLAEKE

SEQ ID NO: 677 - protein, 15 aa, synthetic construct
  REGION 1..15: note = Synthetic: Rv3620c
RRMWASAQNISGAGW

SEQ ID NO: 678 - protein, 15 aa, synthetic construct
  REGION 1..15: note = Synthetic: Rv1196
RSPISNMVSMANNHM

SEQ ID NO: 679 - protein, 15 aa, synthetic construct
  REGION 1..15: note = Synthetic: Rv1199c
RSRPRRTTRRMDRRT

SEQ ID NO: 680 - protein, 15 aa, synthetic construct
  REGION 1..15: note = Synthetic: Rv0299
RVPEDLLAMVVAVEQ

SEQ ID NO: 681 - protein, 15 aa, synthetic construct
  REGION 1..15: note = Synthetic: Rv1886c
SAMILAAYHPQQFIY

SEQ ID NO: 682 - protein, 15 aa, synthetic construct
  REGION 1..15: note = Synthetic: Rv3620c
SAQNISGAGWSGMAE

SEQ ID NO: 683 - protein, 15 aa, synthetic construct
  REGION 1..15: note = Synthetic: Rv3025c
SARLRLLRDRLVEGV

SEQ ID NO: 684 - protein, 15 aa, synthetic construct
  REGION 1..15: note = Synthetic: Rv2608
SPAMVAANRTRLASL

SEQ ID NO: 685 - protein, 15 aa, synthetic construct
  REGION 1..15: note = Synthetic: Rv1196
SPYVAWMSVTAGQAE

SEQ ID NO: 686 - protein, 15 aa, synthetic construct
  REGION 1..15: note = Synthetic: Rv1886c
SSFYSDWYSPACGKA

SEQ ID NO: 687 - protein, 15 aa, synthetic construct
  REGION 1..15: note = Synthetic: Rv1195
SSYAATEVANAAAAS

SEQ ID NO: 688 - protein, 15 aa, synthetic construct
  REGION 1..15: note = Synthetic: Rv1195
SSYAATEVANAAAGQ

SEQ ID NO: 689 - protein, 15 aa, synthetic construct
  REGION 1..15: note = Synthetic: Rv0294
STIFPFRRLFMVADV

SEQ ID NO: 690 - protein, 15 aa, synthetic construct
  REGION 1..15: note = Synthetic: Rv0294
STIFPFRRLFMVAEV

SEQ ID NO: 691 - protein, 15 aa, synthetic construct
  REGION 1..15: note = Synthetic: Rv0125
SVLAAVGLGLATAPA

SEQ ID NO: 692 - protein, 15 aa, synthetic construct
  REGION 1..15: note = Synthetic: Rv3875
TATELNNALQNLART

SEQ ID NO: 693 - protein, 15 aa, synthetic construct
  REGION 1..15: note = Synthetic: Rv3874
TDAATLAQEAGNFER

SEQ ID NO: 694 - protein, 15 aa, synthetic construct
  REGION 1..15: note = Synthetic: Rv1886c
THSWEYWGAQLNAMK

SEQ ID NO: 695 - protein, 15 aa, synthetic construct
  REGION 1..15: note = Synthetic: Rv2608
TRAASPYVGWLNTAA

SEQ ID NO: 696 - protein, 15 aa, synthetic construct
  REGION 1..15: note = Synthetic: Rv1196
VAAAQMWDSVASDLF

SEQ ID NO: 697 - protein, 15 aa, synthetic construct
  REGION 1..15: note = Synthetic: Rv0125
VAQVGPQVVNINTKL

SEQ ID NO: 698 - protein, 15 aa, synthetic construct
  REGION 1..15: note = Synthetic: Rv3620c
VEDEARRMWASAQNI

SEQ ID NO: 699 - protein, 15 aa, synthetic construct
  REGION 1..15: note = Synthetic: Rv3804c
VEYLQVPSPSMGRDI

SEQ ID NO: 700 - protein, 15 aa, synthetic construct
  REGION 1..15: note = Synthetic: Rv3620c
VHAQTVEDEARRMWA

SEQ ID NO: 701 - protein, 15 aa, synthetic construct
  REGION 1..15: note = Synthetic: Rv1196
VPPPVIAENRAELMI

SEQ ID NO: 702 - protein, 15 aa, synthetic construct
  REGION 1..15: note = Synthetic: Rv3804c
VPSPSMGRDIKVQFQ

SEQ ID NO: 703 - protein, 15 aa, synthetic construct
  REGION 1..15: note = Synthetic: Rv2608
VQGVNDALSGLGLPP

SEQ ID NO: 704 - protein, 15 aa, synthetic construct
  REGION 1..15: note = Synthetic: Rv3874
VQYSRADEEQQQALS

SEQ ID NO: 705 - protein, 15 aa, synthetic construct
  REGION 1..15: note = Synthetic: Rv3874
VRFQEAANKQKQELD

SEQ ID NO: 706 - protein, 15 aa, synthetic construct
  REGION 1..15: note = Synthetic: Rv3136
VTANRAELKALIASN

SEQ ID NO: 707 - protein, 15 aa, synthetic construct
  REGION 1..15: note = Synthetic: Rv0298
VTVDAAVLAAIDADA

SEQ ID NO: 708 - protein, 15 aa, synthetic construct
  REGION 1..15: note = Synthetic: Rv2660c
VVAPSQFTFSSRSPD

SEQ ID NO: 709 - protein, 15 aa, synthetic construct
  REGION 1..15: note = Synthetic: Rv1705c
WFINWYLPISQLFYN

SEQ ID NO: 710 - protein, 15 aa, synthetic construct
  REGION 1..15: note = Synthetic: Rv1196
WMSVTAGQAELTAAQ

SEQ ID NO: 711 - protein, 15 aa, synthetic construct
  REGION 1..15: note = Synthetic: Rv3875
WNFAGIEAAASAIQG

SEQ ID NO: 712 - protein, 15 aa, synthetic construct
  REGION 1..15: note = Synthetic: Rv0453
YAAALVAMPTLAELA

SEQ ID NO: 713 - protein, 15 aa, synthetic construct
  REGION 1..15: note = Synthetic: Rv2853
YASVEAANASPLQVA

SEQ ID NO: 714 - protein, 15 aa, synthetic construct
  REGION 1..15: note = Synthetic: Rv1196
YGEMWAQDAAAMFGY

SEQ ID NO: 715 - protein, 15 aa, synthetic construct
  REGION 1..15: note = Synthetic: Rv3115
YLGLEVLTRARAALT

SEQ ID NO: 716 - protein, 15 aa, synthetic construct
  REGION 1..15: note = Synthetic: Rv2608
YPTVDYAFQYDGVND

SEQ ID NO: 717 - protein, 15 aa, synthetic construct
  REGION 1..15: note = Synthetic: Rv3015c
YRIAARPGAVTRRAA

SEQ ID NO: 718 - protein, 15 aa, synthetic construct
  REGION 1..15: note = Synthetic: Rv1802
YVAWMSATAALAREA

SEQ ID NO: 719 - protein, 15 aa, synthetic construct
  REGION 1..15: note = Synthetic: Rv1038c, Rv1197, Rv1792, Rv2347c, Rv3620c
NQAFRNIVNMLHGVR

SEQ ID NO: 720 - protein, 15 aa, synthetic construct
  REGION 1..15: note = Synthetic: Rv1196, Rv1361c, Rv3478
INSARMYAGPGSASL

SEQ ID NO: 721 - protein, 15 aa, synthetic construct
  REGION 1..15: note = Synthetic: Rv0107c
DQLVRRVVVPAVATT

SEQ ID NO: 722 - protein, 15 aa, synthetic construct
  REGION 1..15: note = Synthetic: Rv0287
AAFQAAHARFVAAAA

SEQ ID NO: 723 - protein, 15 aa, synthetic construct
  REGION 1..15: note = Synthetic: Rv0288
AMMARDTAEAAKWGG

SEQ ID NO: 724 - protein, 15 aa, synthetic construct
  REGION 1..15: note = Synthetic: Rv0288
AQWNQAMEDLVRAYH

SEQ ID NO: 725 - protein, 15 aa, synthetic construct
  REGION 1..15: note = Synthetic: Rv0288
EDLVRAYHSMSSTHE

SEQ ID NO: 726 - protein, 15 aa, synthetic construct
  REGION 1..15: note = Synthetic: Rv0288
MAMMARDTAEAAKWG

SEQ ID NO: 727 - protein, 15 aa, synthetic construct
  REGION 1..15: note = Synthetic: Rv0288, Rv3019c
GDMAGYAGTLQSLGA

SEQ ID NO: 728 - protein, 15 aa, synthetic construct
  REGION 1..15: note = Synthetic: Rv0676c
ALCLIFIIMLIITRS

SEQ ID NO: 729 - protein, 15 aa, synthetic construct
  REGION 1..15: note = Synthetic: Rv1196, Rv1361c
LIATNLLGQNTPAIA

SEQ ID NO: 730 - protein, 15 aa, synthetic construct
  REGION 1..15: note = Synthetic: Rv1196, Rv1361c
SSAGLMVAAASPYVA

SEQ ID NO: 731 - protein, 15 aa, synthetic construct
  REGION 1..15: note = Synthetic: Rv1387, Rv3018c, Rv3021c
AVLVATNFFGINTIP

SEQ ID NO: 732 - protein, 15 aa, synthetic construct
  REGION 1..15: note = Synthetic: Rv3019c
MSQIMYNYPAMMAHA

SEQ ID NO: 733 - protein, 15 aa, synthetic construct
  REGION 1..15: note = Synthetic: Rv3136
AAGGWDSLAAELATT

SEQ ID NO: 734 - protein, 15 aa, synthetic construct
  REGION 1..15: note = Synthetic: Rv3615c
LRIAAKIYSEADEAW

SEQ ID NO: 735 - protein, 15 aa, synthetic construct
  REGION 1..15: note = Synthetic: Rv3874
EISTNIRQAGVQYSR

SEQ ID NO: 736 - protein, 15 aa, synthetic construct
  REGION 1..15: note = Synthetic: Rv3874
IRQAGVQYSRADEEQ

SEQ ID NO: 737 - protein, 15 aa, synthetic construct
  REGION 1..15: note = Synthetic: Rv3874
KQELDEISTNIRQAG

SEQ ID NO: 738 - protein, 15 aa, synthetic construct
  REGION 1..15: note = Synthetic: Rv3875
ISEAGQAMASTEGNV

SEQ ID NO: 739 - protein, 15 aa, synthetic construct
  REGION 1..15: note = Synthetic: Rv3875
MTEQQWNFAGIEAAA

SEQ ID NO: 740 - protein, 15 aa, synthetic construct
  REGION 1..15: note = Synthetic: Rv3875
NLARTISEAGQAMAS

SEQ ID NO: 741 - protein, 15 aa, synthetic construct
  REGION 1..15: note = Synthetic: Rv3875
QKWDATATELNNALQ

SEQ ID NO: 742 - protein, 15 aa, synthetic construct
  REGION 1..15: note = Synthetic: Rv3875
YQGVQQKWDATATEL

SEQ ID NO: 743 - protein, 15 aa, synthetic construct
  REGION 1..15: note = Synthetic: Rv3887c
ATRFRPIIRLTVEWL

SEQ ID NO: 744 - protein, 15 aa, synthetic construct
  REGION 1..15: note = Synthetic: Rv0016c
ADGLRADPRNQRVLL

SEQ ID NO: 745 - protein, 15 aa, synthetic construct
  REGION 1..15: note = Synthetic: Rv0017c
GLTAILMLYSIVIIR

SEQ ID NO: 746 - protein, 15 aa, synthetic construct
  REGION 1..15: note = Synthetic: Rv0040c
DSQKLLAWQTTNASM

SEQ ID NO: 747 - protein, 15 aa, synthetic construct
  REGION 1..15: note = Synthetic: Rv0144
ADAQYPASTAFLATT

SEQ ID NO: 748 - protein, 15 aa, synthetic construct
  REGION 1..15: note = Synthetic: Rv0160c
GGLLARFPGFYIPFL

SEQ ID NO: 749 - protein, 15 aa, synthetic construct
  REGION 1..15: note = Synthetic: Rv0174
LILFAIVSVVAIVVL

SEQ ID NO: 750 - protein, 15 aa, synthetic construct
  REGION 1..15: note = Synthetic: Rv01763
ACLLGLTILLLAVNR

SEQ ID NO: 751 - protein, 15 aa, synthetic construct
  REGION 1..15: note = Synthetic: Rv01763
IRRLTAAVALAAAGA

SEQ ID NO: 752 - protein, 15 aa, synthetic construct
  REGION 1..15: note = Synthetic: Rv0205
AAVLVLFTLIFFLYG

SEQ ID NO: 753 - protein, 15 aa, synthetic construct
  REGION 1..15: note = Synthetic: Rv0235c
QLYQYRFTTVAELRR

SEQ ID NO: 754 - protein, 15 aa, synthetic construct
  REGION 1..15: note = Synthetic: Rv0244c
NVRDQVFNLFEVLGV

SEQ ID NO: 755 - protein, 15 aa, synthetic construct
  REGION 1..15: note = Synthetic: Rv0266c
YLRIAVANMANAVKK

SEQ ID NO: 756 - protein, 15 aa, synthetic construct
  REGION 1..15: note = Synthetic: Rv0267
LSRAGLFFVPLAVAA

SEQ ID NO: 757 - protein, 15 aa, synthetic construct
  REGION 1..15: note = Synthetic: Rv0268c
ISAERYESLMEELED

SEQ ID NO: 758 - protein, 15 aa, synthetic construct
  REGION 1..15: note = Synthetic: Rv0276
ESPVSYHAFDPELQL

SEQ ID NO: 759 - protein, 15 aa, synthetic construct
  REGION 1..15: note = Synthetic: Rv0276
FNLMLWDLDRRMRRG

SEQ ID NO: 760 - protein, 15 aa, synthetic construct
  REGION 1..15: note = Synthetic: Rv0276
YLYPIVALRIRGIAL

SEQ ID NO: 761 - protein, 15 aa, synthetic construct
  REGION 1..15: note = Synthetic: Rv0280
GVLVATNFFGINTIP

SEQ ID NO: 762 - protein, 15 aa, synthetic construct
  REGION 1..15: note = Synthetic: Rv0284
AGHTHYLIIDDVDQV

SEQ ID NO: 763 - protein, 15 aa, synthetic construct
  REGION 1..15: note = Synthetic: Rv0284
EVFLVIDNLYGFGRD

SEQ ID NO: 764 - protein, 15 aa, synthetic construct
  REGION 1..15: note = Synthetic: Rv0284
FFPFVLLLAATALYR

SEQ ID NO: 765 - protein, 15 aa, synthetic construct
  REGION 1..15: note = Synthetic: Rv0284
GILIVGMIVALVATG

SEQ ID NO: 766 - protein, 15 aa, synthetic construct
  REGION 1..15: note = Synthetic: Rv0284
ISPQTLFFPFVLLLA

SEQ ID NO: 767 - protein, 15 aa, synthetic construct
  REGION 1..15: note = Synthetic: Rv0284
LIIDDVDQVPDSPAM

SEQ ID NO: 768 - protein, 15 aa, synthetic construct
  REGION 1..15: note = Synthetic: Rv0287
AAGTYVAADAAAAST

SEQ ID NO: 769 - protein, 15 aa, synthetic construct
  REGION 1..15: note = Synthetic: Rv0288
AMEDLVRAYHAMSST

SEQ ID NO: 770 - protein, 15 aa, synthetic construct
  REGION 1..15: note = Synthetic: Rv0288
DTGITYQAWQAQWNQ

SEQ ID NO: 771 - protein, 15 aa, synthetic construct
  REGION 1..15: note = Synthetic: Rv0288
EDLVRAYHAMSSTHE

SEQ ID NO: 772 - protein, 15 aa, synthetic construct
  REGION 1..15: note = Synthetic: Rv0288
HEANTMAMMARDTAE

SEQ ID NO: 773 - protein, 15 aa, synthetic construct
  REGION 1..15: note = Synthetic: Rv0288
LQSLGAEIAVEQAAL

SEQ ID NO: 774 - protein, 15 aa, synthetic construct
  REGION 1..15: note = Synthetic: Rv0288
MSQIMYNYPAMLGHA

SEQ ID NO: 775 - protein, 15 aa, synthetic construct
  REGION 1..15: note = Synthetic: Rv0288
QAALQSAWQGDTGIT

SEQ ID NO: 776 - protein, 15 aa, synthetic construct
  REGION 1..15: note = Synthetic: Rv0288
SAWQGDTGITYQAWQ

SEQ ID NO: 777 - protein, 15 aa, synthetic construct
  REGION 1..15: note = Synthetic: Rv0288
VRAYHAMSSTHEANT

SEQ ID NO: 778 - protein, 15 aa, synthetic construct
  REGION 1..15: note = Synthetic: Rv0288
YAGTLQSLGAEIAVE

SEQ ID NO: 779 - protein, 15 aa, synthetic construct
  REGION 1..15: note = Synthetic: Rv0288
YNYPAMLGHAGDMAG

SEQ ID NO: 780 - protein, 15 aa, synthetic construct
  REGION 1..15: note = Synthetic: Rv0288
YQAWQAQWNQAMEDL

SEQ ID NO: 781 - protein, 15 aa, synthetic construct
  REGION 1..15: note = Synthetic: Rv0290
GWYLVAATAAAATLR

SEQ ID NO: 782 - protein, 15 aa, synthetic construct
  REGION 1..15: note = Synthetic: Rv0290
IPVMAYLVGLFAWVL

SEQ ID NO: 783 - protein, 15 aa, synthetic construct
  REGION 1..15: note = Synthetic: Rv0290
LPLRRLLGLVAAGLD

SEQ ID NO: 784 - protein, 15 aa, synthetic construct
  REGION 1..15: note = Synthetic: Rv0290
QLSALWARFPLPVIP

SEQ ID NO: 785 - protein, 15 aa, synthetic construct
  REGION 1..15: note = Synthetic: Rv0290
QSGFIAAAVLLSVLG

SEQ ID NO: 786 - protein, 15 aa, synthetic construct
  REGION 1..15: note = Synthetic: Rv0361
EDQVRQAIQSLDIAI

SEQ ID NO: 787 - protein, 15 aa, synthetic construct
  REGION 1..15: note = Synthetic: Rv0440
EDAVRNAKAAVEEGI

SEQ ID NO: 788 - protein, 15 aa, synthetic construct
  REGION 1..15: note = Synthetic: Rv0440
MAKTIAYDEEARRGL

SEQ ID NO: 789 - protein, 15 aa, synthetic construct
  REGION 1..15: note = Synthetic: Rv0573c
EQDLRYLRGLGQFSD

SEQ ID NO: 790 - protein, 15 aa, synthetic construct
  REGION 1..15: note = Synthetic: Rv0585c
LPFVILIGLIVSRQW

SEQ ID NO: 791 - protein, 15 aa, synthetic construct
  REGION 1..15: note = Synthetic: Rv0590A
AGGIYGDFFNFYLCD

SEQ ID NO: 792 - protein, 15 aa, synthetic construct
  REGION 1..15: note = Synthetic: Rv0694
GITVADNVAAFSELG

SEQ ID NO: 793 - protein, 15 aa, synthetic construct
  REGION 1..15: note = Synthetic: Rv0731c
FGEVIYVSAELKQKH

SEQ ID NO: 794 - protein, 15 aa, synthetic construct
  REGION 1..15: note = Synthetic: Rv0836c
RATFRALGSTGHRFL

SEQ ID NO: 795 - protein, 15 aa, synthetic construct
  REGION 1..15: note = Synthetic: Rv0876c
ELWVGFTAVSALLIL

SEQ ID NO: 796 - protein, 15 aa, synthetic construct
  REGION 1..15: note = Synthetic: Rv1005c
TTPSVLFLLLKTIAT

SEQ ID NO: 797 - protein, 15 aa, synthetic construct
  REGION 1..15: note = Synthetic: Rv1111c
LGTAAGVLLIGLVRW

SEQ ID NO: 798 - protein, 15 aa, synthetic construct
  REGION 1..15: note = Synthetic: Rv1122
GSWLLDLTAIALRES

SEQ ID NO: 799 - protein, 15 aa, synthetic construct
  REGION 1..15: note = Synthetic: Rv1173
GIGRPARQRTTTYAL

SEQ ID NO: 800 - protein, 15 aa, synthetic construct
  REGION 1..15: note = Synthetic: Rv1195
AAIHDQFVATLASSA

SEQ ID NO: 801 - protein, 15 aa, synthetic construct
  REGION 1..15: note = Synthetic: Rv1195
VHVSFVMAYPEMLAA

SEQ ID NO: 802 - protein, 15 aa, synthetic construct
  REGION 1..15: note = Synthetic: Rv1196
GSASLVAAAQMWDSV

SEQ ID NO: 803 - protein, 15 aa, synthetic construct
  REGION 1..15: note = Synthetic: Rv1196
LLPFEEAPEMTSAGG

SEQ ID NO: 804 - protein, 15 aa, synthetic construct
  REGION 1..15: note = Synthetic: Rv1196
TATATLLPFEEAPEM

SEQ ID NO: 805 - protein, 15 aa, synthetic construct
  REGION 1..15: note = Synthetic: Rv1196, Rv1361c
TPAIAVNEAEYGEMW

SEQ ID NO: 806 - protein, 15 aa, synthetic construct
  REGION 1..15: note = Synthetic: Rv1198
AEHQAIIRDVLTASD

SEQ ID NO: 807 - protein, 15 aa, synthetic construct
  REGION 1..15: note = Synthetic: Rv1198, Rv2346c
GAMIRAQAGLLEAEH

SEQ ID NO: 808 - protein, 15 aa, synthetic construct
  REGION 1..15: note = Synthetic: Rv1227c
APVMWALSASLGWIL

SEQ ID NO: 809 - protein, 15 aa, synthetic construct
  REGION 1..15: note = Synthetic: Rv13493
LIGFALLAFCSVVAR

SEQ ID NO: 810 - protein, 15 aa, synthetic construct
  REGION 1..15: note = Synthetic: Rv13513
AQHRHRYLTMVNVGR

SEQ ID NO: 811 - protein, 15 aa, synthetic construct
  REGION 1..15: note = Synthetic: Rv1387
GINTIPIAINEAEYV

SEQ ID NO: 812 - protein, 15 aa, synthetic construct
  REGION 1..15: note = Synthetic: Rv1442
KTPEWAAALSGLAAG

SEQ ID NO: 813 - protein, 15 aa, synthetic construct
  REGION 1..15: note = Synthetic: Rv1508c3
IVVVAVLALGRRIPP

SEQ ID NO: 814 - protein, 15 aa, synthetic construct
  REGION 1..15: note = Synthetic: Rv1565c
IRRLLPALVVVLAGC

SEQ ID NO: 815 - protein, 15 aa, synthetic construct
  REGION 1..15: note = Synthetic: Rv1565c
WPLLIFWLSYTGHRH

SEQ ID NO: 816 - protein, 15 aa, synthetic construct
  REGION 1..15: note = Synthetic: Rv1599
LPTVRPIVAAVAERG

SEQ ID NO: 817 - protein, 15 aa, synthetic construct
  REGION 1..15: note = Synthetic: Rv1629
FRAFYALPAENFKTR

SEQ ID NO: 818 - protein, 15 aa, synthetic construct
  REGION 1..15: note = Synthetic: Rv1681
IRLALSLGFRVRVAA

SEQ ID NO: 819 - protein, 15 aa, synthetic construct
  REGION 1..15: note = Synthetic: Rv1807, Rv1802
AAAAAYEAAFAATVP

SEQ ID NO: 820 - protein, 15 aa, synthetic construct
  REGION 1..15: note = Synthetic: Rv1807, Rv1802
AGAQAEGAAAAYEAA

SEQ ID NO: 821 - protein, 15 aa, synthetic construct
  REGION 1..15: note = Synthetic: Rv1807, Rv1802
GAAAAYEAAFAATVP

SEQ ID NO: 822 - protein, 15 aa, synthetic construct
  REGION 1..15: note = Synthetic: Rv1813c
KAWHQRTPARAEQVA

SEQ ID NO: 823 - protein, 15 aa, synthetic construct
  REGION 1..15: note = Synthetic: Rv1886c
DIKVQFQSGGNNSPA

SEQ ID NO: 824 - protein, 15 aa, synthetic construct
  REGION 1..15: note = Synthetic: Rv1886c, Rv3804c
PSMGRDIKVQFQSGG

SEQ ID NO: 825 - protein, 15 aa, synthetic construct
  REGION 1..15: note = Synthetic: Rv1888c3
ILLLVAVVALLFTGR

SEQ ID NO: 826 - protein, 15 aa, synthetic construct
  REGION 1..15: note = Synthetic: Rv1888c3
ILLLVAVVALLFTSR

SEQ ID NO: 827 - protein, 15 aa, synthetic construct
  REGION 1..15: note = Synthetic: Rv1890c
GNGYRLTGKLHIRGK

SEQ ID NO: 828 - protein, 15 aa, synthetic construct
  REGION 1..15: note = Synthetic: Rv1894c
AVGFTPEQLEIELNW

SEQ ID NO: 829 - protein, 15 aa, synthetic construct
  REGION 1..15: note = Synthetic: Rv1894c
FPIFAVTHCRDVVVA

SEQ ID NO: 830 - protein, 15 aa, synthetic construct
  REGION 1..15: note = Synthetic: Rv1997
ANRWIILGVSAQAIA

SEQ ID NO: 831 - protein, 15 aa, synthetic construct
  REGION 1..15: note = Synthetic: Rv2024c
ARILLLVPSISLLSQ

SEQ ID NO: 832 - protein, 15 aa, synthetic construct
  REGION 1..15: note = Synthetic: Rv2024c
KDDIFYYVYGLLHDP

SEQ ID NO: 833 - protein, 15 aa, synthetic construct
  REGION 1..15: note = Synthetic: Rv2024c
KQQVIAELYEKFFRI

SEQ ID NO: 834 - protein, 15 aa, synthetic construct
  REGION 1..15: note = Synthetic: Rv2031c
AYGSFVRTVSLPVGA

SEQ ID NO: 835 - protein, 15 aa, synthetic construct
  REGION 1..15: note = Synthetic: Rv2032
ESLRLYDSSYHAELF

SEQ ID NO: 836 - protein, 15 aa, synthetic construct
  REGION 1..15: note = Synthetic: Rv2040c
AILLVLTVLQLRITH

SEQ ID NO: 837 - protein, 15 aa, synthetic construct
  REGION 1..15: note = Synthetic: Rv2051c3
ILVGAAAAVVLVAMR

SEQ ID NO: 838 - protein, 15 aa, synthetic construct
  REGION 1..15: note = Synthetic: Rv2094c
TPVQSQRVDPSAASG

SEQ ID NO: 839 - protein, 15 aa, synthetic construct
  REGION 1..15: note = Synthetic: Rv2095c3
EATGLLVALRALADI

SEQ ID NO: 840 - protein, 15 aa, synthetic construct
  REGION 1..15: note = Synthetic: Rv2095c3
EATGLLVALRALANI

SEQ ID NO: 841 - protein, 15 aa, synthetic construct
  REGION 1..15: note = Synthetic: Rv2095c3
SPPGLPVAAVAEQAP

SEQ ID NO: 842 - protein, 15 aa, synthetic construct
  REGION 1..15: note = Synthetic: Rv2096c
ERLVNLVIALLSTRG

SEQ ID NO: 843 - protein, 15 aa, synthetic construct
  REGION 1..15: note = Synthetic: Rv2338c
AAEVLKILLGHGRVY

SEQ ID NO: 844 - protein, 15 aa, synthetic construct
  REGION 1..15: note = Synthetic: Rv2875
AAFSKLPASTIDELK

SEQ ID NO: 845 - protein, 15 aa, synthetic construct
  REGION 1..15: note = Synthetic: Rv3019c
MSQIMYNYPAMRAHA

SEQ ID NO: 846 - protein, 15 aa, synthetic construct
  REGION 1..15: note = Synthetic: Rv3020c
AAFQGAHARFVAAAA

SEQ ID NO: 847 - protein, 15 aa, synthetic construct
  REGION 1..15: note = Synthetic: Rv3020c
AAGTYVAADAAAASS

SEQ ID NO: 848 - protein, 15 aa, synthetic construct
  REGION 1..15: note = Synthetic: Rv30633
AIFMGCYLRFLRPGR

SEQ ID NO: 849 - protein, 15 aa, synthetic construct
  REGION 1..15: note = Synthetic: Rv3071
ARFFGRNVNVPLMII

SEQ ID NO: 850 - protein, 15 aa, synthetic construct
  REGION 1..15: note = Synthetic: Rv3125c
AAIQARAAATAFEAA

SEQ ID NO: 851 - protein, 15 aa, synthetic construct
  REGION 1..15: note = Synthetic: Rv3125c
SMLFLPVRILTSPIT

SEQ ID NO: 852 - protein, 15 aa, synthetic construct
  REGION 1..15: note = Synthetic: Rv3135
AAASWDALAAELASA

SEQ ID NO: 853 - protein, 15 aa, synthetic construct
  REGION 1..15: note = Synthetic: Rv3136
FFGQNTAAIAATEAQ

SEQ ID NO: 854 - protein, 15 aa, synthetic construct
  REGION 1..15: note = Synthetic: Rv3136
LLGQNTAAIAAIEAQ

SEQ ID NO: 855 - protein, 15 aa, synthetic construct
  REGION 1..15: note = Synthetic: Rv3148
VLMMELNRISSHLVA

SEQ ID NO: 856 - protein, 15 aa, synthetic construct
  REGION 1..15: note = Synthetic: Rv3182
RILFAFDPARQAILL

SEQ ID NO: 857 - protein, 15 aa, synthetic construct
  REGION 1..15: note = Synthetic: Rv3252c
GPILLYVLLPLLDLR

SEQ ID NO: 858 - protein, 15 aa, synthetic construct
  REGION 1..15: note = Synthetic: Rv3261
ADADIIMLAPSNPVV

SEQ ID NO: 859 - protein, 15 aa, synthetic construct
  REGION 1..15: note = Synthetic: Rv3365c
LRRNSANLLVLAGAQ

SEQ ID NO: 860 - protein, 15 aa, synthetic construct
  REGION 1..15: note = Synthetic: Rv3383c
ILLRDALHATAVRIL

SEQ ID NO: 861 - protein, 15 aa, synthetic construct
  REGION 1..15: note = Synthetic: Rv3432c
DLVWDFRLPRVVSIN

SEQ ID NO: 862 - protein, 15 aa, synthetic construct
  REGION 1..15: note = Synthetic: Rv3436c
AVLRRLEWHFTLVFA

SEQ ID NO: 863 - protein, 15 aa, synthetic construct
  REGION 1..15: note = Synthetic: Rv3458c
NVPSYRVSQYDIVDV

SEQ ID NO: 864 - protein, 15 aa, synthetic construct
  REGION 1..15: note = Synthetic: Rv3493c
GIDEISDNASVLVSV

SEQ ID NO: 865 - protein, 15 aa, synthetic construct
  REGION 1..15: note = Synthetic: Rv3522
MLMPCFAQLYDELGI

SEQ ID NO: 866 - protein, 15 aa, synthetic construct
  REGION 1..15: note = Synthetic: Rv3557c
LVYRFIRDTTWVSVR

SEQ ID NO: 867 - protein, 15 aa, synthetic construct
  REGION 1..15: note = Synthetic: Rv3615c
VDLAKSLRIAAKIYS

SEQ ID NO: 868 - protein, 15 aa, synthetic construct
  REGION 1..15: note = Synthetic: Rv36353
GWLVLIAVLALSLVR

SEQ ID NO: 869 - protein, 15 aa, synthetic construct
  REGION 1..15: note = Synthetic: Rv36353
SSWRPVLLIPLTFAL

SEQ ID NO: 870 - protein, 15 aa, synthetic construct
  REGION 1..15: note = Synthetic: Rv3645
TVIILFLAGAVVNLK

SEQ ID NO: 871 - protein, 15 aa, synthetic construct
  REGION 1..15: note = Synthetic: Rv3646c
EDALRLLSLPRVVGV

SEQ ID NO: 872 - protein, 15 aa, synthetic construct
  REGION 1..15: note = Synthetic: Rv3763
ASGPKVVIDGKDQNV

SEQ ID NO: 873 - protein, 15 aa, synthetic construct
  REGION 1..15: note = Synthetic: Rv3782
AQEWLRFGWFFLATR

SEQ ID NO: 874 - protein, 15 aa, synthetic construct
  REGION 1..15: note = Synthetic: Rv3782
AQEWLRFGWFFLVTR

SEQ ID NO: 875 - protein, 15 aa, synthetic construct
  REGION 1..15: note = Synthetic: Rv3782
NRGYVLSQPGLRKLL

SEQ ID NO: 876 - protein, 15 aa, synthetic construct
  REGION 1..15: note = Synthetic: Rv3784
DRYINWAKDQPQYPY

SEQ ID NO: 877 - protein, 15 aa, synthetic construct
  REGION 1..15: note = Synthetic: Rv3794
FCLFGVLFVLLRRGR

SEQ ID NO: 878 - protein, 15 aa, synthetic construct
  REGION 1..15: note = Synthetic: Rv3796
IVDYYNFFLSGGFLA

SEQ ID NO: 879 - protein, 15 aa, synthetic construct
  REGION 1..15: note = Synthetic: Rv3796
LHRAFAYTSNIFIRD

SEQ ID NO: 880 - protein, 15 aa, synthetic construct
  REGION 1..15: note = Synthetic: Rv3804c
MGRDIKVQFQSGGAN

SEQ ID NO: 881 - protein, 15 aa, synthetic construct
  REGION 1..15: note = Synthetic: Rv3818
GSTADFTGTTLNSLR

SEQ ID NO: 882 - protein, 15 aa, synthetic construct
  REGION 1..15: note = Synthetic: Rv3818
PDWVNTIFLSTRFRA

SEQ ID NO: 883 - protein, 15 aa, synthetic construct
  REGION 1..15: note = Synthetic: Rv3874
AAGTAAQAAVVRFQE

SEQ ID NO: 884 - protein, 15 aa, synthetic construct
  REGION 1..15: note = Synthetic: Rv3874
AEMKTDAATLAQEAG

SEQ ID NO: 885 - protein, 15 aa, synthetic construct
  REGION 1..15: note = Synthetic: Rv3874
MAEMKTDAATLAQEA

SEQ ID NO: 886 - protein, 15 aa, synthetic construct
  REGION 1..15: note = Synthetic: Rv3874
QVESTAGSLQGQWRG

SEQ ID NO: 887 - protein, 15 aa, synthetic construct
  REGION 1..15: note = Synthetic: Rv3875
NNALQNLARTISEAG

SEQ ID NO: 888 - protein, 15 aa, synthetic construct
  REGION 1..15: note = Synthetic: Rv3875
NVTSIHSLLDEGKQS

SEQ ID NO: 889 - protein, 15 aa, synthetic construct
  REGION 1..15: note = Synthetic: Rv3875
SAIQGNVTSIHSLLD

SEQ ID NO: 890 - protein, 15 aa, synthetic construct
  REGION 1..15: note = Synthetic: Rv3875
SGSEAYQGVQQKWDA

SEQ ID NO: 891 - protein, 15 aa, synthetic construct
  REGION 1..15: note = Synthetic: Rv3910
GLSSVFMAILNTRNM

SEQ ID NO: 892 - protein, 15 aa, synthetic construct
  REGION 1..15: note = Synthetic: Rv3910
GLSSVFMAILNTRNV

(19) United States
(12) Patent Application Publication
(10) Pub. No.: US 2026/0115269 A1
(43) Pub. Date: Apr. 30, 2026

(54) MYCOBACTERIUM T CELL EPITOPES, MEGAPOOLS AND USES THEREOF

(71) Applicant: La Jolla Institute for Immunology, La Jolla, CA (US)

(72) Inventors: Alessandro Sette, La Jolla, CA (US); Bjoern Peters, La Jolla, CA (US); Cecilia Lindestam Arlehamn, La Jolla, CA (US)

(21) Appl. No.: 19/112,089

(22) PCT Filed: Sep. 28, 2023
(86) PCT No.: PCT/US2023/075336

§ 371 Date: Mar. 14, 2025

Related U.S. Application Data
(60) Provisional application No. 63/411,020, filed on Sep. 28, 2022.
(60) Provisional application No. 63/458,877, filed on Apr. 12, 2023.

(51) Int. Cl. A61K 39/04; A61P 37/04; C07K 14/35; G01N 33/50; G01N 33/68

(52) U.S. Cl. CPC A61K 39/04; A61P 37/04; C07K 14/35; G01N 33/5091; G01N 33/6845

(57) ABSTRACT

The present invention includes compositions, including epitope megapools, and methods for detecting the presence of: a Mycobacterium, including a latent or active infection, or an immune response relevant to a Mycobacterium infection including T cells responsive to one or more Mycobacterium peptides or proteins from: one or more amino acid sequences selected from any one of those sequences set forth in Tables 1-5 (SEQ ID NOS: 1-718), or a subsequence, portion, homologue, variant or derivative thereof; a fusion protein; a pool of 2 or more peptides; or a polynucleotide that encodes one or more peptides or proteins, or a subsequence, portion, homologue, variant or derivative thereof. The invention further provides vaccines, diagnostics, therapies, and kits, comprising such proteins or peptides.

### CROSS-REFERENCE TO RELATED APPLICATIONS

This application is the National Stage of International Application No. PCT/US2023/075336, filed on Sep. 28, 2023, which claims priority to U.S. Provisional Application Ser. No. 63/411,020, filed Sep. 28, 2022, and 63/458,877, filed Apr. 12, 2023, the entire contents of each are incorporated herein by reference.

### STATEMENT OF GOVERNMENT SUPPORT

The inventions described in the present disclosure were made with government support under Contract No. 75N93019C00067, awarded by the National Institutes of Health. The government has certain rights in the invention.

### TECHNICAL FIELD OF THE INVENTION

The present invention relates in general to the field of proteins and peptides that are T cell epitopes and/or antigens for Mycobacterium, including epitopes and antigens from Mycobacterium tuberculosis (MTB), and more particularly, to compositions and methods for the prevention, treatment, diagnosis, kits, and uses of such T cell epitopes and antigens, including megapools, for use in detecting and characterizing Mycobacterium specific responses in infection, including latent vs active infection, and following vaccination.

### REFERENCE TO ELECTRONIC SEQUENCE LISTING

The application contains a Sequence Listing which has been submitted electronically in .XML format and is hereby incorporated by reference in its entirety. Said .XML copy, created on Oct. 27, 2023, is named “LJII2023WO.xml” and is 1,117,212 bytes in size. The sequence listing contained in this .XML file is part of the specification and is hereby incorporated by reference herein in its entirety.

### BACKGROUND OF THE INVENTION

Without limiting the scope of the invention, its background is described in connection with Mycobacterium tuberculosis.

A need remains for identifying antigens and T cell epitopes for use in diagnostics, treatments, vaccines, kits, etc., for Mycobacterium related diseases and conditions, including MTB. There is additionally a specific need in the art for optimized megapools for use in detecting and characterizing MTB specific responses in infection, including latent vs. active infection, and following vaccination.

### SUMMARY OF THE INVENTION

In one embodiment, the present invention includes a composition comprising: one or more peptides or proteins, comprising, consisting of, or consisting essentially of an amino acid sequence selected from the sequences set forth in any one of Tables 1-5 (SEQ ID NOS:1-718), or a subsequence, portion, homologue, variant or derivative thereof; a fusion protein comprising one or more amino acid sequences selected from any one of those sequences set forth in Tables 1-5 (SEQ ID NOS:1-718); a pool of 2 or more or more peptides comprising, consisting of, or consisting essentially of amino acid sequences selected from any one of those sequences set forth in Tables 1-5 (SEQ ID NOS:1-718); or a polynucleotide that encodes one or more peptides or proteins, comprising, consisting of, or consisting essentially of an amino acid sequence selected from any one of those sequences set forth in Tables 1-5 (SEQ ID NOS:1-718), or a subsequence, portion, homologue, variant or derivative thereof. In one aspect, the one or more peptides or proteins comprises, or wherein the fusion protein comprises 2 or more or more amino acid sequences selected from any one of those sequences set forth in Tables 1-5 (SEQ ID NOS:1-718), or a subsequence, portion, homologue, variant or derivative thereof. In another aspect, the amino acid sequence is selected from a Mycobacterium T cell epitope selected from any one of those sequences set forth in Tables 1-5 (SEQ ID NOS:1-718). In another aspect, the composition comprises one or more MTB peptides amino acid sequences selected from any one of those sequences set forth in Tables 1-5 (SEQ ID NOS:1-718), or a subsequence, portion, homologue, variant or derivative thereof; a fusion protein comprising one or more amino acid sequences selected from any one of those sequences set forth in Tables 1-5 (SEQ ID NOS:1-718); or a pool of 2 or more peptides selected from any one of those sequences set forth in Tables 1-5 (SEQ ID NOS:1-718). In another aspect, the peptide or protein comprises a Mycobacterium T cell epitope. In another aspect, the one or more peptides or proteins comprises a Mycobacterium CD8+ or CD4+ T cell epitope. In another aspect, the Mycobacterium is MTB and the MTB T cell epitope is not conserved in another Mycobacterium. In another aspect, the Mycobacterium is MTB and the MTB T cell epitope is conserved in another Mycobacterium. In another aspect, the one or more peptides or proteins has a length from about 9-15, 15-20, 20-25, 25-30, 30-40, 40-50, 50-75 or 75-100 amino acids. In another aspect, the one or more peptides or proteins elicits, stimulates, induces, promotes, increases or enhances a T cell response to a Mycobacterium. In another aspect, the one or more peptides or proteins that elicits, stimulates, induces, promotes, increases or enhances the T cell response to the Mycobacterium is a Mycobacterium protein or peptide, or a variant, homologue, derivative or subsequence thereof. In another aspect, the composition further comprises formulating the one or more peptides or proteins into an immunogenic formulation with an adjuvant. In another aspect, the adjuvant is selected from the group consisting of adjuvant is selected from the group consisting of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, and TLR9 ligands. In another aspect, the composition further comprises a modulator of immune response. In another aspect, the modulator of immune response is a modulator of the innate immune response. In another aspect, the modulator is Interleukin-6 (IL-6), Interferon-gamma (IFN-γ), Transforming growth factor beta (TGF-β), or Interleukin-10 (IL-10), or an agonist or antagonist thereof.

In another embodiment, the present invention includes a composition comprising monomers or multimers of: peptides or proteins comprising, consisting of, or consisting essentially of: one or more amino acid sequences selected from any one of those sequences set forth in Tables 1-5 (SEQ ID NOS:1-718), concatemers, subsequences, portions, homologues, variants or derivatives thereof; a fusion protein comprising one or more amino acid sequences selected from any one of those sequences set forth in Tables 1-5 (SEQ ID NOS:1-718); or a polynucleotide that encodes one or more peptides or proteins, comprising, consisting of, or consisting essentially of an amino acid sequence selected from any one of those sequences set forth in Tables 1-5 (SEQ ID NOS:1-718), or a subsequence, portion, homologue, variant or derivative thereof.

In another embodiment, the present invention includes a composition comprising one or more peptide-major histocompatibility complex (MHC) monomers or multimers, wherein the peptide-MHC monomer or multimer comprises a peptide comprising, consisting of, or consisting essentially of an amino acid sequence selected from any one of those sequences set forth in Tables 1-5 (SEQ ID NOS:1-718), in a groove of the MHC monomer or multimer.

In another embodiment, the present invention includes a composition comprising: one or more peptides or proteins comprising, consisting of, or consisting essentially of an amino acid sequence selected from any one of those sequences set forth in Tables 1-5 (SEQ ID NOS:1-718), or a subsequence, portion, homologue, variant or derivative thereof; a fusion protein comprising one or more amino acid sequences selected from any one of those sequences set forth in Tables 1-5 (SEQ ID NOS:1-718); a pool of 2 or more peptides selected from any one of those sequences set forth in Tables 1-5 (SEQ ID NOS:1-718); a polynucleotide that encodes one or more peptides or proteins, comprising, consisting of, or consisting essentially of an amino acid sequence selected from any one of those sequences set forth in Tables 1-5 (SEQ ID NOS:1-718), or a subsequence, portion, homologue, variant or derivative thereof. In one aspect, the one or more peptides or proteins comprises, or wherein the fusion protein comprises, 2 or more amino acid sequences selected from any one of those sequences set forth in Tables 1-5 (SEQ ID NOS:1-718), or a subsequence, portion, homologue, variant or derivative thereof. In another aspect, the protein or peptide comprises a MTB T cell epitope. In another aspect, the one or more peptides or proteins comprises a MTB CD8+ or CD4+ T cell epitope. In another aspect, the MTB T cell epitope is not conserved in another Mycobacterium. In another aspect, the MTB T cell epitope is conserved in another Mycobacterium. In another aspect, the one or more peptides or proteins has a length from about 9-15, 15-20, 20-25, 25-30, 30-40, 40-50, 50-75 or 75-100 amino acids. In another aspect, the one or more peptides or proteins elicits, stimulates, induces, promotes, increases or enhances a T cell response to MTB. In another aspect, the one or more peptides or proteins that elicits, stimulates, induces, promotes, increases or enhances the T cell response to MTB is a MTB protein or peptide, or a variant, homologue, derivative or subsequence thereof. In another aspect, the composition further comprises formulating the one or more peptides or proteins into an immunogenic formulation with an adjuvant. In another aspect, the adjuvant is selected from the group consisting of adjuvant is selected from the group consisting of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, and TLR9 ligands. In another aspect, the composition further comprises a modulator of immune response. In another aspect, the modulator of immune response is a modulator of the innate immune response. In another aspect, the modulator is Interleukin-6 (IL-6), Interferon-gamma (IFN-γ), Transforming growth factor beta (TGF-β), or Interleukin-10 (IL-10), or an agonist or antagonist thereof.

In another embodiment, the present invention includes a composition comprising monomers or multimers of: one or more peptides or proteins comprising, consisting of, or consisting essentially of: one or more MTB amino acid sequences selected from any one of those sequences set forth in Tables 1-5 (SEQ ID NOS:1-718), concatemers, subsequences, portions, homologues, variants or derivatives thereof; a fusion protein comprising one or more amino acid sequences selected from any one of those sequences set forth in Tables 1-5 (SEQ ID NOS:1-718); or a polynucleotide that encodes one or more peptides or proteins, comprising, consisting of, or consisting essentially of an amino acid sequence selected from any one of those sequences set forth in Tables 1-5 (SEQ ID NOS:1-718), or a subsequence, portion, homologue, variant or derivative thereof.

In another embodiment, the present invention includes a composition comprising one or more peptide-major histocompatibility complex (MHC) monomers or multimers, wherein the peptide-MHC monomer or multimer comprises a peptide comprising, consisting of, or consisting essentially of an amino acid sequence selected from any one of those sequences set forth in Tables 1-5 (SEQ ID NOS:1-718), in a groove of the (MHC) monomer or multimer.

In another embodiment, the present invention includes a method for detecting the presence of: (i) a Mycobacterium or (ii) an immune response relevant to Mycobacterium infections, vaccines or therapies, including T cells responsive to one or more Mycobacterium peptides, comprising: providing one or more proteins or peptides for detection of an amount or a relative amount of, and/or the activity of, and/or the state of antigen-specific T-cells; contacting a biological sample suspected of having Mycobacterium-specific T-cells to one or more proteins or peptides for detection; and detecting an amount or a relative amount of, and/or the activity of, and/or the state of antigen-specific T-cells in the biological sample, wherein the one or more proteins or peptides for detection comprise one or more amino acid sequences set forth in any one of Tables 1-5 (SEQ ID NOS:1-718), or comprise a pool of 2 or more or more amino acid sequences set forth in any one of Tables 1-5 (SEQ ID NOS:1-718). In one aspect, detecting the amount or a relative amount of, and/or activity of antigen-specific T-cells comprises one or more steps of identification or detection of the antigen-specific T-cells and measuring the amount of the antigen-specific T-cells. In another aspect, the one or more peptides or proteins comprises 2 or more amino acid sequences selected from any one of Tables 1-5 (SEQ ID NOS:1-718). In another aspect, the detecting the amount or a relative amount of, and/or activity of antigen-specific T-cells comprises indirect detection and/or direct detection. In another aspect, the method of detecting an immune response relevant to the Mycobacterium comprises the following steps: providing an MHC monomer or an MHC multimer; contacting a population T-cells to the MHC monomer or MHC multimer; and measuring the number, activity or state of T-cells specific for the MHC monomer or MHC multimer. In one aspect, the MHC monomer or MHC multimer comprises a protein or peptide of the Mycobacterium. In another aspect, the protein or peptide comprises a CD8+ or CD4+ T cell epitope. In another aspect, the T cell epitope is not conserved in another Mycobacterium. In another aspect, the T cell epitope is conserved in another Mycobacterium. In another aspect, the protein or peptide has a length from about 9-15, 15-20, 20-25, 25-30, 30-40, 40-50, 50-75 or 75-100 amino acids. In another aspect, the proteins or peptides comprise 2 or more amino acid sequences selected from any one of those sequences set forth in Tables 1-5 (SEQ ID NOS:1-718), or a subsequence, portion, homologue, variant or derivative thereof. In another aspect, the method further comprises detecting the presence or amount of the one or more peptides in a biological sample, or a response thereto, which is diagnostic of a Mycobacterium infection. In another aspect, the detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the method further comprises administering a treatment comprising the composition of one or more proteins, peptides or multimers to the subject from which the biological sample was drawn that increases the amount or relative amount of, and/or activity of the antigen-specific T-cells.

In another embodiment, the present invention includes a method for detecting the presence of: (i) MTB or (ii) an immune response relevant to MTB infections, vaccines or therapies, including T cells responsive to one or more MTB peptides, comprising: providing one or more proteins or peptides for detection of an amount or a relative amount of, and/or the activity of, and/or the state of antigen-specific T-cells; contacting a biological sample suspected of having MTB-specific T-cells to one or more proteins or peptides for detection; and detecting an amount or a relative amount of, and/or the activity of, and/or the state of antigen-specific T-cells in the biological sample, wherein the one or more proteins or peptides for detection comprise one or more amino acid sequences set forth in those sequences set forth in any one of Tables 1-5 (SEQ ID NOS:1-718), or comprise a pool of 2 or more amino acid sequences set forth in those sequences set forth in any one of Tables 1-5 (SEQ ID NOS:1-718). In one aspect, detecting the amount or a relative amount of, and/or activity of antigen-specific T-cells comprises one or more steps of identification or detection of the antigen-specific T-cells and measuring the amount of the antigen-specific T-cells. In another aspect, the one or more peptides or proteins comprises 2 or more amino acid sequences selected from any one of those sequences set forth in Tables 1-5 (SEQ ID NOS:1-718). In another aspect, detecting the amount or a relative amount of, and/or activity of antigen-specific T-cells comprises indirect detection and/or direct detection. In another aspect, detecting an immune response relevant to MTB comprises the following steps: providing an MHC monomer or an MHC multimer; contacting a population T-cells to the MHC monomer or MHC multimer; and measuring the number, activity or state of T-cells specific for the MHC monomer or MHC multimer. In another aspect, the MHC monomer or MHC multimer comprises a protein or peptide of MTB. In another aspect, the protein or peptide comprises a MTB CD8+ or CD4+ T cell epitope. In another aspect, the MTB T cell epitope is not conserved in another Mycobacterium. In another aspect, the MTB T cell epitope is conserved in another Mycobacterium. In another aspect, the protein or peptide has a length from about 9-15, 15-20, 20-25, 25-30, 30-40, 40-50, 50-75 or 75-100 amino acids. In another aspect, the proteins or peptides comprise 2 or more amino acid sequences selected from any one of those sequences set forth in Tables 1-5 (SEQ ID NOS:1-718), or a subsequence, portion, homologue, variant or derivative thereof. In another aspect, the method further comprises detecting the presence or amount of the one or more peptides in a biological sample, or a response thereto, which is diagnostic of a MTB infection, including distinguishing between latent or active MTB infection. In another aspect, detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the method further comprises administering a treatment comprising the composition of one or more proteins, peptides or multimers to the subject from which the biological sample was drawn that increases the amount or relative amount of, and/or activity of the antigen-specific T-cells.

In another embodiment, the present invention includes a method detecting a Mycobacterium infection or exposure in a subject, the method comprising, consisting of, or consisting essentially of: contacting a biological sample from a subject with a composition of composition of one or more proteins, peptides or multimers; and determining if the composition elicits an immune response from the contacted cells, wherein the presence of an immune response indicates that the subject has been exposed to or infected with Mycobacterium. In one aspect, the sample comprises T cells. In another aspect, the response comprises inducing, increasing, promoting or stimulating anti-Mycobacterium activity of T cells. In another aspect, the T cells are CD8+ or CD4+ T cells. In another aspect, the method comprises determining whether the subject has been infected by or exposed to the Mycobacterium more than once by determining if the subject elicits a secondary T cell immune response profile that is different from a primary T cell immune response profile. In another aspect, the method further comprises diagnosing a Mycobacterium infection or exposure in a subject, the method comprising contacting a biological sample from a subject with a composition of composition of one or more proteins, peptides or multimers, and determining if the composition elicits a T cell immune response, wherein the T cell immune response identifies that the subject has been infected with or exposed to a Mycobacterium. In another aspect, the method is conducted three or more days following the date of suspected infection by or exposure to a Mycobacterium.

In another embodiment, the present invention includes a method detecting MTB infection or exposure in a subject, the method comprising, consisting of, or consisting essentially of: contacting a biological sample from a subject with a composition of composition of one or more proteins, peptides or multimers; and determining if the composition elicits an immune response from the contacted cells, wherein the presence of an immune response indicates that the subject has been exposed to or infected with MTB. In another aspect, the sample comprises T cells. In another aspect, the response comprises inducing, increasing, promoting or stimulating anti-MTB activity of T cells. In another aspect, the T cells are CD8+ or CD4+ T cells. In another aspect, the method comprises determining whether the subject has been infected by or exposed to MTB more than once by determining if the subject elicits a secondary T cell immune response profile that is different from a primary T cell immune response profile. In another aspect, the method further comprises diagnosing a MTB infection or exposure in a subject, including distinguishing between latent or active MTB infection, the method comprising contacting a biological sample from a subject with a composition of one or more proteins, peptides or multimers; and determining if the composition elicits a T cell immune response, wherein the T cell immune response identifies that the subject has been infected with or exposed to MTB. In another aspect, the method is conducted three or more days following the date of suspected infection by or exposure to a Mycobacterium.

In another embodiment, the present invention includes a kit for the detection of Mycobacterium or an immune response to Mycobacterium in a subject comprising, consisting of or consisting essentially of: one or more T cells that specifically detect the presence of: one or more amino acid sequences selected from any one of those sequences set forth in Tables 1-5 (SEQ ID NOS:1-718), or a subsequence, portion, homologue, variant or derivative thereof; or a fusion protein comprising one or more amino acid sequences selected from any one of those sequences set forth in Tables 1-5 (SEQ ID NOS:1-718); or a pool of 2 or more or more peptides selected from the amino acid sequences set forth in any one of Tables 1-5 (SEQ ID NOS:1-718). In one aspect, the one or more amino acid sequences are selected from a Mycobacterium T cell epitope set forth in any one of Tables 1-5 (SEQ ID NOS:1-718). In another aspect, the composition comprises: one or more amino acid sequences selected from any one of those sequences set forth in Tables 1-5 (SEQ ID NOS:1-718), or a subsequence, portion, homologue, variant or derivative thereof; a fusion protein comprising one or more amino acid sequences selected from any one of those sequences set forth in Tables 1-5 (SEQ ID NOS:1-718); or a pool of 2 or more peptides selected from the amino acid sequences set forth in those sequences set forth in any one of Tables 1-5 (SEQ ID NOS:1-718). In another aspect, the amino acid sequence comprises a Mycobacterium CD8+ or CD4+ T cell epitope. In another aspect, the T cell epitope is not conserved in another Mycobacterium. In another aspect, the T cell epitope is conserved in another Mycobacterium. In another aspect, the fusion protein has a length from about 9-15, 15-20, 20-25, 25-30, 30-40, 40-50, 50-75 or 75-100 amino acids. In another aspect, the kit includes instruction for a diagnostic method, a process, a composition, a product, a service or component part thereof for the detection of: (i) Mycobacterium or (ii) an immune response relevant to Mycobacterium infections, vaccines or therapies, including T cells responsive to Mycobacterium. In another aspect, the kit includes reagents for detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the kit includes reagents for determining a Human Leukocyte Antigen (HLA) profile of a subject, and selecting peptides that are presented by the HLA profile of the subject for detecting an immune response to Mycobacterium.

In another embodiment, the present invention includes a kit for the detection of MTB or an immune response to MTB in a subject comprising, consisting of or consisting essentially of: one or more T cells that specifically detect the presence of: one or more amino acid sequences selected from any one of those sequences set forth in Tables 1-5 (SEQ ID NOS:1-718), or a subsequence, portion, homologue, variant or derivative thereof; a fusion protein comprising one or more amino acid sequences selected from any one of those sequences set forth in Tables 1-5 (SEQ ID NOS:1-718); or a pool of 2 or more peptides selected from the amino acid sequences set forth in those sequences set forth in any one of Tables 1-5 (SEQ ID NOS:1-718). In another aspect, the amino acid sequence comprises a MTB CD8+ or CD4+ T cell epitope. In another aspect, the MTB T cell epitope is not conserved in another Mycobacterium. In another aspect, the MTB T cell epitope is conserved in another Mycobacterium. In another aspect, the fusion protein has a length from about 9-15, 15-20, 20-25, 25-30, 30-40, 40-50, 50-75 or 75-100 amino acids. In another aspect, the kit includes instruction for a diagnostic method, a process, a composition, a product, a service or component part thereof for the detection of: (i) MTB, including distinguishing between latent or active MTB infection, or (ii) an immune response relevant to MTB infections, vaccines or therapies, including T cells responsive to MTB. In another aspect, the kit includes reagents for detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the kit includes reagents for determining a Human Leukocyte Antigen (HLA) profile of a subject, and selecting peptides that are presented by the HLA profile of the subject for detecting an immune response to MTB.

In another embodiment, the present invention includes a method of stimulating, inducing, promoting, increasing, or enhancing an immune response against a Mycobacterium in a subject, comprising: administering a composition of one or more proteins, peptides, multimers or a polynucleotide that expresses the protein, peptide or multimers, in an amount sufficient to stimulate, induce, promote, increase, or enhance an immune response against the Mycobacterium in the subject. In another aspect, the immune response provides the subject with protection against a Mycobacterium infection or pathology, or one or more physiological conditions, disorders, illnesses, diseases or symptoms caused by or associated with Mycobacterium infection or pathology. In another aspect, the immune response is specific to: one or more MTB peptides selected from the amino acid sequences set forth in any one of Tables 1-5 (SEQ ID NOS:1-718), or a subsequence, portion, homologue, variant or derivative thereof.

In another embodiment, the present invention includes a method of stimulating, inducing, promoting, increasing, or enhancing an immune response against MTB in a subject, comprising: administering a composition of proteins, peptides, multimers or a polynucleotide that expresses the protein, peptide or multimers, in an amount sufficient to stimulate, induce, promote, increase, or enhance an immune response against MTB in the subject. In one aspect, the immune response provides the subject with protection against a MTB infection or pathology, or one or more physiological conditions, disorders, illnesses, diseases or symptoms caused by or associated with MTB infection or pathology. In another aspect, the immune response is specific to: one or more MTB peptides selected from the amino acid sequences set forth in those sequences set forth in any one of Tables 1-5 (SEQ ID NOS:1-718), or a subsequence, portion, homologue, variant or derivative thereof.

In another embodiment, the present invention includes a method of stimulating, inducing, promoting, increasing, or enhancing an immune response against MTB in a subject, comprising: administering to a subject an amount of a protein or peptide comprising, consisting of or consisting essentially of an amino acid sequence of a MTB protein or peptide, or a variant, homologue, derivative or subsequence thereof, wherein the protein or peptide comprises at least two peptides selected from the amino acid sequences set forth in any one of Tables 1-5 (SEQ ID NOS:1-718) or a subsequence, portion, homologue, variant or derivative thereof, in an amount sufficient to prevent, stimulate, induce, promote, increase, immunize against, or enhance an immune response against MTB in the subject. In one aspect, the immune response provides the subject with protection against MTB infection or pathology, or one or more physiological conditions, disorders, illnesses, diseases or symptoms caused by or associated with MTB infection or pathology.

In another embodiment, the present invention includes a method of treating, preventing, or immunizing a subject against MTB infection, comprising administering to a subject an amount of a protein or peptide comprising, consisting of, or consisting essentially of an amino acid sequence of a Mycobacterium protein or peptide, or a variant, homologue, derivative or subsequence thereof, wherein the protein or peptide comprises at least two amino acid sequences selected from any one of Tables 1-5 (SEQ ID NOS:1-718) or a subsequence, portion, homologue, variant or derivative thereof, in an amount sufficient to treat, prevent, or immunize the subject for MTB infection, wherein the protein or peptide comprises or consists of a Mycobacterium T cell epitope that elicits, stimulates, induces, promotes, increases, or enhances an anti-MTB T cell immune response. In one aspect, the one or more amino acid sequences are selected from any one of those sequences set forth in Tables 1-5 (SEQ ID NOS:1-718), or a subsequence, portion, homologue, variant or derivative thereof; a fusion protein comprising one or more amino acid sequences selected from any one of those sequences set forth in Tables 1-5 (SEQ ID NOS:1-718); or a pool of 2 or more peptides selected from the amino acid sequences set forth in those sequences set forth in any one of Tables 1-5 (SEQ ID NOS:1-718). In one aspect, the anti-MTB T cell response is a CD8+, a CD4+ T cell response, or both. In another aspect, the T cell epitope is conserved across two or more clinical isolates of MTB or two or more circulating forms of MTB. In another aspect, the MTB infection is an acute infection. In another aspect, the subject is a mammal or a human. In another aspect, the method reduces MTB bacterial titer, increases or stimulates MTB bacterial clearance, reduces or inhibits MTB bacterial proliferation, reduces or inhibits increases in MTB bacterial titer or MTB bacterial proliferation, reduces the amount of a MTB bacterial protein or the amount of a MTB bacterial nucleic acid, or reduces or inhibits synthesis of a MTB bacterial protein or a MTB bacterial nucleic acid. In another aspect, the method reduces one or more adverse physiological conditions, disorders, illness, diseases, symptoms or complications caused by or associated with MTB infection or pathology. In another aspect, the method improves one or more adverse physiological conditions, disorders, illness, diseases, symptoms or complications caused by or associated with MTB infection or pathology. In another aspect, the symptom is fever or chills, cough, shortness of breath or difficulty breathing, fatigue, muscle or body aches, headache, new loss of taste or smell, sore throat, congestion or runny nose, nausea or vomiting, or diarrhea. In another aspect, the method reduces or inhibits susceptibility to MTB infection or pathology. In another aspect, the protein or peptide, or a subsequence, portion, homologue, variant or derivative thereof, is administered prior to, substantially contemporaneously with or following exposure to or infection of the subject with MTB. In another aspect, a plurality of MTB T cell epitopes are administered prior to, substantially contemporaneously with or following exposure to or infection of the subject with MTB. In another aspect, the protein or peptide, or a subsequence, portion, homologue, variant or derivative thereof is administered within 2-72 hours, 2-48 hours, 4-24 hours, 4-18 hours, or 6-12 hours after a symptom of MTB infection or exposure develops. In another aspect, the protein or peptide, or a subsequence, portion, homologue, variant or derivative thereof is administered prior to exposure to or infection of the subject with MTB. In another aspect, the method further comprises administering a modulator of immune response prior to, substantially contemporaneously with or following the administration to the subject of an amount of a protein or peptide. In another aspect, the modulator of immune response is a modulator of the innate immune response. In another aspect, the modulator is IL-6, IFN-γ, TGF-3, or IL-10, or an agonist or antagonist thereof.

In another embodiment, the present invention includes a method of treating, preventing, or immunizing a subject against MTB infection, comprising administering to a subject the composition of one or more proteins, peptides or multimers in an amount sufficient to treat, prevent, or immunize the subject for MTB infection. In one aspect, the MTB infection is an acute infection. In another aspect, the method reduces MTB bacterial titer, increases or stimulates MTB bacterial clearance, reduces or inhibits MTB bacterial proliferation, reduces or inhibits increases in MTB bacterial titer or MTB bacterial proliferation, reduces the amount of a MTB bacterial protein or the amount of a MTB bacterial nucleic acid, or reduces or inhibits synthesis of a MTB bacterial protein or a MTB bacterial nucleic acid. In another aspect, the method reduces one or more adverse physiological conditions, disorders, illness, diseases, symptoms or complications caused by or associated with MTB infection or pathology. In another aspect, the method improves one or more adverse physiological conditions, disorders, illness, diseases, symptoms or complications caused by or associated with MTB infection or pathology. In another aspect, the symptom is fever or chills, night sweats, cough, loss of appetite, weight loss, fatigue, nail clubbing, chest pain, prolonged cough, sputum production, coughing blood, scarring in the lungs, bleeding or erosion of the pulmonary artery or Rasmussen's aneurysm, shortness of breath or difficulty breathing, fatigue, muscle or body aches, headache, new loss of taste or smell, sore throat, congestion or runny nose, nausea, vomiting, or diarrhea. In another aspect, the method reduces or inhibits susceptibility to MTB infection or pathology. In another aspect, the composition is administered prior to, substantially contemporaneously with or following exposure to or infection of the subject with MTB. In another aspect, the composition is administered prior to, substantially contemporaneously with or following exposure to or infection of the subject with MTB. In another aspect, the composition is administered within 2-72 hours, 2-48 hours, 4-24 hours, 4-18 hours, or 6-12 hours after a symptom of MTB infection or exposure develops. In another aspect, the composition is administered prior to exposure to or infection of the subject with MTB.

In another embodiment, the present invention includes a peptide or peptides that are immunoprevalent or immunodominant in a bacteria obtained by a method consisting of, or consisting essentially of: obtaining an amino acid sequence of the bacteria; determining one or more sets of overlapping peptides spanning one or more bacteria antigen using unbiased selection; synthesizing one or more pools of bacteria peptides comprising the one or more sets of overlapping peptides; combining the one or more pools of bacteria peptides with Class I major histocompatibility proteins (MHC), Class II MHC, or both Class I and Class II MHC to form peptide-MHC complexes; contacting the peptide-MHC complexes with T cells from subjects exposed to the bacteria; determining which pools triggered cytokine release by the T cells; and deconvoluting from the pool of peptides that elicited cytokine release by the T cells, which peptide or peptides are immunoprevalent or immunodominant in the pool. In one aspect, the bacteria is a Mycobacterium. In another aspect, the Mycobacterium is MTB. In another aspect, the immunodominant peptides are selected from 1, 2 or more peptides selected from the amino acid sequences set forth in any one of Tables 1-5 (SEQ ID NOS:1-718). In another aspect, the immunodominant peptides are selected from 1, 2 or more peptides selected from the amino acid sequences set forth in those sequences set forth in any one of Tables 1-5 (SEQ ID NOS:1-718).

In another embodiment, the present invention includes a method of selecting an immunoprevalent or immunodominant peptide or protein of a bacteria comprising, consisting of, or consisting essentially of: obtaining an amino acid sequence of the bacteria; determining one or more sets of overlapping peptides spanning one or more bacteria antigen using unbiased selection; synthesizing one or more pools of bacteria peptides comprising the one or more sets of overlapping peptides; combining the one or more pools of bacteria peptides with Class I major histocompatibility proteins (MHC), Class II MHC, or both Class I and Class II MHC to form peptide-MHC complexes; contacting the peptide-MHC complexes with T cells from subjects exposed to the bacteria; determining which pools triggered cytokine release by the T cells; and deconvoluting from the pool of peptides that elicited cytokine release by the T cells, which peptide or peptides are immunoprevalent or immunodominant in the pool. In one aspect, the bacteria is a Mycobacterium. In another aspect, the Mycobacterium is MTB. In another aspect, the immunodominant peptides are selected from 1, 2 or more peptides selected from the amino acid sequences set forth in any one of Tables 1-5 (SEQ ID NOS:1-718). In another aspect, the immunodominant peptides are selected from 1, 2 or more peptides selected from the amino acid sequences set forth in those sequences set forth in any one of Tables 1-5 (SEQ ID NOS:1-718).

In another embodiment, the present invention includes a polynucleotide that expresses one or more peptides or proteins, comprising, consisting of, or consisting essentially of an amino acid sequence selected from any one of those sequences set forth in Tables 1-5 (SEQ ID NOS:1-718), or a subsequence, portion, homologue, variant or derivative thereof; a fusion protein comprising one or more amino acid sequences selected from any one of those sequences set forth in Tables 1-5 (SEQ ID NOS:1-718); or a pool of 2 or more or more peptides comprising, consisting of, or consisting essentially of amino acid sequences selected from any one of those sequences set forth in Tables 1-5 (SEQ ID NOS:1-718). In one aspect, the vector comprises the polynucleotide of claim that expresses one or more peptides or proteins, comprising, consisting of, or consisting essentially of an amino acid sequence selected from any one of those sequences set forth in Tables 1-5 (SEQ ID NOS:1-718), or a subsequence, portion, homologue, variant or derivative thereof; a fusion protein comprising one or more amino acid sequences selected from any one of those sequences set forth in Tables 1-5 (SEQ ID NOS:1-718); or a pool of 2 or more or more peptides comprising, consisting of, or consisting essentially of amino acid sequences selected from any one of those sequences set forth in Tables 1-5 (SEQ ID NOS:1-718), a bacterial vector, or a host cell the comprises the same.

In another embodiment, the present invention includes a polynucleotide that expresses one or more peptides or proteins comprising, consisting of, or consisting essentially of an amino acid sequence selected from any one of those sequences set forth in Tables 1-5 (SEQ ID NOS:1-718), or a subsequence, portion, homologue, variant or derivative thereof; a fusion protein comprising one or more amino acid sequences selected from any one of those sequences set forth in Tables 1-5 (SEQ ID NOS:1-718); or a pool of 2 or more peptides selected from any one of those sequences set forth in Tables 1-5 (SEQ ID NOS:1-718). In one aspect, the vector comprises the polynucleotide of claim that expresses one or more peptides or proteins comprising, consisting of, or consisting essentially of an amino acid sequence selected from any one of those sequences set forth in Tables 1-5 (SEQ ID NOS:1-718), or a subsequence, portion, homologue, variant or derivative thereof; a fusion protein comprising one or more amino acid sequences selected from any one of those sequences set forth in Tables 1-5 (SEQ ID NOS:1-718); or a pool of 2 or more peptides selected from any one of those sequences set forth in Tables 1-5 (SEQ ID NOS:1-718), a bacterial vector, or a host cell that comprises the same.

### BRIEF DESCRIPTION OF THE DRAWINGS

FIGS. 1A to 1E provide an example of infection-stage specific epitope pools tested in IGRA−, IGRA+ and ATB individuals. FIG. 1A—MTB300, FIG. 1B—ATB-specific, FIG. 1C—LTBI-specific, FIG. 1D—MTB300 with the addition of ATB-specific, and FIG. 1E—LTBI/ATB shared epitope pool.

FIGS. 2A to 2C provide a non-limiting example of the breadth and dominance of epitopes in mid-treatment ATB participants. (FIG. 2A) Number of epitopes recognized by each participant. Each dot is one participant, n=21; median±interquartile range is shown. (FIG. 2B) Distribution of recognized epitopes by the number of participants recognizing each epitope. (FIG. 2C) Epitopes ranked based on the magnitude of response (solid line—% of total response, dotted line—total SFC). Black dotted lines indicate the top 55 epitopes.

FIGS. 3A to 3E provide a non-limiting example of immunodominant antigens in ATB. (FIG. 3A) Number of ORFs corresponding to recognized epitopes by each participant. Each dot represents one participant, n=21, median±interquartile range is shown. (FIG. 3B) Distribution of recognized ORFs per protein category. (FIG. 3C). The identified antigens (black bars) were divided into protein categories (Mycobrowser) and compared to the H37Rv genome (grey bars). Chi-square test. (FIG. 3D) Antigenic islands identified by a 5-gene window spanning the H37Rv genome. ATB (black) compared to healthy IGRA+ (orange, (22)). (FIG. 3E) Proteins within each antigenic island, % of total response per antigen across the proteome-wide screen. ATB (black bars) and healthy IGRA+ (orange bars).

FIGS. 4A and 4B provide a non-limiting example of the hierarchy in T cell reactivity against TB vaccine and IGRA antigens. Magnitude of response, expressed as the total magnitude of response (black bars, left y-axis) or frequency of participants responding (grey bars, right y-axis), amongst the participants. (FIG. 4A) ATB, n=21. (FIG. 4B) Healthy IGRA+, n=63 (37), for comparison purposes. Rv number for each antigen are indicated on the x-axis.

FIGS. 5A to 5G provide a non-limiting example of T cell responses specific for different protein categories. (FIG. 5A) Frequency of cytokine-producing, IFNγ, TNFα, and IL-2, CD4 T cells in response to PC85, PC71, and MTB300. (FIG. 5B) Frequency of cytokine-producing, IFNγ, TNFα, and IL-2, CD8+ T cells in response to PC85, PC71, and MTB300. (FIG. 5C) Percentage pool-specific IFNγ, TNFα, and IL-2 production by CD4 T cells expressing each of the seven possible combinations. (FIG. 5D) Pie charts representing single, dual and triple cytokine producing CD4 T cells. Each section of the pie chart represents a specific combination of cytokines, as indicated by the color. (FIG. 5E-FIG. 5G) Proportion of CCR7+CD45RA− (central memory), CCR7−CD45RA− (effector memory), CCR7+CD45RA+ (naïve), and CCR7−CD45RA+ (T_{EMRA}) T cells for each peptide pool. (FIG. 5E) IFNγ, (FIG. 5F) IL-2, (FIG. 5G) TNFα. (A-C, E-G) Each point represents one participant, n=10, median±interquartile range is shown. Two-tailed Mann-Whitney test.

FIGS. 6A to 6C provide a non-limiting example of cytokine response against the ATB-specific peptide pool, ATB116. (FIG. 6A) Magnitude of response (total SFC for IFNγ) against ATB116 and MTB300 in ATB (at diagnosis; n=24), IGRA+ (n=25), and IGRA− (n=43) from Sri Lanka. (FIG. 6B) Frequency of cytokine-producing, IFNγ, TNFα, and IL-2, CD4 T cells in response to ATB116 and MTB300 in ATB (at diagnosis, n=9) and IGRA− (n=9) participants. (FIG. 6C) Magnitude of response (total SFC for IFNγ) against ATB116 and MTB300 in ATB (at diagnosis; n=9), ATB (mid-treatment; n=12), IGRA+ (n=20), and IGRA− (n=10) household contacts from Moldova. (FIG. 6A-FIG. 6C) Each dot represents one participant, median±interquartile range is shown. Two-tailed Mann Whitney test. (FIG. 6A, FIG. 6C) The dashed line indicates the cut-off used for a positive response (50 SFC/10⁶ PBMCs).

FIGS. 7A to 7C provide anon-limiting example of the characterization of ATB116- and MTB300-specific CD4 T cell responses. FIG. 7A-FIG. 7C) Frequency of cytokine-producing, IFNγ (FIG. 7A), IL-2 (FIG. 7B), and TNFα (FIG. 7C), CD4 T cells in response to ATB116 and MTB300 in longitudinal samples from ATB patients (visit 1; at diagnosis, visit 2; mid-treatment, visit 3; end of treatment, n=7). Each point represents one participant, median±interquartile range is shown. Wilcoxon signed rank test.

FIGS. 8A to 8D show the diagnostic use of ATB116. Receiver Operating Characteristic (ROC) curves for Sri Lanka (FIG. 8A, FIG. 8B) and Moldova (FIG. 8C, FIG. 8D). ATB vs. IGRA+ (FIG. 8A, FIG. 8C) and ATB vs. IGRA− (FIG. 8B, FIG. 8D). The x-axis shows False positive rate (100%-specificity %) and y-axis shows true positive rate (sensitivity %). Each point on the curve represents a different threshold value used to calculate the true positive rate and false positive rate. Area under curve (AUC) is shown for predictive performance.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention. Numerous variations, changes, and substitutions will now occur to those skilled in the art without departing from the invention. It should be understood that various alternatives to the embodiments of the invention described herein may be employed in practicing the invention. To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims. Unless specifically stated or obvious from context, as used herein, the term “or” is understood to be inclusive.

### Example 1

The inventors have identified T cell epitopes which are exclusively recognized by individuals with active tuberculosis (TB) infection and not in those that are TB neg or those with latent TB infection.

First, the inventors compiled a peptide library of 21,220 candidate T cell epitopes. The peptide library includes a library of 20,610 Mtb-derived HLA class II predicted binding peptides that was previously screened in individuals with latent TB infection and which resulted in the commonly used peptide megapool “MTB300” that the inventors previously generated. The 20,610 peptides represent every ORF, with 2-10 peptides per ORF, with an average of 5 that are present in the Mtb genome. The remaining peptides in the screened library also include 1,660 variants that were not totally conserved in the selected Mtb genomes (5 complete genomes CDC1551, F11, H37Ra, H37Rv, and KZN1435) and 16 draft assemblies. The remaining 610 peptides include 93 peptides that are not found in Mtb but present in the Mycobacterium bovis BCG strains Mexico, Tokyo 172, and Pasteur 1173P2, and 517 peptides that are 15-mers overlapping by 10 amino acids spanning the entire sequence of 12 TB vaccine candidate and Interferon Gamma Release Assay (IGRA; a diagnostic test for TB) antigens. The vaccine candidates include ID93:GLA-SE (Rv3619c, Rv3620c, Rv1813, and Rv2608), H1:IC31 (ESAT-6 and Ag85B), H4:IC31 (Ag85B, TB10.4), H56:IC31 (Ag85B, ESAT-6 and Rv2660c), M72/AS01E (Mtb32A, PPE18), and three candidates with Ag85A alone (Ad5 Ag85A, ChAdOx1-85A/MVA85A, and MVA85A). The IGRA antigens include ESAT-6, which is also a vaccine candidate antigen, and CFP10.

The peptide library was arranged in pools of ˜20 peptides per pool and tested for reactivity in individuals who were 3-4 m post diagnosis (mid-treatment) of active TB infection using IFNg Fluorospot assays. Any peptide pool that were recognized by 2 or more subjects, along with the 10 pools with the highest magnitude of responses per subject was deconvoluted. The cohort of subjects with active TB undergoing treatment recognized an average of 10 individual epitopes (range 1-38). This translates to an average of 8 ORFs recognized (range 1-18). In total the inventors detected reactivity against 178 individual epitopes and 36 of those (20%) were recognized by 2 or more subjects. These 178 epitopes correspond to 105 ORFs, which are predominantly found in the cell wall and cell processes category of the functional categories that define the ORFs in Mtb, followed by conserved hypotheticals, PE/PPE, and intermediary metabolism and respiration.

To define Mtb-infection stage specific epitopes the inventor used MTB300, defined from the proteome-wide screen in individuals with LTBI, and the 178 epitopes identified in the present screen. The inventors divided these peptides into disease-specific categories and identified 113 epitopes (Table 1) which are only recognized by individuals with active TB (ATB-specific), 137 peptides (Table 2) that are LTBI-specific and 65 peptides (Table 3) that are shared between ATB and LTBI.

### Example 2

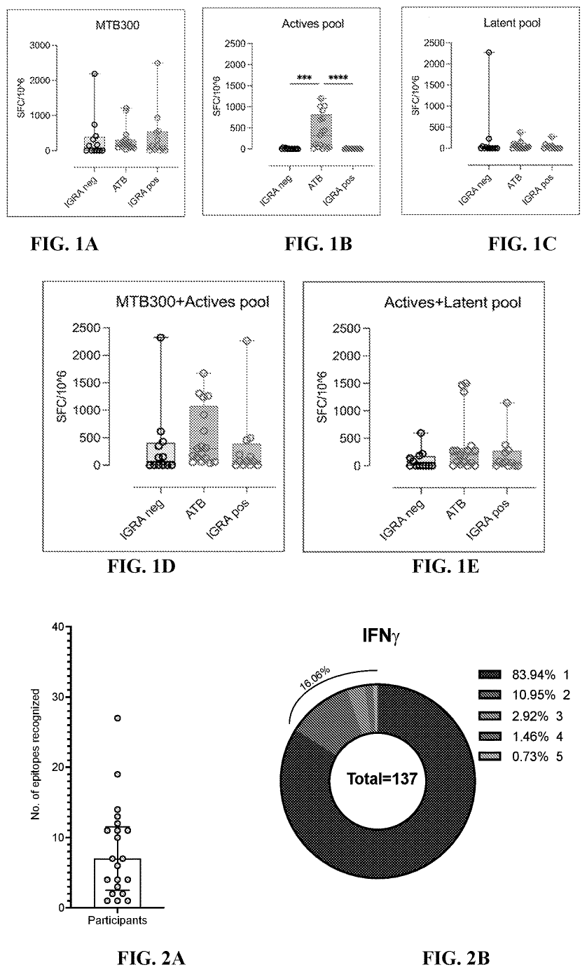

The inventors measured IFNg responses against these pools using Fluorospot assays in a cohort of individuals with active TB (recruited at diagnosis) from Sri Lanka and South Africa. The inventors compare reactivity to that observed in IGRA+ (i.e. LTBI) and IGRA− individuals. The ATB-specific peptide pool shows the most promising results with exclusive reactivity in individuals with ATB (FIGS. 1A to 1E).

### Example 3

The inventors performed a proteome-wide screen of 20,610 Mtb derived peptides in 21 Active TB (ATB) patients 3-4 months post-diagnosis of pulmonary TB (mid-treatment) using an IFN-γ and IL-17 Fluorospot assay. Responses were mediated exclusively by IFN-γ, and identified a total of 137 unique epitopes, with each patient recognizing, on average, 8 individual epitopes and 22 epitopes (16%) recognized by 2 or more participants. Responses were predominantly directed against antigens part of the cell wall and cell processes category. Testing of 517 peptides spanning TB vaccine candidate and ESAT-6 and CFP10 antigens also revealed differential recognition between ATB participants mid-treatment and healthy IGRA+ participants of several vaccine antigens. An ATB-specific peptide pool consisting of epitopes exclusively recognized by participants mid-treatment, allowed distinguishing participants with active pulmonary TB from healthy interferon gamma release assay (IGRA)+/−participants from diverse geographical locations. Analysis of longitudinal samples indicated decreased reactivity during treatment for pulmonary TB. Together, these results show that a proteome-wide screen of T cell reactivity identifies epitopes and antigens that are differentially recognized depending on the Mtb infection stage, and such epitopes are disclosed herein. In the various methods and compositions described and embodied herein, these epitopes are useful diagnostics and therapeutics, including but not limited to vaccine candidates and measuring correlates of protection.

### Example 4: Breadth of Responses to a Comprehensive Library of Predicted HLA Class II Epitopes in Participants with ATB

The inventors previously used a proteome-wide peptide library of 20,610 Mtb-derived 15-mer peptides predicted promiscuous HLA class II binders to determine the repertoire of T cell antigens and epitopes recognized in ATB.

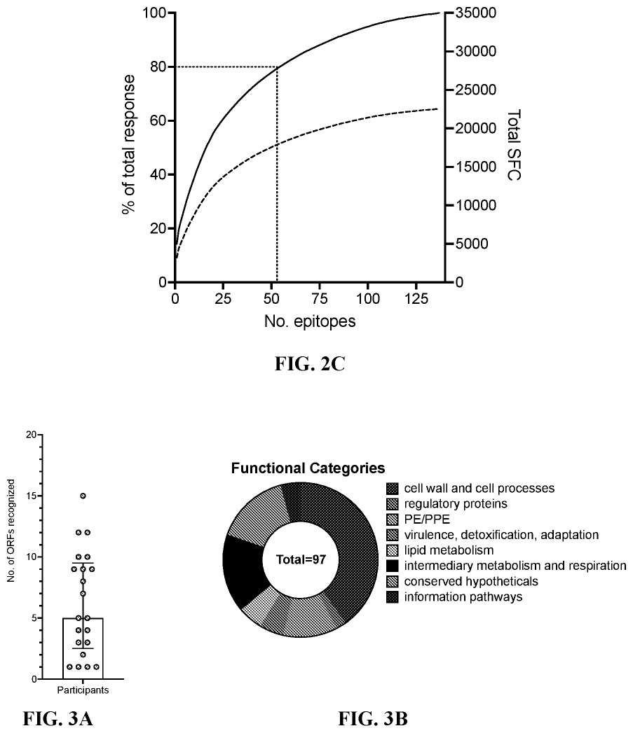

The proteome-wide peptide library was arranged into 1036 peptide pools of 20 peptides each. T cell reactivity against the 1036 peptide pools was measured by ex vivo production of IFNγ and IL-17 using Fluorospot assays and PBMCs from 21 participants mid-treatment for their ATB infection (3-4 months post diagnosis) recruited from the Universidad Peruana Cayetano Heredia site (Peru). A total of 78 unique peptide pools were selected for deconvolution, corresponding to the ten peptide pools with the highest response magnitude per participant, and/or that were recognized by at least two different participants. A total of 137 individual epitopes were identified (Table 5). Each participant recognized an average of 8 unique epitopes (range 1-27, median 7), underlining the breadth of responses to Mtb (FIG. 2A). Among the 137 individual epitopes, 22 (16%) were recognized by multiple participants (FIG. 2B). When epitopes were ranked based on magnitude the top 55 epitopes accounted for 80% of the total response (FIG. 2C). In conclusion, the breadth of responses detected in ATB is broad, although narrower (p=0.002, two-tailed unpaired Student's t-test) compared to the previous results in healthy IGRA+ participants who recognized on average 24 epitopes (22).

### Example 5: Immunodominant Antigens in Cellular Responses in ATB Participants

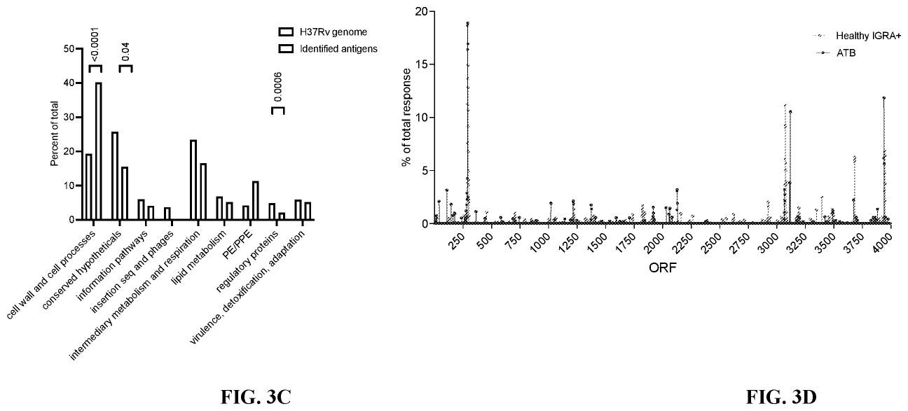

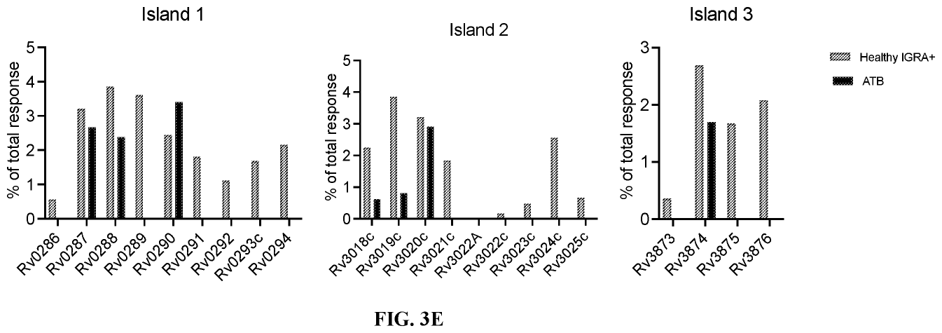

The epitopes were mapped to individual Mtb ORFs using H37Rv as a reference genome. A total of 97 ORFs were recognized, with each participant recognizing, on average, 6 ORFs (median 5, FIG. 3A). As expected, the well-known antigens Rv0288 (TB10.4), Rv3875 (ESAT-6), Rv3874 (CFP10), and Rv3615c (included in the “ESAT-6 free” IGRA test (35) were the most frequently recognized. However, nine novel antigens, which have not been previously described as antigens for Mtb, were also identified (Table 5).

Using the Mycobrowser tool (36), the inventors next determined the protein categories to which the identified antigenic ORFs belonged. As previously observed for IGRA+ participants (37), essentially every protein category was represented (FIG. 3B). However, the ORFs antigenic in ATB were predominantly found in the cell wall and cell processes categories, followed by conserved hypotheticals, PE/PPE, and intermediary metabolism and respiration categories (FIG. 3B). Compared to the H37Rv genome, the antigenic ORFs were overrepresented in the cell wall, cell processes category, and the PE/PPE categories and underrepresented in conserved hypotheticals (FIG. 3C). The overrepresentation of the cell wall and cell processes category appears to be specific for the ATB cohort, because these antigen categories were not overrepresented in the previous screen of IGRA+ participants (22).

Reactivity in healthy IGRA+ participants (22) previously identified three “antigenic islands” of clustered antigen genes that comprised secreted and non-secreted Mtb proteins involved in type 7 secretion systems. Indeed, all three antigenic islands were also identified in the present screen of ATB participants. (FIG. 3D). In contrast to IGRA+ participants in the previous screen, the breadth of responses to antigens targeting the antigenic islands was narrower in the participants with ATB (FIG. 3E). For example, for island 1, only 3 antigens represented in the predicted peptide library of 20,610 peptides were recognized by participants with ATB, compared to 9 recognized by healthy IGRA+ participants. The same is true for antigens in islands 2 and 3. In conclusion, the data suggest that different patterns of antigenic ORFs might be associated with ATB vs. healthy IGRA+ participants.

TABLE 1
ATB-specific

| SEQ | | from |
|---|---|---|
| ID | | ORF |
| NO: | Sequence | prediction |
| 1 | ILLLVAVVALLFTSR | Rv1888c |
| 2 | EATGLLVALRALADI | Rv2095c |
| 3 | ACLLGLTILLLAVNR | Rv0176 |
| 4 | AEMKTDAATLAQEAG | Rv3874 |
| 5 | AIFMGCYLRFLRPGR | Rv3063 |
| 6 | AQHRHRYLTMVNVGR | Rv1351 |
| 7 | GWLVLIAVLALSLVR | Rv3635 |
| 8 | ILVGAAAAVVLVAMR | Rv2051c |
| 9 | IRRLTAAVALAAAGA | Rv0176 |
| 10 | IVVVAVLALGRRIPP | Rv1508c |
| 11 | LIGFALLAFCSVVAR | Rv1349 |
| 12 | TVIILFLAGAVVNLK | Rv3645 |
| 13 | AAAAAYEAAFAATVP | Rv1807 |
| 14 | AAEVLKILLGHGRVY | Rv2338c |
| 15 | AAIHDQFVATLASSA | Rv1195 |
| 16 | AAIQARAAATAFEAA | Rv3125c |
| 17 | AAVLVLFTLIFFLYG | Rv0205 |
| 18 | ADADIIMLAPSNPVV | Rv3261 |
| 19 | ADAQYPASTAFLATT | Rv0144 |
| 20 | ADGLRADPRNQRVLL | Rv0016c |
| 21 | AGAQAEGAAAAYEAA | Rv1807 |
| 22 | AGGIYGDFFNFYLCD | Rv0590A |
| 23 | AGHTHYLIIDDVDQV | Rv0284 |
| 24 | AILLVLTVLQLRITH | Rv2040c |
| 25 | ALCLIFIIMLIITRS | Rv0676c |
| 26 | ALCLIFIIMLITTRS | Rv0676c |
| 27 | ANRWIILGVSAQAIA | Rv1997 |
| 28 | APVMWALSASLGWIL | Rv1227c |
| 29 | AQEWLRFGWFFLATR | Rv3782 |
| 30 | AQEWLRFGWFFLVTR | Rv3782 |
| 31 | ARFFGRNVNVPLMII | Rv3071 |
| 32 | ARILLLVPSISLLSQ | Rv2024c |
| 33 | ASGPKVVIDGKDQNV | Rv3763 |
| 34 | ATRFRPIIRLTVEWL | Rv3887c |
| 35 | ATRVRPIIRLTVEWL | Rv3887c |
| 36 | AVGFTPEQLEIELNW | Rv1894c |
| 37 | AVLRRLEWHFTLVFA | Rv3436c |
| 38 | DIKVQFQSGGNNSPA | Rv1886c |
| 39 | DLVWDFRLPRVVSIN | Rv3432c |
| 40 | DQLVCRVVVPAVATT | Rv0107c |
| 41 | DQLVRRVVVPAVATT | Rv0107c |
| 42 | DRYINWAKDQPQYPY | Rv3784 |
| 43 | DSQKLLAWQTTNASM | Rv0040c |
| 44 | EATGLLVALRALANI | Rv2095c |
| 45 | EDALRLLSLPRVVGV | Rv3646c |
| 46 | EDAVRNAKAAVEEGI | Rv0440 |
| 47 | EDLVRAYHAMSRTHE | Rv0288 |
| 48 | EDQVRQAIQSLDIAI | Rv0361 |
| 49 | ELWVGFTAVSALLIL | Rv0876c |
| 50 | EQDLRYLRGLGQFSD | Rv0573c |
| 51 | ERLVNLVIALLSTRG | Rv2096c |
| 52 | ESLRLYDSSYHAELF | Rv2032 |
| 53 | ESPVSYHAFDPELQL | Rv0276 |
| 54 | EVFLVIDNLYGFGRD | Rv0284 |
| 55 | FCLFGVLFVLLRRGR | Rv3794 |
| 56 | FFPFVLLLAATALYR | Rv0284 |
| 57 | FGEVIYVSAELKQKH | Rv0731c |
| 58 | FNLMLWDLDRRMRRG | Rv0276 |
| 59 | FPIFAVTHCRDVVVA | Rv1894c |
| 60 | FRAFYALPAENFKTR | Rv1629 |
| 61 | GAAAAYEAAFAATVP | Rv1807 |
| 62 | GAMIRAQAGLLEAEH | Rv2346c |
| 63 | GGLLARFPGFYIPFL | Rv0160c |
| 64 | GIDEISDNASVLVSV | Rv3493c |
| 65 | GIGRPARQRTTTYAL | Rv1173 |
| 66 | GILIVGMIVALVATG | Rv0284 |
| 67 | GITVADNVAAFSELG | Rv0694 |
| 68 | GLSSVFMAILNTRNM | Rv3910 |
| 69 | GLSSVFMAILNTRNV | Rv3910 |
| 70 | GLTAILMLYSIVIIR | Rv0017c |
| 71 | GNGYRLTGKLHIRGK | Rv1890c |
| 72 | GPILLYVLLPLLDLR | Rv3252c |
| 73 | GSTADFTGTTLNSLR | Rv3818 |
| 74 | GSWLLDLTAIALRES | Rv1122 |
| 75 | ILLLVAVVALLFTGR | Rv1888c |
| 76 | ILLRDALHATAVRIL | Rv3383c |
| 77 | IRLALSLGFRVRVAA | Rv1681 |
| 78 | IRRLLPALVVVLAGC | Rv1565c |
| 79 | ISAERYESLMEELED | Rv0268c |
| 80 | ISPQTLFFPFVLLLA | Rv0284 |
| 81 | IVDYYNFFLSGGFLA | Rv3796 |
| 82 | KAWHQRTPARAEQVA | Rv1813c |
| 83 | KDDIFYYVYGLLHDP | Rv2024c |
| 84 | KTPEWAAALSGLAAG | Rv1442 |
| 85 | LGTAAGVLLIGLVRW | Rv1111c |
| 86 | LHRAFAYTSNIFIRD | Rv3796 |
| 87 | LIIDDVDQVPDSPAM | Rv0284 |
| 88 | LILFAIVSVVAIVVL | Rv0174 |
| 89 | LPFVILIGLIVSRQW | Rv0585c |
| 90 | LPLRRLLGLVAAGLD | Rv0290 |
| 91 | LPTVRPIVAAVAERG | Rv1599 |
| 92 | LRRNSANLLVLAGAQ | Rv3365c |
| 93 | LSRAGLFFVPLAVAA | Rv0267 |
| 94 | LVYRFIRDTTWVSVR | Rv3557c |
| 95 | MLMPCFAQLYDELGI | Rv3522 |
| 96 | NRGYVLSQPGLRKLL | Rv3782 |
| 97 | NVPSYRVSQYDIVDV | Rv3458c |
| 98 | NVRDQVFNLFEVLGV | Rv0244c |
| 99 | PDWVNTIFLSTRFRA | Rv3818 |
| 100 | QLYQYRFTTVAELRR | Rv0235c |
| 101 | QSGFIAAAVLLSVLG | Rv0290 |
| 102 | RATFRALGSTGHRFL | Rv0836c |
| 103 | RILFAFDPARQAILL | Rv3182 |
| 104 | SMLFLPVRILTSPIT | Rv3125c |
| 105 | SPPGLPVAAVAEQAP | Rv2095c |
| 106 | SSAGLMVAAASPYVA | Rv1196 |
| 107 | SSWRPVLLIPLTFAL | Rv3635 |
| 108 | TATATLLPFEEAPEM | Rv1196 |
| 109 | TTPSVLFLLLKTIAT | Rv1005c |
| 110 | VLMMELNRISSHLVA | Rv3148 |
| 111 | WPLLIFWLSYTGHRH | Rv1565c |
| 112 | YLRIAVANMANAVKK | Rv0266c |
| 113 | YLYPIVALRIRGIAL | Rv0276 |

TABLE 2
LTBI-specific

| SEQ | | |
|---|---|---|
| ID | | ORF from |
| NO: | Sequence | prediction |
| 114 | DRWLDLRYVGPASAD | Rv0289 |
| 115 | LENDNQLLYNYPGAL | Rv3330 |
| 116 | AAVVRFQEAANKQKQ | Rv3874 |
| 117 | QQIKFAALSARAVAL | Rv3024c |
| 118 | ADYLRMWIQAATVMS | Rv2123 |
| 119 | EQQWNFAGIEAAASA | Rv3875 |
| 120 | GEEYLILSARDVLAV | Rv3418c |
| 121 | AAGVAAWSLIALMIP | Rv0290 |
| 122 | AALPLLFFALAGQRI | Rv2874 |
| 123 | AGCQTYKWETFLTSE | Rv3804c |
| 124 | ANATVYMIDSVLMPP | Rv2875 |
| 125 | DYVRMWVQAATVMSA | Rv3018c |
| 126 | ELFVAAYVPYVAWLV | Rv3022c |
| 127 | GQNYTYKWETFLTRE | Rv0129c |
| 128 | RQSGATIADVLAEKE | Rv3876 |
| 129 | YRIAARPGAVTRRAA | Rv3015c |
| 130 | AAGAQLLWQLPLLSI | Rv0290 |
| 131 | AAIHEMFVNTLVASS | Rv1791 |
| 132 | AAVPAVGAAAGAPAA | Rv3021c |
| 133 | AGWLAFFRDLVARGL | Rv3115 |
| 134 | AHGETVSAVAELIGD | Rv3024c |
| 135 | ALSVLVGLTAATVAI | Rv0291 |
| 136 | AVDGRFAVPQILGDE | Rv3012c |
| 137 | EIGWEAGTAAPDEIP | Rv0292 |
| 138 | EIVQFLEETFAAYDQ | Rv3018c |
| 139 | GNGVVALRNAQLVTF | Rv0289 |
| 140 | GTVVLTATFALGAAL | Rv2874 |
| 141 | LNYRPLLPKDRRMII | Rv0293c |
| 142 | LRGLLSTFIAALMGA | Rv3115 |
| 143 | MHVSFVMAYPEMLAA | Rv1195 |
| 144 | RVPEDLLAMVVAVEQ | Rv0299 |
| 145 | YLGLEVLTRARAALT | Rv3115 |
| 146 | YVAWMSATAALAREA | Rv1802 |
| 147 | AARLLSIRAMSTKFS | Rv0291 |
| 148 | AKLMRDIPFRVGAVV | Rv0294 |
| 149 | ALTALIRDPPADSTG | Rv0292 |
| 150 | AQLSQLISLLPSTLQ | Rv1808 |
| 151 | ARMWIQAATTMASYQ | Rv0256c |
| 152 | DYVRMWVQAATAMSA | Rv3018c |
| 153 | EVVDYLGIPASARPV | Rv0289 |
| 154 | GADATAAAAFEQFLA | Rv2024c |
| 155 | GAMVATNFFGINTIP | Rv0453 |
| 156 | GWIISNIFGAIPVLG | Rv3021c |
| 157 | IDLNVLLSAAINFFL | Rv0985c |
| 158 | ILPIAEMSVVAMEFG | Rv3025c |
| 159 | LAAAAAWDALAAELY | Rv1808 |
| 160 | LALVGFLGGLITGIS | Rv2874 |
| 161 | LALVGFLGGLITGTS | Rv2874 |
| 162 | LQSLWANFYELLADA | Rv1366 |
| 163 | LSPISNMVSMANNHM | Rv1196 |
| 164 | MWDPDVYLAFSGHRN | Rv0294 |
| 165 | MYRELLELVAADVES | Rv0690c |
| 166 | QASPDLLRGLLSTFI | Rv3115 |
| 167 | RCALHWFPGSHLLAC | Rv0293c |
| 168 | VTVDAAVLAAIDADA | Rv0298 |
| 169 | AAIHEMFVNTLQMSS | Rv1788 |
| 170 | AALPAVGAAAGAPAA | Rv3021c |
| 171 | AEHQAIVRDVLAAGD | Rv1198 |
| 172 | AEHQAIVRDVLAASD | Rv1198 |
| 173 | AEKFKEDVINDFVSS | Rv3024c |
| 174 | AELMILIATNLLGQN | Rv1361c |
| 175 | ALLPRAGAAAAAALP | Rv1172c |
| 176 | ALQSHDDVALVSVMW | Rv3025c |
| 177 | ALSAEYAAVAQELSV | Rv3022c |
| 178 | ALSRVHSMFLGTGGS | Rv1172c |
| 179 | AQIYQAVSAQAAAIH | Rv1788 |
| 180 | ASIIRLVGAVLAEQH | Rv3115 |
| 181 | AVHVWLRLPAGRVEI | Rv1366 |
| 182 | AVPLRLLGGLHRMVL | Rv0690c |
| 183 | AYAQRVYQANRAAGS | Rv0298 |
| 184 | DESWQQFRQELIPLL | Rv0294 |
| 185 | FGQNTASIAATEAQY | Rv1705c |
| 186 | FGQNTGAIAAAEARY | Rv1802 |
| 187 | FGQNTSAIAAAEAQY | Rv1789 |
| 188 | FVQALTTAAASYASV | Rv2853 |
| 189 | GWIISNIFGAIPVLA | Rv3021c |
| 190 | IGLVTQTINDFYFVI | Rv0987 |
| 191 | INLIIHYVHRAGALG | Rv2996c |
| 192 | IQARAAALAFEQAYA | Rv3136 |
| 193 | IRQLERLLQAVVGAG | Rv1317c |
| 194 | LGGLWTAVSPHLSPL | Rv1196 |
| 195 | LSPISNMVSMANNHV | Rv1196 |
| 196 | LSPLSNMVSMANNHM | Rv1196 |
| 197 | PSPSMGRDIKVQFQS | Rv3804c |
| 198 | QMATTLPVQRHPRSL | Rv2031c |
| 199 | QVPSASMGRDIKVQF | Rv0129c |
| 200 | RCALHWFPGSHLLHV | Rv0293c |
| 201 | RSPISNMVSMANNHM | Rv1196 |
| 202 | SARLRLLRDRLVEGV | Rv3025c |
| 203 | STIFPFRRLFMVADV | Rv0294 |
| 204 | AAAQASAAAAAYEAA | Rv1802 |
| 205 | AAFSRMLSLFFRQHI | Rv2823c |
| 206 | AASLLDEDMDALEEA | Rv3015c |
| 207 | AATQARAAAAAFEAA | Rv1789 |
| 208 | AAVDKDAVIVAAAGN | Rv0291 |
| 209 | AEAPAAAAAPEEQVQ | Rv0256c |
| 210 | AEHQAIISDVLTASD | Rv3619c |
| 211 | AILRRRRRIAEPATC | Rv0292 |
| 212 | ALSRVQSMFLGTGGS | Rv1172c |
| 213 | APQINFFYYLGEPIV | Rv1172c |
| 214 | APYVAWMRATAIQAE | Rv1705c |
| 215 | AQAVYDFRSIVDYLR | Rv0293c |
| 216 | AQLGYTIRQLERLLQ | Rv1317c |
| 217 | AQNGVQAMSSLGSSL | Rv1196 |
| 218 | ARILRQLATPISVII | Rv2024c |
| 219 | ARTDLLAFTAFPKQI | Rv3115 |
| 220 | ATEVVRRLTATAHRG | Rv0291 |
| 221 | AWMSAAAAQAEQAAT | Rv1789 |
| 222 | CILAWILVRIINVRS | Rv0987 |
| 223 | DPMVQIPRLVANNTR | Rv0129c |
| 224 | EHELYVAVLSNALHR | Rv0299 |
| 225 | FDHEFTFGWDELLSK | Rv2823c |
| 226 | FDREFTFGWDELLSK | Rv2823c |
| 227 | FPDRASIIRLVGAVL | Rv1199c |
| 228 | FYNEKAFLLTTFDVS | Rv2823c |
| 229 | GWSSLGREYAAVAEE | Rv0453 |
| 230 | HPQQFIYAGSLSALL | Rv1886c |
| 231 | IMLLAYYIAAVNIES | Rv2024c |
| 232 | INLIIHYVDRPGALG | Rv2996c |
| 233 | IVQINGRHFDLRAQG | Rv2996c |
| 234 | LAWLVQASANSAAMA | Rv0256c |
| 235 | LDYLRRMTVFLQGLM | Rv0293c |
| 236 | LLEFAVVLELAILSI | Rv3021c |
| 237 | LQSLGADIASEQAVL | Rv3019c |
| 238 | MAFLRSVSCLAAAVF | Rv3330 |
| 239 | MAFLRSVSRLAAAVF | Rv3330 |
| 240 | MSFVTTQPEALAAAA | Rv1788 |
| 241 | NRASLMQLISTNVFG | Rv1789 |
| 242 | RGKVVLIDFWAYPCI | Rv2874 |
| 243 | RSRPRRTTRRMDRRT | Rv1199c |
| 244 | SSYAATEVANAAAAS | Rv1195 |
| 245 | SSYAATEVANAAAGQ | Rv1195 |
| 246 | STIFPFRRLFMVAEV | Rv0294 |
| 247 | VTANRAELKALIASN | Rv3136 |
| 248 | WFINWYLPISQLFYN | Rv1705c |
| 249 | YAAALVAMPTLAELA | Rv0453 |
| 250 | YASVEAANASPLQVA | Rv2853 |

TABLE 3
ATB/LTBI shared

| SEQ | | |
|---|---|---|
| ID | | ORF from |
| NO: | Sequence | prediction |
| 251 | TPVQSQRVDPSAASG | Rv2094c |
| 252 | MAKTIAYDEEARRGL | Rv0440 |
| 253 | NQAFRNIVNMLHGVR | Rv3620c |
| 254 | VDLAKSLRIAAKIYS | Rv3615c |
| 255 | LRIAAKIYSEADEAW | Rv3615c |
| 256 | AAFQGAHARFVAAAA | Rv3020c |
| 257 | AVLVATNFFGINTIP | Rv3018c |
| 258 | AYGSFVRTVSLPVGA | Rv2031c |
| 259 | MSQIMYNYPAMMAHA | Rv3019c |
| 260 | AAFQAAHARFVAAAA | Rv0287 |
| 261 | AAGTYVAADAAAASS | Rv3020c |
| 262 | LQSLGAEIAVEQAAL | Rv0288 |
| 263 | AEHQAIIRDVLTASD | Rv1198 |
| 264 | MSQIMYNYPAMRAHA | Rv3019c |
| 265 | IPVMAYLVGLFAWVL | Rv0290 |
| 266 | AAGTYVAADAAAAST | Rv0287 |
| 267 | GINTIPIAINEAEYV | Rv1387 |
| 268 | GVLVATNFFGINTIP | Rv0280 |
| 269 | VHVSFVMAYPEMLAA | Rv1195 |
| 270 | AAASWDALAAELASA | Rv3135 |
| 271 | AAGGWDSLAAELATT | Rv3136 |
| 272 | EDLVRAYHAMSSTHE | Rv0288 |
| 273 | EDLVRAYHSMSSTHE | Rv0288 |
| 274 | FFGQNTAAIAATEAQ | Rv3136 |
| 275 | LLGQNTAAIAAIEAQ | Rv3136 |
| 276 | AAFSKLPASTIDELK | Rv2875 |
| 277 | GWYLVAATAAAATLR | Rv0290 |
| 278 | KQQVIAELYEKFFRI | Rv2024c |
| 279 | QLSALWARFPLPVIP | Rv0290 |
| 280 | AMMARDTAEAAKWGG | Rv0288 |
| 281 | KQELDEISTNIRQAG | Rv3874 |
| 282 | MAMMARDTAEAAKWG | Rv0288 |
| 283 | ISEAGQAMASTEGNV | Rv3875 |
| 284 | QKWDATATELNNALQ | Rv3875 |
| 285 | YQGVQQKWDATATEL | Rv3875 |
| 286 | AQWNQAMEDLVRAYH | Rv0288 |
| 287 | EISTNIRQAGVQYSR | Rv3874 |
| 288 | IRQAGVQYSRADEEQ | Rv3874 |
| 289 | NLARTISEAGQAMAS | Rv3875 |
| 290 | AAGTAAQAAVVRFQE | Rv3874 |
| 291 | HEANTMAMMARDTAE | Rv0288 |
| 292 | MSQIMYNYPAMLGHA | Rv0288 |
| 293 | MTEQQWNFAGIEAAA | Rv3875 |
| 294 | SAWQGDTGITYQAWQ | Rv0288 |
| 295 | AMEDLVRAYHAMSST | Rv0288 |
| 296 | DTGITYQAWQAQWNQ | Rv0288 |
| 297 | GDMAGYAGTLQSLGA | Rv0288 |
| 298 | GSASLVAAAQMWDSV | Rv1196 |
| 299 | INSARMYAGPGSASL | Rv1196 |
| 300 | LIATNLLGQNTPAIA | Rv1196 |
| 301 | LLPFEEAPEMTSAGG | Rv1196 |
| 302 | MAEMKTDAATLAQEA | Rv3874 |
| 303 | MGRDIKVQFQSGGAN | Rv3804c |
| 304 | NNALQNLARTISEAG | Rv3875 |
| 305 | NVTSIHSLLDEGKQS | Rv3875 |
| 306 | PSMGRDIKVQFQSGG | Rv1886c |
| 307 | QAALQSAWQGDTGIT | Rv0288 |
| 308 | QVESTAGSLQGQWRG | Rv3874 |
| 309 | SAIQGNVTSIHSLLD | Rv3875 |
| 310 | SGSEAYQGVQQKWDA | Rv3875 |
| 311 | TPAIAVNEAEYGEMW | Rv1196 |
| 312 | VRAYHAMSSTHEANT | Rv0288 |
| 313 | YAGTLQSLGAEIAVE | Rv0288 |
| 314 | YNYPAMLGHAGDMAG | Rv0288 |
| 315 | YQAWQAQWNQAMEDL | Rv0288 |

TABLE 4
MTB300 + ATB-specific

| | | ORF |
|---|---|---|
| | | from |
| | Sequence | prediction |
| 316 | ILLLVAVVALLFTSR | Rv1888c |
| 317 | EATGLLVALRALADI | Rv2095c |
| 318 | ACLLGLTILLLAVNR | Rv0176 |
| 319 | AEMKTDAATLAQEAG | Rv3874 |
| 320 | AIFMGCYLRFLRPGR | Rv3063 |
| 321 | AQHRHRYLTMVNVGR | Rv1351 |
| 322 | GWLVLIAVLALSLVR | Rv3635 |
| 323 | ILVGAAAAVVLVAMR | Rv2051c |
| 324 | IRRLTAAVALAAAGA | Rv0176 |
| 325 | IVVVAVLALGRRIPP | Rv1508c |
| 326 | LIGFALLAFCSVVAR | Rv1349 |
| 327 | AAAAAYEAAFAATVP | Rv1807 |
| 328 | AAEVLKILLGHGRVY | Rv2338c |
| 329 | AAIHDQFVATLASSA | Rv1195 |
| 330 | AAIQARAAATAFEAA | Rv3125c |
| 331 | AAVLVLFTLIFFLYG | Rv0205 |
| 332 | ADADIIMLAPSNPVV | Rv3261 |
| 333 | ADAQYPASTAFLATT | Rv0144 |
| 334 | ADGLRADPRNQRVLL | Rv0016c |
| 335 | AGAQAEGAAAAYEAA | Rv1807 |
| 336 | AGGIYGDFFNFYLCD | Rv0590A |
| 337 | AGHTHYLIIDDVDQV | Rv0284 |
| 338 | AILLVLTVLQLRITH | Rv2040c |
| 339 | ALCLIFIIMLIITRS | Rv0676c |
| 340 | ALCLIFIIMLITTRS | Rv0676c |
| 341 | ANRWIILGVSAQAIA | Rv1997 |
| 342 | APVMWALSASLGWIL | Rv1227c |
| 343 | AQEWLRFGWFFLATR | Rv3782 |
| 344 | AQEWLRFGWFFLVTR | Rv3782 |
| 345 | ARFFGRNVNVPLMII | Rv3071 |
| 346 | ARILLLVPSISLLSQ | Rv2024c |
| 347 | ASGPKVVIDGKDQNV | Rv3763 |
| 348 | ATRFRPIIRLTVEWL | Rv3887c |
| 349 | ATRVRPIIRLTVEWL | Rv3887c |
| 350 | AVGFTPEQLEIELNW | Rv1894c |
| 351 | AVLRRLEWHFTLVFA | Rv3436c |
| 352 | DIKVQFQSGGNNSPA | Rv1886c |
| 353 | DLVWDFRLPRVVSIN | Rv3432c |
| 354 | DQLVCRVVVPAVATT | Rv0107c |
| 355 | DQLVRRVVVPAVATT | Rv0107c |
| 356 | DRYINWAKDQPQYPY | Rv3784 |
| 357 | DSQKLLAWQTTNASM | Rv0040c |
| 358 | EATGLLVALRALANI | Rv2095c |
| 359 | EDALRLLSLPRVVGV | Rv3646c |
| 360 | EDAVRNAKAAVEEGI | Rv0440 |
| 361 | EDLVRAYHAMSRTHE | Rv0288 |
| 362 | EDQVRQAIQSLDIAI | Rv0361 |
| 363 | ELWVGFTAVSALLIL | Rv0876c |
| 364 | EQDLRYLRGLGQFSD | Rv0573c |
| 365 | ERLVNLVIALLSTRG | Rv2096c |
| 366 | ESLRLYDSSYHAELF | Rv2032 |
| 367 | ESPVSYHAFDPELQL | Rv0276 |
| 368 | EVFLVIDNLYGFGRD | Rv0284 |
| 369 | FCLFGVLFVLLRRGR | Rv3794 |
| 370 | FFPFVLLLAATALYR | Rv0284 |
| 371 | FGEVIYVSAELKQKH | Rv0731c |
| 372 | FNLMLWDLDRRMRRG | Rv0276 |
| 373 | FPIFAVTHCRDVVVA | Rv1894c |
| 374 | FRAFYALPAENFKTR | Rv1629 |
| 375 | GAAAAYEAAFAATVP | Rv1807 |
| 376 | GAMIRAQAGLLEAEH | Rv2346c |
| 377 | GGLLARFPGFYIPFL | Rv0160c |
| 378 | GIDEISDNASVLVSV | Rv3493c |
| 379 | GIGRPARQRTTTYAL | Rv1173 |
| 380 | GILIVGMIVALVATG | Rv0284 |
| 381 | GITVADNVAAFSELG | Rv0694 |
| 382 | GLSSVFMAILNTRNM | Rv3910 |
| 383 | GLSSVFMAILNTRNV | Rv3910 |
| 384 | GLTAILMLYSIVIIR | Rv0017c |
| 385 | GNGYRLTGKLHIRGK | Rv1890c |
| 386 | GPILLYVLLPLLDLR | Rv3252c |
| 387 | GSTADFTGTTLNSLR | Rv3818 |
| 388 | GSWLLDLTAIALRES | Rv1122 |
| 389 | ILLLVAVVALLFTGR | Rv1888c |
| 390 | ILLRDALHATAVRIL | Rv3383c |
| 391 | IRLALSLGFRVRVAA | Rv1681 |
| 392 | IRRLLPALVVVLAGC | Rv1565c |
| 393 | ISAERYESLMEELED | Rv0268c |
| 394 | ISPQTLFFPFVLLLA | Rv0284 |
| 395 | IVDYYNFFLSGGFLA | Rv3796 |
| 396 | KAWHQRTPARAEQVA | Rv1813c |
| 397 | KDDIFYYVYGLLHDP | Rv2024c |
| 398 | KTPEWAAALSGLAAG | Rv1442 |
| 399 | LGTAAGVLLIGLVRW | Rv1111c |
| 400 | LHRAFAYTSNIFIRD | Rv3796 |
| 401 | LIIDDVDQVPDSPAM | Rv0284 |
| 402 | LILFAIVSVVAIVVL | Rv0174 |
| 403 | LPFVILIGLIVSRQW | Rv0585c |
| 404 | LPLRRLLGLVAAGLD | Rv0290 |
| 405 | LPTVRPIVAAVAERG | Rv1599 |
| 406 | LRRNSANLLVLAGAQ | Rv3365c |
| 407 | LSRAGLFFVPLAVAA | Rv0267 |
| 408 | LVYRFIRDTTWVSVR | Rv3557c |
| 409 | MLMPCFAQLYDELGI | Rv3522 |
| 410 | NRGYVLSQPGLRKLL | Rv3782 |
| 411 | NVPSYRVSQYDIVDV | Rv3458c |
| 412 | NVRDQVFNLFEVLGV | Rv0244c |
| 413 | PDWVNTIFLSTRFRA | Rv3818 |
| 414 | QLYQYRFTTVAELRR | Rv0235c |
| 415 | QSGFIAAAVLLSVLG | Rv0290 |
| 416 | RATFRALGSTGHRFL | Rv0836c |
| 417 | RILFAFDPARQAILL | Rv3182 |
| 418 | SMLFLPVRILTSPIT | Rv3125c |
| 419 | SPPGLPVAAVAEQAP | Rv2095c |
| 420 | SSAGLMVAAASPYVA | Rv1196 |
| 421 | SSWRPVLLIPLTFAL | Rv3635 |
| 422 | TATATLLPFEEAPEM | Rv1196 |
| 423 | TTPSVLFLLLKTIAT | Rv1005c |
| 424 | TVIILFLAGAVVNLK | Rv3645 |
| 425 | VLMMELNRISSHLVA | Rv3148 |
| 426 | WPLLIFWLSYTGHRH | Rv1565c |
| 427 | | |
| 428 | YLYPIVALRIRGIAL | Rv0276 |
| 429 | AMMARDTAEAAKWGG | Rv0288 |
| 430 | KQELDEISTNIRQAG | Rv3874 |
| 431 | MAMMARDTAEAAKWG | Rv0288 |
| 432 | VDLAKSLRIAAKIYS | Rv3615c |
| 433 | ISEAGQAMASTEGNV | Rv3875 |
| 434 | LRIAAKIYSEADEAW | Rv3615c |
| 435 | QKWDATATELNNALQ | Rv3875 |
| 436 | YQGVQQKWDATATEL | Rv3875 |
| 437 | AAFQGAHARFVAAAA | Rv3020c |
| 438 | AQWNQAMEDLVRAYH | Rv0288 |
| 439 | AVLVATNFFGINTIP | Rv3018c |
| 440 | AYGSFVRTVSLPVGA | Rv2031c |
| 441 | EISTNIRQAGVQYSR | Rv3874 |
| 442 | IRQAGVQYSRADEEQ | Rv3874 |
| 443 | NLARTISEAGQAMAS | Rv3875 |
| 444 | AAFQAAHARFVAAAA | Rv0287 |
| 445 | AAGTAAQAAVVRFQE | Rv3874 |
| 446 | AAGTYVAADAAAASS | Rv3020c |
| 447 | AEHQAIIRDVLTASD | Rv1198 |
| 448 | HEANTMAMMARDTAE | Rv0288 |
| 449 | LQSLGAEIAVEQAAL | Rv0288 |
| 450 | MSQIMYNYPAMLGHA | Rv0288 |
| 451 | MSQIMYNYPAMMAHA | Rv3019c |
| 452 | MTEQQWNFAGIEAAA | Rv3875 |
| 453 | SAWQGDTGITYQAWQ | Rv0288 |
| 454 | AAASWDALAAELASA | Rv3135 |
| 455 | AAFSKLPASTIDELK | Rv2875 |
| 456 | AAGGWDSLAAELATT | Rv3136 |
| 457 | AAGTYVAADAAAAST | Rv0287 |
| 458 | AMEDLVRAYHAMSST | Rv0288 |
| 459 | DTGITYQAWQAQWNQ | Rv0288 |
| 460 | EDLVRAYHAMSSTHE | Rv0288 |
| 461 | EDLVRAYHSMSSTHE | Rv0288 |
| 462 | FFGQNTAAIAATEAQ | Rv3136 |
| 463 | GDMAGYAGTLQSLGA | Rv0288 |
| 464 | GINTIPIAINEAEYV | Rv1387 |
| 465 | GSASLVAAAQMWDSV | Rv1196 |
| 466 | GVLVATNFFGINTIP | Rv0280 |
| 467 | GWYLVAATAAAATLR | Rv0290 |
| 468 | INSARMYAGPGSASL | Rv1196 |
| 469 | IPVMAYLVGLFAWVL | Rv0290 |
| 470 | KQQVIAELYEKFFRI | Rv2024c |
| 471 | LIATNLLGQNTPAIA | Rv1196 |
| 472 | LLGQNTAAIAAIEAQ | Rv3136 |
| 473 | LLPFEEAPEMTSAGG | Rv1196 |
| 474 | MAEMKTDAATLAQEA | Rv3874 |
| 475 | MGRDIKVQFQSGGAN | Rv3804c |
| 476 | MSQIMYNYPAMRAHA | Rv3019c |
| 477 | NNALQNLARTISEAG | Rv3875 |
| 478 | NVTSIHSLLDEGKQS | Rv3875 |
| 479 | PSMGRDIKVQFQSGG | Rv1886c |
| 480 | QAALQSAWQGDTGIT | Rv0288 |
| 481 | QLSALWARFPLPVIP | Rv0290 |
| 482 | QVESTAGSLQGQWRG | Rv3874 |
| 483 | SAIQGNVTSIHSLLD | Rv3875 |
| 484 | SGSEAYQGVQQKWDA | Rv3875 |
| 485 | TPAIAVNEAEYGEMW | Rv1196 |
| 486 | VHVSFVMAYPEMLAA | Rv1195 |
| 487 | VRAYHAMSSTHEANT | Rv0288 |
| 488 | YAGTLQSLGAEIAVE | Rv0288 |
| 489 | YNYPAMLGHAGDMAG | Rv0288 |
| 490 | YQAWQAQWNQAMEDL | Rv0288 |
| 491 | AAAQASAAAAAYEAA | Rv1802 |
| 492 | AAERGPGQMLGGLPV | Rv1196 |
| 493 | AAFSRMLSLFFRQHI | Rv2823c |
| 494 | AAGAQLLWQLPLLSI | Rv0290 |
| 495 | AAGVAAWSLIALMIP | Rv0290 |
| 496 | AAIHEMFVNTLQMSS | Rv1788 |
| 497 | AAIHEMFVNTLVASS | Rv1791 |
| 498 | AALPAVGAAAGAPAA | Rv3021c |
| 499 | AALPLLFFALAGQRI | Rv2874 |
| 500 | AANQLMNNVPQALQQ | Rv1196 |
| 501 | AARLLSIRAMSTKFS | Rv0291 |
| 502 | AASLLDEDMDALEEA | Rv3015c |
| 503 | AATQARAAAAAFEAA | Rv1789 |
| 504 | AAVDKDAVIVAAAGN | Rv0291 |
| 505 | AAVEEASDTAAANQL | Rv1196 |
| 506 | AAVPAVGAAAGAPAA | Rv3021c |
| 507 | AAVVRFQEAANKQKQ | Rv3874 |
| 508 | AAYETAYGLTVPPPV | Rv1196 |
| 509 | ADEEQQQALSSQMGF | Rv3874 |
| 510 | ADYLRMWIQAATVMS | Rv2123 |
| 511 | AEAPAAAAAPEEQVQ | Rv0256c |
| 512 | AEHQAIISDVLTASD | Rv3619c |
| 513 | AEHQAIVRDVLAAGD | Rv1198 |
| 514 | AEHQAIVRDVLAASD | Rv1198 |
| 515 | AEKFKEDVINDFVSS | Rv3024c |
| 516 | AELMILIATNLLGQN | Rv1361c |
| 517 | AGCQTYKWETFLTSE | Rv3804c |
| 518 | AGRFEVHAQTVEDEA | Rv3620c |
| 519 | AGSLEAEHQAIISDV | Rv3619 |
| 520 | AGSLQGQWRGAAGTA | Rv3874 |
| 521 | AGSLSALLDPSQGMG | Rv1886c |
| 522 | AGWLAFFRDLVARGL | Rv3115 |
| 523 | AHGETVSAVAELIGD | Rv3024c |
| 524 | AILRRRRRIAEPATC | Rv0292 |
| 525 | AKLMRDIPFRVGAVV | Rv0294 |
| 526 | ALGATPNTGPAPQGA | Rv3804c |
| 527 | ALLPRAGAAAAAALP | Rv1172c |
| 528 | ALPPEINSARMYAGP | Rv1196 |
| 529 | ALQSHDDVALVSVMW | Rv3025c |
| 530 | ALSAEYAAVAQELSV | Rv3022c |
| 531 | ALSRVHSMFLGTGGS | Rv1172c |
| 532 | ALSRVQSMFLGTGGS | Rv1172c |
| 533 | ALSVLVGLTAATVAI | Rv0291 |
| 534 | ALTALIRDPPADSTG | Rv0292 |
| 535 | AMRDMAGRFEVHAQT | Rv3620c |
| 536 | ANATVYMIDSVLMPP | Rv2875 |
| 537 | APQINFFYYLGEPIV | Rv1172c |
| 538 | APYVAWMRATAIQAE | Rv1705c |
| 539 | AQAAVVRFQEAANKQ | Rv3874 |
| 540 | AQAVYDFRSIVDYLR | Rv0293c |
| 541 | AQIYQAVSAQAAAIH | Rv1788 |
| 542 | AQLGYTIRQLERLLQ | Rv1317c |
| 543 | AQLSQLISLLPSTLQ | Rv1808 |
| 544 | AQNGVQAMSSLGSSL | Rv1196 |
| 545 | ARILRQLATPISVII | Rv2024c |
| 546 | ARMWIQAATTMASYQ | Rv0256c |
| 547 | ARTDLLAFTAFPKQI | Rv3115 |
| 548 | ASIIRLVGAVLAEQH | Rv3115 |
| 549 | ATEVVRRLTATAHRG | Rv0291 |
| 550 | ATSLDTMTQMNQAFR | Rv3620c |
| 551 | AVDGRFAVPQILGDE | Rv3012c |
| 552 | AVHVWLRLPAGRVEI | Rv1366 |
| 553 | AVPLRLLGGLHRMVL | Rv0690c |
| 554 | AWHGPASLAMTRAAS | Rv2608 |
| 555 | AWMSAAAAQAEQAAT | Rv1789 |
| 556 | AYAQRVYQANRAAGS | Rv0298 |
| 557 | CILAWILVRIINVRS | Rv0987 |
| 558 | DAHGAMIRAQAGSLE | Rv3619 |
| 559 | DALSGLGLPPPWQPA | Rv2608 |
| 560 | DESWQQFRQELIPLL | Rv0294 |
| 561 | DPMVQIPRLVANNTR | Rv0129c |
| 562 | DRWLDLRYVGPASAD | Rv0289 |
| 563 | DVDWAEVAADLQQGA | Rv2608 |
| 564 | DYVRMWVQAATAMSA | Rv3018c |
| 565 | DYVRMWVQAATVMSA | Rv3018c |
| 566 | EGKQSLTKLAAAWGG | Rv3875 |
| 567 | EHELYVAVLSNALHR | Rv0299 |
| 568 | EIGWEAGTAAPDEIP | Rv0292 |
| 569 | EIVQFLEETFAAYDQ | Rv3018c |
| 570 | ELFVAAYVPYVAWLV | Rv3022c |
| 571 | EQANAHGQKVQAAGN | Rv3619 |
| 572 | EQQWNFAGIEAAASA | Rv3875 |
| 573 | EVVDYLGIPASARPV | Rv0289 |
| 574 | FDHEFTFGWDELLSK | Rv2823c |
| 575 | FDREFTFGWDELLSK | Rv2823c |
| 576 | FGFSIPQLGFTLSGA | Rv2608 |
| 577 | FGLFPDVDWAEVAAD | Rv2608 |
| 578 | FGQNTASIAATEAQY | Rv1705c |
| 579 | FGQNTGAIAAAEARY | Rv1802 |
| 580 | FGQNTSAIAAAEAQY | Rv1789 |
| 581 | FPDRASIIRLVGAVL | Rv1199c |
| 582 | FQVIYEQANAHGQKV | Rv3619 |
| 583 | FVQALTTAAASYASV | Rv2853 |
| 584 | FYNEKAFLLTTFDVS | Rv2823c |
| 585 | GADATAAAAFEQFLA | Rv2024c |
| 586 | GAMVATNFFGINTIP | Rv0453 |
| 587 | GCQTYKWETFLTSEL | Rv1886c |
| 588 | GEEYLILSARDVLAV | Rv3418c |
| 589 | GGHNGVFDFPDSGTH | Rv3804c |
| 590 | GGLPVGQMGARAGGG | Rv1196 |
| 591 | GGQSSFYSDWYQPAC | Rv3804c |
| 592 | GILTRFGFSIPQLGF | Rv2608 |
| 593 | GKAGCQTYKWETFLT | Rv3804c |
| 594 | GLAGDAWHGPASLAM | Rv2608 |
| 595 | GNFERISGDLKTQID | Rv3874 |
| 596 | GNGVVALRNAQLVTF | Rv0289 |
| 597 | GQNYTYKWETFLTRE | Rv0129c |
| 598 | GQWRGAAGTAAQAAV | Rv3874 |
| 599 | GTVVLTATFALGAAL | Rv2874 |
| 600 | GWIISNIFGAIPVLA | Rv3021c |
| 601 | GWIISNIFGAIPVLG | Rv3021c |
| 602 | GWSSLGREYAAVAEE | Rv0453 |
| 603 | HGQKVQAAGNNMAQT | Rv3619 |
| 604 | HPQQFIYAGSLSALL | Rv1886c |
| 605 | IAENRAELMILIATN | Rv1196 |
| 606 | IDLNVLLSAAINFFL | Rv0985c |
| 607 | IGLVTQTINDFYFVI | Rv0987 |
| 608 | ILPIAEMSVVAMEFG | Rv3025c |
| 609 | IMLLAYYIAAVNIES | Rv2024c |
| 610 | INLIIHYVDRPGALG | Rv2996c |
| 611 | INLIIHYVHRAGALG | Rv2996c |
| 612 | IQARAAALAFEQAYA | Rv3136 |
| 613 | IRQLERLLQAVVGAG | Rv1317c |
| 614 | IVQINGRHFDLRAQG | Rv2996c |
| 615 | KTQIDQVESTAGSLQ | Rv3874 |
| 616 | LAAAAAWDALAAELY | Rv1808 |
| 617 | LALVGFLGGLITGIS | Rv2874 |
| 618 | LALVGFLGGLITGTS | Rv2874 |
| 619 | LAQEAGNFERISGDL | Rv3874 |
| 620 | LAQPTQGTTPSSKLG | Rv1196 |
| 621 | LAWLVQASANSAAMA | Rv0256c |
| 622 | LDYLRRMTVFLQGLM | Rv0293c |
| 623 | LENDNQLLYNYPGAL | Rv3330 |
| 624 | LGGLWTAVSPHLSPL | Rv1196 |
| 625 | LGLPPPWQPALPRLF | Rv2608 |
| 626 | LHGVRDGLVRDANNY | Rv3620c |
| 627 | LLEFAVVLELAILSI | Rv3021c |
| 628 | LLEQAAAVEEASDTA | Rv1196 |
| 629 | LLGQNTPAIAVNEAE | Rv1196 |
| 630 | LNYRPLLPKDRRMII | Rv0293c |
| 631 | LPPEVNSARIFAGAG | Rv2608 |
| 632 | LQSLGADIASEQAVL | Rv3019c |
| 633 | LQSLWANFYELLADA | Rv1366 |
| 634 | LRGLLSTFIAALMGA | Rv3115 |
| 635 | LSIVMPVGGQSSFYS | Rv1886c |
| 636 | LSPISNMVSMANNHM | Rv1196 |
| 637 | LSPISNMVSMANNHV | Rv1196 |
| 638 | LSPLSNMVSMANNHM | Rv1196 |
| 639 | LTKLAAAWGGSGSEA | Rv3875 |
| 640 | LTSELPQWLSANRAV | Rv1886c |
| 641 | LTTYILLPSQDLPLL | Rv2608 |
| 642 | MAFLRSVSCLAAAVF | Rv3330 |
| 643 | MAFLRSVSRLAAAVF | Rv3330 |
| 644 | MHVSFVMAYPEMLAA | Rv1195 |
| 645 | MLGHAGDMAGYAGTL | Rv0288 |
| 646 | MNFAVLPPEVNSARI | Rv2608 |
| 647 | MSFVTTQPEALAAAA | Rv1788 |
| 648 | MTDPHAMRDMAGRFE | Rv3620c |
| 649 | MTINYQFGDVDAHGA | Rv2346c |
| 650 | MTSRFMTDPHAMRDM | Rv3620c |
| 651 | MVAAASPYVAWMSVT | Rv1196 |
| 652 | MVDFGALPPEINSAR | Rv1196 |
| 653 | MWDPDVYLAFSGHRN | Rv0294 |
| 654 | MYRELLELVAADVES | Rv0690c |
| 655 | NIVNMLHGVRDGLVR | Rv3620c |
| 656 | NRASLMQLISTNVFG | Rv1789 |
| 657 | PGQMLGGLPVGQMGA | Rv1196 |
| 658 | PQLGFTLSGATPADA | Rv2608 |
| 659 | PQVVNINTKLGYNNA | Rv0125 |
| 660 | PQWLSANRAVKPTGS | Rv1886c |
| 661 | PSPSMGRDIKVQFQS | Rv3804c |
| 662 | PVGGQSSFYSDWYSP | Rv1886c |
| 663 | QAAGNNMAQTDSAVG | Rv3619 |
| 664 | QAMASTEGNVTGMFA | Rv3875 |
| 665 | QASPDLLRGLLSTFI | Rv3115 |
| 666 | QFGDVDAHGAMIRAQ | Rv3619 |
| 667 | QLGRNFQVIYEQANA | Rv3619 |
| 668 | QMATTLPVQRHPRSL | Rv2031c |
| 669 | QQIKFAALSARAVAL | Rv3024c |
| 670 | QRNLSVVAPSQFTFS | Rv2660c |
| 671 | QTYKWETFLTSELPG | Rv3804c |
| 672 | QVPSASMGRDIKVQF | Rv0129c |
| 673 | RCALHWFPGSHLLAC | Rv0293c |
| 674 | RCALHWFPGSHLLHV | Rv0293c |
| 675 | RGKVVLIDFWAYPCI | Rv2874 |
| 676 | RQSGATIADVLAEKE | Rv3876 |
| 677 | RRMWASAQNISGAGW | Rv3620c |
| 678 | RSPISNMVSMANNHM | Rv1196 |
| 679 | RSRPRRTTRRMDRRT | Rv1199c |
| 680 | RVPEDLLAMVVAVEQ | Rv0299 |
| 681 | SAMILAAYHPQQFIY | Rv1886c |
| 682 | SAQNISGAGWSGMAE | Rv3620c |
| 683 | SARLRLLRDRLVEGV | Rv3025c |
| 684 | SPAMVAANRTRLASL | Rv2608 |
| 685 | SPYVAWMSVTAGQAE | Rv1196 |
| 686 | SSFYSDWYSPACGKA | Rv1886c |
| 687 | SSYAATEVANAAAAS | Rv1195 |
| 688 | SSYAATEVANAAAGQ | Rv1195 |
| 689 | STIFPFRRLFMVADV | Rv0294 |
| 690 | STIFPFRRLFMVAEV | Rv0294 |
| 691 | SVLAAVGLGLATAPA | Rv0125 |
| 692 | TATELNNALQNLART | Rv3875 |
| 693 | TDAATLAQEAGNFER | Rv3874 |
| 694 | THSWEYWGAQLNAMK | Rv1886c |
| 695 | TRAASPYVGWLNTAA | Rv2608 |
| 696 | VAAAQMWDSVASDLF | Rv1196 |
| 697 | VAQVGPQVVNINTKL | Rv0125 |
| 698 | VEDEARRMWASAQNI | Rv3620c |
| 699 | VEYLQVPSPSMGRDI | Rv3804c |
| 700 | VHAQTVEDEARRMWA | Rv3620c |
| 701 | VPPPVIAENRAELMI | Rv1196 |
| 702 | VPSPSMGRDIKVQFQ | Rv3804c |
| 703 | VQGVNDALSGLGLPP | Rv2608 |
| 704 | VQYSRADEEQQQALS | Rv3874 |
| 705 | VRFQEAANKQKQELD | Rv3874 |
| 706 | VTANRAELKALIASN | Rv3136 |
| 707 | VTVDAAVLAAIDADA | Rv0298 |
| 708 | VVAPSQFTFSSRSPD | Rv2660c |
| 709 | WFINWYLPISQLFYN | Rv1705c |
| 710 | WMSVTAGQAELTAAQ | Rv1196 |
| 711 | WNFAGIEAAASAIQG | Rv3875 |
| 712 | YAAALVAMPTLAELA | Rv0453 |
| 713 | YASVEAANASPLQVA | Rv2853 |
| 714 | YGEMWAQDAAAMFGY | Rv1196 |
| 715 | YLGLEVLTRARAALT | Rv3115 |
| 716 | YPTVDYAFQYDGVND | Rv2608 |
| 717 | YRIAARPGAVTRRAA | Rv3015c |
| 718 | YVAWMSATAALAREA | Rv1802 |

TABLE 5
T cell epitopes recognized by
participants with ATB (mid-treatment)

| | SEQ | |
|---|---|---|
| | ID | |
| Rv number | NO: | Epitope sequence |
| Rv1038c, Rv1197, | 719 | NQAFRNIVNMLHGVR |
| Rv1792, Rv2347c, | | |
| Rv3620c | | |
| Rv1196, Rv1361c, | 720 | INSARMYAGPGSASL |
| Rv3478 | | |
| Rv0107c | 721 | DQLVRRVVVPAVATT |
| Rv0287 | 722 | AAFQAAHARFVAAAA |
| Rv0288 | 723 | AMMARDTAEAAKWGG |
| Rv0288 | 724 | AQWNQAMEDLVRAYH |
| Rv0288 | 725 | EDLVRAYHSMSSTHE |
| Rv0288 | 726 | MAMMARDTAEAAKWG |
| Rv0288, Rv3019c | 727 | GDMAGYAGTLQSLGA |
| Rv0676c | 728 | ALCLIFIIMLIITRS |
| Rv1196, Rv1361c | 729 | LIATNLLGQNTPAIA |
| Rv1196, Rv1361c | 730 | SSAGLMVAAASPYVA |
| Rv1387, Rv3018c, | 731 | AVLVATNFFGINTIP |
| Rv3021c | | |
| Rv3019c | 732 | MSQIMYNYPAMMAHA |
| Rv3136 | 733 | AAGGWDSLAAELATT |
| Rv3615c | 734 | LRIAAKIYSEADEAW |
| Rv3874 | 735 | EISTNIRQAGVQYSR |
| Rv3874 | 736 | IRQAGVQYSRADEEQ |
| Rv3874 | 737 | KQELDEISTNIRQAG |
| Rv3875 | 738 | ISEAGQAMASTEGNV |
| Rv3875 | 739 | MTEQQWNFAGIEAAA |
| Rv3875 | 740 | NLARTISEAGQAMAS |
| Rv3875 | 741 | QKWDATATELNNALQ |
| Rv3875 | 742 | YQGVQQKWDATATEL |
| Rv3887c | 743 | ATRFRPIIRLTVEWL |
| Rv0016c | 744 | ADGLRADPRNQRVLL |
| Rv0017c | 745 | GLTAILMLYSIVIIR |
| Rv0040c | 746 | DSQKLLAWQTTNASM |
| Rv0144 | 747 | ADAQYPASTAFLATT |
| Rv0160c | 748 | GGLLARFPGFYIPFL |
| Rv0174 | 749 | LILFAIVSVVAIVVL |
| Rv0176³ | 750 | ACLLGLTILLLAVNR |
| Rv01763 | 751 | IRRLTAAVALAAAGA |
| Rv0205 | 752 | AAVLVLFTLIFFLYG |
| Rv0235c | 753 | QLYQYRFTTVAELRR |
| Rv0244c | 754 | NVRDQVFNLFEVLGV |
| Rv0266c | 755 | YLRIAVANMANAVKK |
| Rv0267 | 756 | LSRAGLFFVPLAVAA |
| Rv0268c | 757 | ISAERYESLMEELED |
| Rv0276 | 758 | ESPVSYHAFDPELQL |
| Rv0276 | 759 | FNLMLWDLDRRMRRG |
| Rv0276 | 760 | YLYPIVALRIRGIAL |
| Rv0280 | 761 | GVLVATNFFGINTIP |
| Rv0284 | 762 | AGHTHYLIIDDVDQV |
| Rv0284 | 763 | EVFLVIDNLYGFGRD |
| Rv0284 | 764 | FFPFVLLLAATALYR |
| Rv0284 | 765 | GILIVGMIVALVATG |
| Rv0284 | 766 | ISPQTLFFPFVLLLA |
| Rv0284 | 767 | LIIDDVDQVPDSPAM |
| Rv0287 | 768 | AAGTYVAADAAAAST |
| Rv0288 | 769 | AMEDLVRAYHAMSST |
| Rv0288 | 770 | DTGITYQAWQAQWNQ |
| Rv0288 | 771 | EDLVRAYHAMSSTHE |
| Rv0288 | 772 | HEANTMAMMARDTAE |
| Rv0288 | 773 | LQSLGAEIAVEQAAL |
| Rv0288 | 774 | MSQIMYNYPAMLGHA |
| Rv0288 | 775 | QAALQSAWQGDTGIT |
| Rv0288 | 776 | SAWQGDTGITYQAWQ |
| Rv0288 | 777 | VRAYHAMSSTHEANT |
| Rv0288 | 778 | YAGTLQSLGAEIAVE |
| Rv0288 | 779 | YNYPAMLGHAGDMAG |
| Rv0288 | 780 | YQAWQAQWNQAMEDL |
| Rv0290 | 781 | GWYLVAATAAAATLR |
| Rv0290 | 782 | IPVMAYLVGLFAWVL |
| Rv0290 | 783 | LPLRRLLGLVAAGLD |
| Rv0290 | 784 | QLSALWARFPLPVIP |
| Rv0290 | 785 | QSGFIAAAVLLSVLG |
| Rv0361 | 786 | EDQVRQAIQSLDIAI |
| Rv0440 | 787 | EDAVRNAKAAVEEGI |
| Rv0440 | 788 | MAKTIAYDEEARRGL |
| Rv0573c | 789 | EQDLRYLRGLGQFSD |
| Rv0585c | 790 | LPFVILIGLIVSRQW |
| Rv0590A | 791 | AGGIYGDFFNFYLCD |
| Rv0694 | 792 | GITVADNVAAFSELG |
| Rv0731c | 793 | FGEVIYVSAELKQKH |
| Rv0836c | 794 | RATFRALGSTGHRFL |
| Rv0876c | 795 | ELWVGFTAVSALLIL |
| Rv1005c | 796 | TTPSVLFLLLKTIAT |
| Rv1111c | 797 | LGTAAGVLLIGLVRW |
| Rv1122 | 798 | GSWLLDLTAIALRES |
| Rv1173 | 799 | GIGRPARQRTTTYAL |
| Rv1195 | 800 | AAIHDQFVATLASSA |
| Rv1195 | 801 | VHVSFVMAYPEMLAA |
| Rv1196 | 802 | GSASLVAAAQMWDSV |
| Rv1196 | 803 | LLPFEEAPEMTSAGG |
| Rv1196 | 804 | TATATLLPFEEAPEM |
| Rv1196, Rv1361c | 805 | TPAIAVNEAEYGEMW |
| Rv1198 | 806 | AEHQAIIRDVLTASD |
| Rv1198, Rv2346c | 807 | GAMIRAQAGLLEAEH |
| Rv1227c | 808 | APVMWALSASLGWIL |
| Rv1349³ | 809 | LIGFALLAFCSVVAR |
| Rv1351³ | 810 | AQHRHRYLTMVNVGR |
| Rv1387 | 811 | GINTIPIAINEAEYV |
| Rv1442 | 812 | KTPEWAAALSGLAAG |
| Rv1508c³ | 813 | IVVVAVLALGRRIPP |
| Rv1565c | 814 | IRRLLPALVVVLAGC |
| Rv1565c | 815 | WPLLIFWLSYTGHRH |
| Rv1599 | 816 | LPTVRPIVAAVAERG |
| Rv1629 | 817 | FRAFYALPAENFKTR |
| Rv1681 | 818 | IRLALSLGFRVRVAA |
| Rv1807, Rv1802 | 819 | AAAAAYEAAFAATVP |
| Rv1807, Rv1802 | 820 | AGAQAEGAAAAYEAA |
| Rv1807, Rv1802 | 821 | GAAAAYEAAFAATVP |
| Rv1813c | 822 | KAWHQRTPARAEQVA |
| Rv1886c | 823 | DIKVQFQSGGNNSPA |
| Rv1886c, Rv3804c | 824 | PSMGRDIKVQFQSGG |
| Rv1888c³ | 825 | ILLLVAVVALLFTGR |
| Rv1888c³ | 826 | ILLLVAVVALLFTSR |
| Rv1890c | 827 | GNGYRLTGKLHIRGK |
| Rv1894c | 828 | AVGFTPEQLEIELNW |
| Rv1894c | 829 | FPIFAVTHCRDVVVA |
| Rv1997 | 830 | ANRWIILGVSAQAIA |
| Rv2024c | 831 | ARILLLVPSISLLSQ |
| Rv2024c | 832 | KDDIFYYVYGLLHDP |
| Rv2024c | 833 | KQQVIAELYEKFFRI |
| Rv2031c | 834 | AYGSFVRTVSLPVGA |
| Rv2032 | 835 | ESLRLYDSSYHAELF |
| Rv2040c | 836 | AILLVLTVLQLRITH |
| Rv2051c³ | 837 | ILVGAAAAVVLVAMR |
| Rv2094c | 838 | TPVQSQRVDPSAASG |
| Rv2095c³ | 839 | EATGLLVALRALADI |
| Rv2095c³ | 840 | EATGLLVALRALANI |
| Rv2095c³ | 841 | SPPGLPVAAVAEQAP |
| Rv2096c | 842 | ERLVNLVIALLSTRG |
| Rv2338c | 843 | AAEVLKILLGHGRVY |
| Rv2875 | 844 | AAFSKLPASTIDELK |
| Rv3019c | 845 | MSQIMYNYPAMRAHA |
| Rv3020c | 846 | AAFQGAHARFVAAAA |
| Rv3020c | 847 | AAGTYVAADAAAASS |
| Rv30633 | 848 | AIFMGCYLRFLRPGR |
| Rv3071 | 849 | ARFFGRNVNVPLMII |
| Rv3125c | 850 | AAIQARAAATAFEAA |
| Rv3125c | 851 | SMLFLPVRILTSPIT |
| Rv3135 | 852 | AAASWDALAAELASA |
| Rv3136 | 853 | FFGQNTAAIAATEAQ |
| Rv3136 | 854 | LLGQNTAAIAAIEAQ |
| Rv3148 | 855 | VLMMELNRISSHLVA |
| Rv3182 | 856 | RILFAFDPARQAILL |
| Rv3252c | 857 | GPILLYVLLPLLDLR |
| Rv3261 | 858 | ADADIIMLAPSNPVV |
| Rv3365c | 859 | LRRNSANLLVLAGAQ |
| Rv3383c | 860 | ILLRDALHATAVRIL |
| Rv3432c | 861 | DLVWDFRLPRVVSIN |
| Rv3436c | 862 | AVLRRLEWHFTLVFA |
| Rv3458c | 863 | NVPSYRVSQYDIVDV |
| Rv3493c | 864 | GIDEISDNASVLVSV |
| Rv3522 | 865 | MLMPCFAQLYDELGI |
| Rv3557c | 866 | LVYRFIRDTTWVSVR |
| Rv3615c | 867 | VDLAKSLRIAAKIYS |
| Rv3635³ | 868 | GWLVLIAVLALSLVR |
| Rv36353 | 869 | SSWRPVLLIPLTFAL |
| Rv3645 | 870 | TVIILFLAGAVVNLK |
| Rv3646c | 871 | EDALRLLSLPRVVGV |
| Rv3763 | 872 | ASGPKVVIDGKDQNV |
| Rv3782 | 873 | AQEWLRFGWFFLATR |
| Rv3782 | 874 | AQEWLRFGWFFLVTR |
| Rv3782 | 875 | NRGYVLSQPGLRKLL |
| Rv3784 | 876 | DRYINWAKDQPQYPY |
| Rv3794 | 877 | FCLFGVLFVLLRRGR |
| Rv3796 | 878 | IVDYYNFFLSGGFLA |
| Rv3796 | 879 | LHRAFAYTSNIFIRD |
| Rv3804c | 880 | MGRDIKVQFQSGGAN |
| Rv3818 | 881 | GSTADFTGTTLNSLR |
| Rv3818 | 882 | PDWVNTIFLSTRFRA |
| Rv3874 | 883 | AAGTAAQAAVVRFQE |
| Rv3874 | 884 | AEMKTDAATLAQEAG |
| Rv3874 | 885 | MAEMKTDAATLAQEA |
| Rv3874 | 886 | QVESTAGSLQGQWRG |
| Rv3875 | 887 | NNALQNLARTISEAG |
| Rv3875 | 888 | NVTSIHSLLDEGKQS |
| Rv3875 | 889 | SAIQGNVTSIHSLLD |
| Rv3875 | 890 | SGSEAYQGVQQKWDA |
| Rv3910 | 891 | GLSSVFMAILNTRNM |
| Rv3910 | 892 | GLSSVFMAILNTRNV |

1) PC85-85 peptides from the cell wall and cell processes category
2) PC71-71 peptides from all categories except cell wall and cell processes
3) Novel T cell antigens identified in this study
4) ATB116 includes 2 peptides with 1 amino acid mismatch (recognized in the screen by the same participant)

### Example 6: Hierarchy in Reactivity Against TB Vaccine and IGRA Antigens

To further evaluate T cell responses against TB vaccine candidate and IGRA antigens (see methods), the inventors tested 517 15-mer peptides overlapping by 10 amino acids spanning these antigens. Positive pools were deconvoluted to identify individual T cell epitopes. Overall, 67% of the ATB participants recognized epitopes from at least one antigen; on average, these participants recognized 2 different antigens (range 1-4).

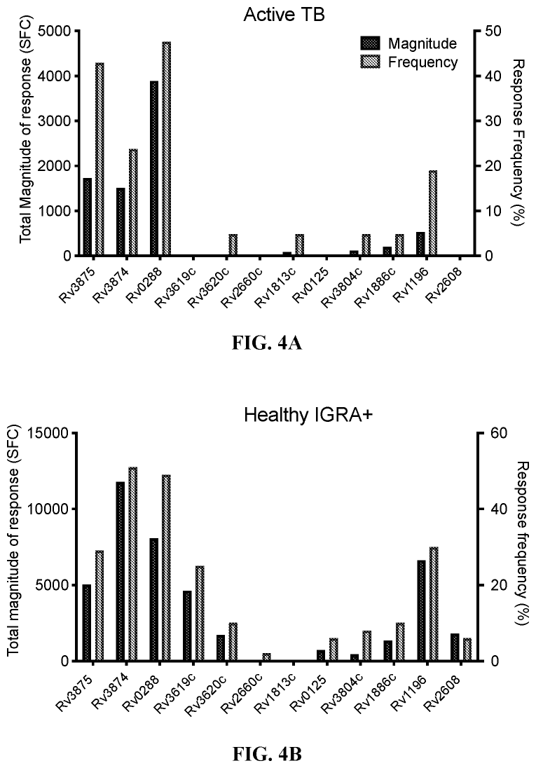

The inventors next compared the magnitude and frequency of the response for these antigens in the ATB participants, with what was previously observed in healthy IGRA+ participants from South Africa (37). The most frequently recognized antigens in ATB and IGRA+ alike were Rv0288 (TB310.4), Rv3875 (ESAT-6), Rv3874 (CFP10), and Rv1196 (PPE18) (FIG. 4A, 4B). However, some antigens were differentially recognized in the two cohorts. Specifically, Rv3875 was more frequently recognized than Rv3874 in ATB vs IGRA+ participants. The Rv1813c antigen was reactive in participants with ATB and not in IGRA+. Finally, the Rv3619c (EsxV), Rv2660c, Rv0125 (Mtb32a) and Rv2608 (PPE42) antigens were reactive in IGRA+ and completely unreactive in participants with ATB (FIG. 4A, 4B).

The proteome-wide screen detected the two most reactive vaccine antigens, Rv0288 and Rv3874. Rv3875 was not detected in the proteome-wide screen, likely due to its small size, with only 2 peptides representing it in the proteome-wide library. The results confirm that the antigens detected in the proteome-wide screen are the most frequently recognized together with Rv3875 and that the other vaccine antigens account for a small fraction of the response in this ATB cohort. In conclusion, the screen of vaccine and IGRA antigens, together with the proteome-wide screen, identified a total of 174 epitopes, which were next investigated for functionality and differential reactivity in further experiments.

### Example 7: Functionality of T Cell Responses Specific for Different Protein Antigen Categories in ATB

The responses associated with the 174 identified epitopes were characterized in more detail by intracellular cytokine secretion assays to characterize T cell responses specific to antigens from the different protein categories. One epitope pool (PC85) corresponded to epitopes from antigens in the cell wall and cell processes category, and a separate pool encompassed epitopes from other protein categories (PC71) (Table 5). As a comparator, the inventors included the previously described MTB300 pool, based on epitopes recognized in IGRA+ participants (22), which includes peptides from all functional categories.

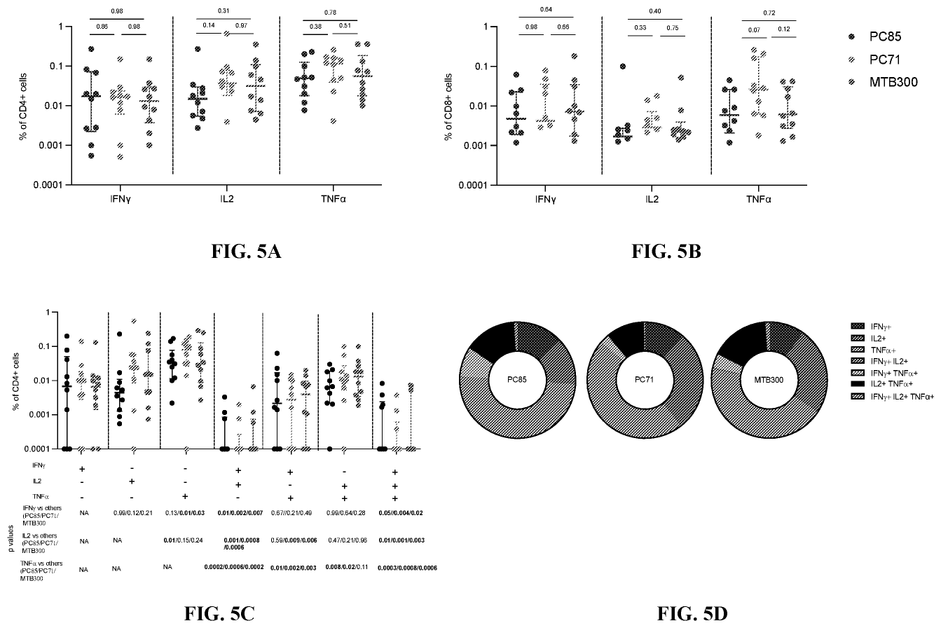

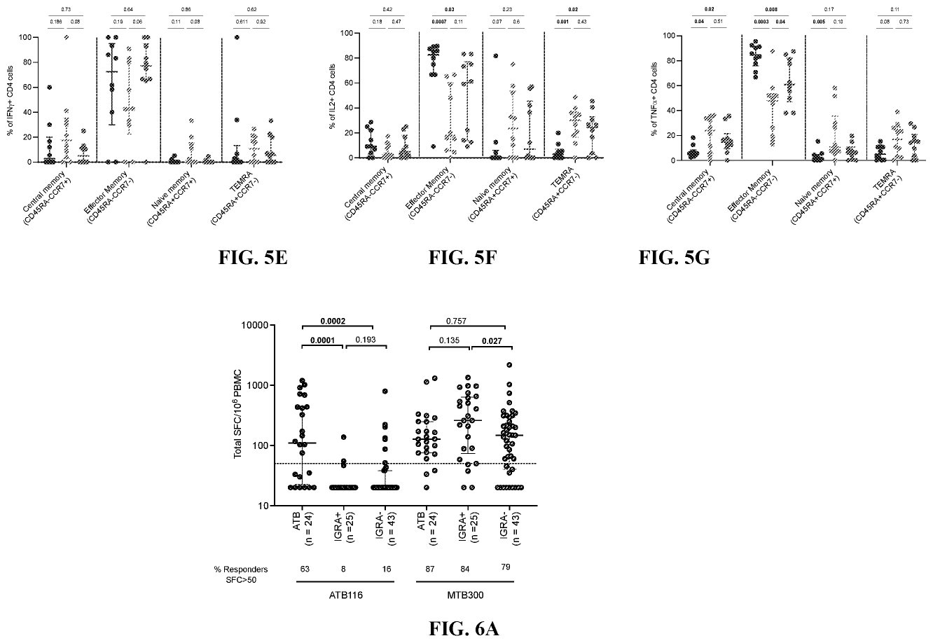

As expected, based on the peptide library design to bind HLA class II alleles, the majority of the response was meditated by CD4 T cells (FIG. 5A). Among them, the frequency of TNFα+CD4 cells was relatively higher than IFNγ (p=0.0006 for PC71) or IL-2 (p=0.04 for PC85). In addition, some responses were detected in CD8+ T cells (FIG. 5B), likely due to nested HLA class I binding epitopes within the 15-mer peptides.

Frequencies of cytokine-expressing CD4 T cells in response to both pools were similar to MTB300 (IFNγ, p=0.98 for PC85 and PC71; IL-2, p=0.31 and 0.97 for PC85 and PC71 respectively; TNFα, p=0.78 and 0.51 for PC85 and PC71 respectively) (FIG. 5A). The vast majority of cytokine producing CD4 T cells expressed a single cytokine TNFα or IFNγ, followed by TNFα+IL2+ and TNFα+IFNγ+ dual cytokine expressed cells with none to very few IFNγ+IL2+ cells and triple cytokine producing cells (FIGS. 5C and 4D). A higher frequency of single TNFα-producing T cells was present compared to other single (IFNγ p=0.01 (PC71), IL-2 p=0.01 (PC85)), dual (IFNγ+IL-2+p=0.0006 (PC71), p=0.0002 (PC85), TNFα+IFNγ+p=0.002 (PC71), p=0.01 (PC85), TNFα+IL2+p=0.02 (PC71), p=0.008 (PC85)), or triple (p=0.0008 (PC71), p=0.0003 (PC85) cytokine producing cells (FIG. 5C).

Next, the inventors characterized which T cell memory subset was responsible for the reactivity. Memory subset phenotypes were determined using antibodies to CD45RA and CCR7. The majority of the cytokine producing CD4 T cells were effector memory (CD45RA−CCR7−), and as expected few were naïve (CD45RA+CCR7+) cells (FIGS. 5E-5F). For IFNγ there was no difference between the CD4 memory subsets comparing the different peptide pools (FIG. 5E). For IL-2 there was a significantly higher frequency of effector memory T cells (p=0.0007 and 0.03) and a lower frequency of T_{EMRA} (CD45RA+CCR7+) T cells (p=0.001 and 0.02) in response to PC85 compared to the PC71 and MTB300 respectively (FIG. 5F). The PC85 stimulation also resulted in a significantly higher frequency of effector memory TNFα-producing cells (p=0.0003 and 0.008) and a lower frequency of central memory cells (p=0.04 and 0.02) than PC71 and MTB300 respectively (FIG. 5G). This finding suggests distinct differentiation of the CD4 memory subsets in response to epitope pools representing different functional categories.

### Example 8: Differential T Cell Response Against Disease-Specific Peptide Pools

The data show that certain antigens and epitopes are differentially recognized in ATB vs. healthy IGRA+ individuals. Accordingly, the inventors next tested whether reactivity to these “ATB-specific epitopes,” could differentiate individuals with ATB at diagnosis, as a cohort more relevant for diagnostic purposes vs. IGRA+ and IGRA− healthy controls. Peptides that were exclusively recognized by participants with ATB (mid-treatment) as compared to the previous studies in IGRA+ participants were pooled into an ATB-pool (ATB116) consisting of 116 peptides (Table 5). Reactivity to MTB300 was utilized as a comparator.

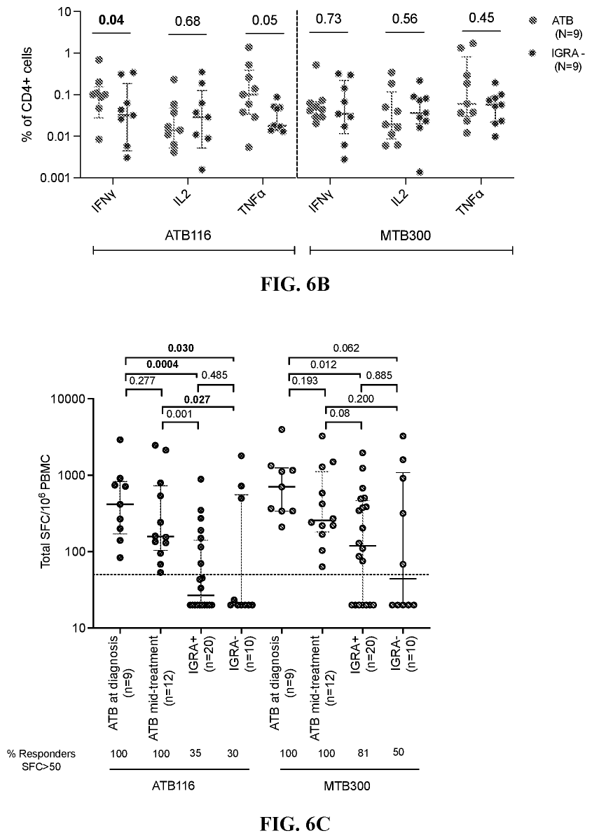

IFNγ response was determined in a Sri Lankan cohort, including 24 ATB patients (recruited at diagnosis), 25 IGRA+, and 43 IGRA− participants. ATB116 stimulation resulted in significantly higher IFNγ in patients with ATB compared to both IGRA+ and IGRA− controls (FIG. 6A), with 63% of ATB responding compared to 8% of IGRA+ and 16% of IGRA− participants. The frequency of participants responding to MTB300 was similar between the cohorts. MTB300 could not discriminate between ATB and IGRA+, but showed significantly higher magnitude of reactivity in IGRA+ compared to IGRA− (FIG. 6A). MTB300 contains epitopes that are conserved in nontuberculous mycobacteria, which have been shown to correlate with reactivity observed in IGRA− participants (39). Similar results were obtained when responses were measured by ICS rather than Fluorospot (FIG. 6B). The IFNγ, IL-2, and TNFα cytokine production was determined in 9 participants with ATB (at diagnosis) and 9 IGRA− participants from Sri Lanka. The frequency of IFNγ and TNFα producing CD4 cells against the ATB116 was significantly higher in ATB compared to IGRA-participants (FIG. 6B). No differences were observed between these cohorts in response to MTB300.

These results were independently confirmed in an independent cohort from the Republic of Moldova of ATB (at diagnosis and mid-treatment), IGRA+ and IGRA− participants (household contacts of an ATB index case). Similar to what was observed in the case of the Sri Lankan cohort, ATB116 stimulation resulted in a significantly higher magnitude of response in patients with ATB, both at diagnosis and mid-treatment (100% responders), compared to IGRA+ and IGRA− controls (35% vs. 30% responders; FIG. 6C). MTB300 did not distinguish between the different cohorts (FIG. 6C).

Next, the inventors calculated the sensitivity and specificity for ATB116 to explore its diagnostic potential. The sensitivity was calculated as the percentage of the participants with ATB responding to ATB116 out of the total ATB cohort. The specificity was calculated as the percentage of IGRA+/− who did not respond to ATB116 out of the total IGRA+/−participants. ATB116 demonstrated a high sensitivity of 62.5% and specificity of over 80% in distinguishing ATB patients from those who were IGRA+ and IGRA− in the Sri Lankan cohort. Similarly, the sensitivity was 100% and the specificity was over 60% in the Moldovan cohort differentiating ATB from IGRA+ and IGRA− participants. (Table 6).

TABLE 6
Diagnostic potential of ATB116 distinguishing patients with
ATB from IGRA+ and IGRA− participants.

| | | Sensitivity | Specificity |
|---|---|---|---|
| | | (95% CI) | (95% CI) |
| Sri Lanka | ATB vs IGRA+ | 62.5 (42.71 to | 92 (75.03 to |
| | | 78.84) | 98.58) |
| | ATB vs IGRA− | | 83.7 (70.0 to |
| | | | 91.8) |
| Moldova | ATB vs IGRA+ | 100 (84.5-100) | 65 (43.2-81.8) |
| | ATB vs IGRA− | | 70 39.6-89.2) |

### Example 9: Longitudinal Changes in Epitope Reactivity

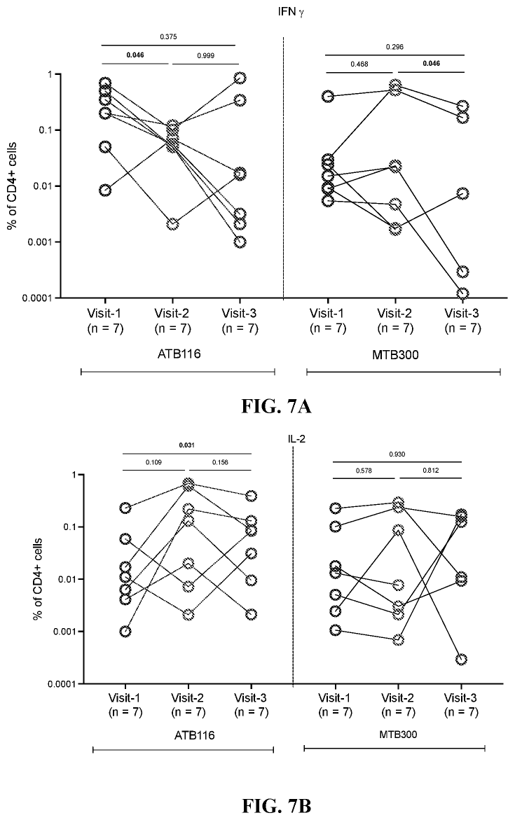

Finally, to investigate the changes in T cell response during treatment, the inventors followed 7 participants in Sri Lanka from diagnosis to mid-treatment and end of treatment. The inventors analyzed the proportion of cytokine-producing CD4 T cells after pool stimulation. In this longitudinal cohort the inventors observed a significant decrease in ATB116-specific IFNγ responses between visit 1 (at diagnosis) and visit 2 (mid-treatment) (FIG. 7A). At visit 3 (end of treatment) about half the cohort had a further decrease in their response, whereas in the other half the response against ATB116 increased again. The response against MTB300 did not change between visit 1 and 2, but was significantly decreased comparing visit 2 and 3 (FIG. 7A). The ATB116-specific IL-2 response increased between visit 1 and 3 (FIG. 7B). The response for MTB300 remained the same. Finally, the ATB116-specific TNFα response showed an increase at visit 2 compared to both visit 1 and 3 (FIG. 7C). Again, there was no difference in the response against MTB300. These results indicate that the response against ATB116 changes during treatment for ATB, and is differentially affected depending on the specific cytokine measured.

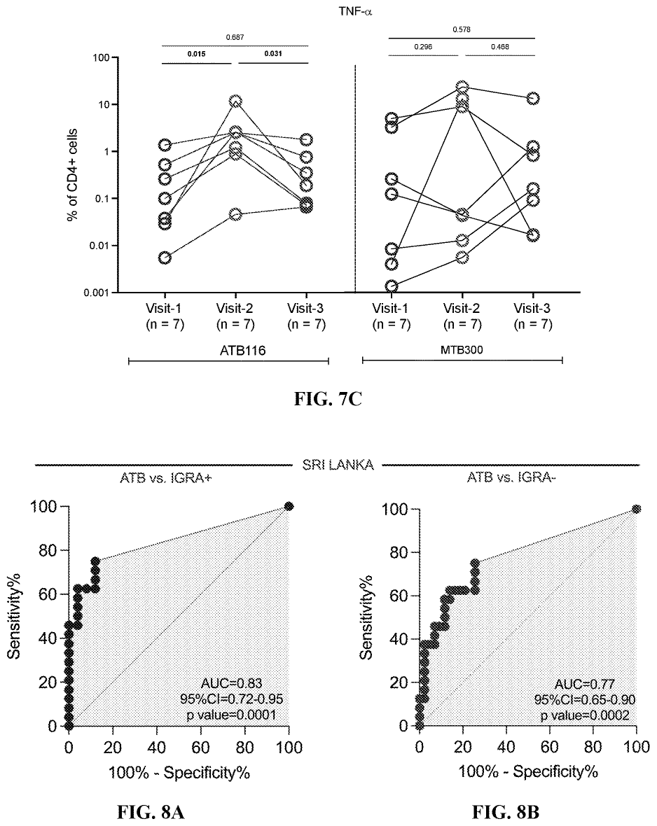

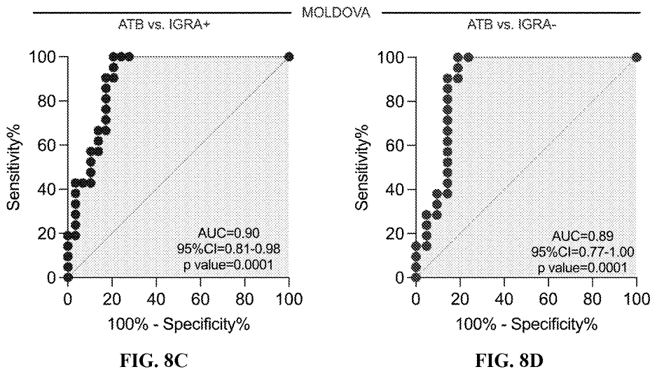

FIGS. 8A to 8D show the diagnostic use of ATB116. Receiver Operating Characteristic (ROC) curves for Sri Lanka (FIG. 8A, FIG. 8B) and Moldova (FIG. 8C, FIG. 8D). ATB vs. IGRA+ (FIG. 8A, FIG. 8C) and ATB vs. IGRA− (FIG. 8B, FIG. 8D). The x-axis shows False positive rate (100%-specificity %) and y-axis shows true positive rate (sensitivity %). Each point on the curve represents a different threshold value used to calculate the true positive rate and false positive rate. Area under curve (AUC) is shown for predictive performance.

### Definitions

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., sgRNA) may be detected by standard PCR or Northern blot methods well known in the art. See, Sambrook et al., 1989 Molecular Cloning: A Laboratory Manual, 18.1-18.88.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may, in embodiments, be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

Proteins and peptides include isolated and purified forms. Proteins and peptides also include those immobilized on a substrate, as well as amino acid sequences, subsequences, portions, homologues, variants, and derivatives immobilized on a substrate.

Proteins and peptides can be included in compositions, for example, a pharmaceutical composition. In particular embodiments, a pharmaceutical composition is suitable for specific or non-specific immunotherapy, or is a vaccine composition.

Isolated nucleic acid (including isolated nucleic acid) encoding the proteins and peptides are also provided. Cells expressing a protein or peptide are further provided. Such cells include eukaryotic and prokaryotic cells, such as mammalian, insect, fungal and bacterial cells.

Methods and uses and medicaments of proteins and peptides of the invention are included. Such methods, uses and medicaments include modulating immune activity of a cell against a pathogen, for example, a bacteria or bacteria.

The term “peptide mimetic” or “peptidomimetic” refers to protein-like chain designed to mimic a peptide or protein. Peptide mimetics may be generated by modifying an existing peptide or by designing a compound that mimic peptides, including peptoids and β-peptides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M) (see, e.g., Creighton, Proteins (1984)).

A “percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “multimer” refers to a complex comprising multiple monomers (e.g., a protein complex) associated by noncovalent bonds. The monomers be substantially identical monomers, or the monomers may be different. In embodiments, the multimer is a dimer, a trimer, a tetramer, or a pentamer.

As used herein, the term “Major Histocompatibility Complex” (MHC) is a generic designation meant to encompass the histocompatibility antigen systems described in different species including the human leucocyte antigens (HLA). Typically, MHC Class I or Class II multimers are well known in the art and include but are not limited to dimers, tetramers, pentamers, hexamers, heptamers and octamers.

As used herein, the term “MHC/peptide multimer” refers to a stable multimeric complex composed of MHC protein(s) subunits loaded with a peptide of the present invention. For example, an MHC/peptide multimer (also called herein MHC/peptide complex) include, but are not limited to, an MHC/peptide dimer, trimer, tetramer, pentamer or higher valency multimer. In humans there are three major different genetic loci that encode MHC class I molecules (the MHC molecules of the human are also designated human leukocyte antigens (HLA)): HLA-A, HLA-B, HLA-C, e.g., HLA-A*01, HLA-A*02, and HLA-A*11 are examples of different MHC class I alleles that can be expressed from these loci. Non-classical human MHC class I molecules such as HLA-E (homolog of mice Qa-lb) and MICA/B molecules are also encompassed by the present invention. In some embodiments, the MHC/peptide multimer is an HLA/peptide multimer selected from the group consisting of HLA-A/peptide multimer, HLA-B/peptide multimer, HLA-C/peptide multimer, HLA-E/peptide multimer, MICA/peptide multimer and MICB/peptide multimer.

In humans there are three major different genetic loci that encode MHC class II molecules: HLA-DR, HLA-DP, and HLA-DQ, each formed of two polypeptides, alpha and beta chains (A and B genes). For example, HLA-DQA1*01, HLA-DRB1*01, and HLA-DRB1*03 are different MHC class II alleles that can be expressed from these loci. It should be further noted that non-classical human MHC class II molecules such as HLA-DM and HL-DOA (homolog in mice is H2-DM and H2-0) are also encompassed by the present invention. In some embodiments, the MHC/peptide multimer is an HLA/peptide multimer selected from the group consisting of HLA-DP/peptide multimer, HLA-DQ/peptide multimer, HLA-DR/peptide multimer, HLA-DM/peptide multimer and HLA-DO/peptide multimer.

An MHC/peptide multimer may be a multimer where the heavy chain of the MHC is biotinylated, which allows combination as a tetramer with streptavidin. MHC-peptide tetramers have increased avidity for the appropriate T cell receptor (TCR) on T lymphocytes. The multimers can also be attached to paramagnetic particles or magnetic beads to facilitate removal of non-specifically bound reporter and cell sorting. Multimer staining does not kill the labelled cells, thus, cell integrity is maintained for further analysis. In some embodiments, the MHC/peptide multimer of the present invention is particularly suitable for isolating and/or identifying a population of CD8+ T cells having specificity for the peptide of the present invention (in a flow cytometry assay).

The peptides or MHC class I or class II multimer as described herein is particularly suitable for detecting T cells specific for one or more peptides of the present invention. The peptide(s) and/or the MHC/multimer complex of the present invention is particularly suitable for diagnosing Mycobacterium infection in a subject. For example, the method comprises obtaining a blood or PBMC sample obtained from the subject with an amount of a least peptide of the present invention and detecting at least one T cell displaying a specificity for the peptide. Another diagnostic method of the present invention involves the use of a peptide of the present invention that is loaded on multimers as described above, so that the isolated CD8+ or CD4+ T cells from the subject are brought into contact with the multimers, at which the binding, activation and/or expansion of the T cells is measured. For example, following the binding to antigen presenting cells, e.g., those having the MHC class I or class II multimer, the number of CD8+ and/or CD4+ cells binding specifically to the HLA-peptide multimer may be quantified by measuring the secretion of lymphokines/cytokines, division of the T cells, or standard flow cytometry methods, such as, for example, using fluorescence activated cell sorting (FACS). The multimers can also be attached to paramagnetic ferrous or magnetic beads to facilitate removal of non-specifically bound reporter and cell sorting.

The MHC class I or class II peptide multimers as described herein can also be used as therapeutic agents. The peptide and/or the MHC class I or class II peptide multimers of the present invention are suitable for treating or preventing a Mycobacterium infection in a subject. The MHC Class I or Class II multimers can be administered in soluble form or loaded on nanoparticles.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The phrase “specifically (or selectively) binds” to an antibody or “specifically (or selectively) immunoreactive with,” when referring to a protein or peptide, refers to a binding reaction that is determinative of the presence of the protein or peptide, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into a Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. The Fc (i.e., fragment crystallizable region) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins.

As used herein, the term “antigen” and the term “epitope” refers to a molecule or substance capable of stimulating an immune response. In one example, epitopes include but are not limited to a polypeptide and a nucleic acid encoding a polypeptide, wherein expression of the nucleic acid into a polypeptide is capable of stimulating an immune response when the polypeptide is processed and presented on a Major Histocompatibility Complex (MHC) molecule. Generally, epitopes include peptides presented on the surface of cells non-covalently bound to the binding groove of Class I or Class II MHC, such that they can interact with T cell receptors and the respective T cell accessory molecules. However, antigens and epitopes also apply when discussing the antigen binding portion of an antibody, wherein the antibody binds to a specific structure of the antigen.

Proteolytic Processing of Antigens. Epitopes that are displayed by MHC on antigen presenting cells are cleavage peptides or products of larger peptide or protein antigen precursors. For MHC I epitopes, protein antigens are often digested by proteasomes resident in the cell. Intracellular proteasomal digestion produces peptide fragments of about 3 to 23 amino acids in length that are then loaded onto the MHC protein. Additional proteolytic activities within the cell, or in the extracellular milieu, can trim and process these fragments further. Processing of MHC Class II epitopes generally occurs via intracellular proteases from the lysosomal/endosomal compartment. The present invention includes, in one embodiment, pre-processed peptides that are attached to the anti-CD40 antibody (or fragment thereof) that directs the peptides against which an enhanced immune response is sought directly to antigen presenting cells.

The present invention includes methods for specifically identifying the epitopes within antigens most likely to lead to the immune response sought for the specific sources of antigen presenting cells and responder T cells.

As used herein, the term “T cell epitope” refers to a specific amino acid that when present in the context of a Major or Minor Histocompatibility Complex provides a reactive site for a T cell receptor. The T-cell epitopes or peptides that stimulate the cellular arm of a subject's immune system are short peptides of about 8-25 amino acids. T-cell epitopes are recognized by T cells from animals that are immune to the antigen of interest. These T-cell epitopes or peptides can be used in assays such as the stimulation of cytokine release or secretion or evaluated by constructing major histocompatibility (MHC) proteins containing or “presenting” the peptide. Such immunogenically active fragments are often identified based on their ability to stimulate lymphocyte proliferation in response to stimulation by various fragments from the antigen of interest.

As used herein, the term “immunological response” refers to an antigen or composition is the development in a subject of a humoral and/or a cellular immune response to an antigen present in the composition of interest. For purposes of the present disclosure, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells. Hence, an immunological response may include one or more of the following effects: the production of antibodies by B-cells; and/or the activation of effector and/or suppressor T-cells and/or gamma-delta T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

As used herein, the term an “immunogenic composition” and “vaccine” refer to a composition that comprises an antigenic molecule where administration of the composition to a subject or patient results in the development in the subject of a humoral and/or a cellular immune response to the antigenic molecule of interest. “Vaccine” refers to a composition that can provide active acquired immunity to and/or therapeutic effect (e.g., treatment) of a particular disease or a pathogen. A vaccine typically contains one or more agents that can induce an immune response in a subject against a pathogen or disease, i.e., a target pathogen or disease. The immunogenic agent stimulates the body's immune system to recognize the agent as a threat or indication of the presence of the target pathogen or disease, thereby inducing immunological memory so that the immune system can more easily recognize and destroy any of the pathogen on subsequent exposure. Vaccines can be prophylactic (e.g., preventing or ameliorating the effects of a future infection by any natural or pathogen) or therapeutic (e.g., reducing symptoms or aberrant conditions associated with infection). The administration of vaccines is referred to vaccination.

In some examples, a vaccine composition can provide nucleic acid, e.g., mRNA that encodes antigenic molecules (e.g., peptides) to a subject. The nucleic acid that is delivered via the vaccine composition in the subject can be expressed into antigenic molecules and allow the subject to acquire immunity against the antigenic molecules. In the context of the vaccination against infectious disease, the vaccine composition can provide mRNA encoding antigenic molecules that are associated with a certain pathogen, e.g., one or more peptides that are known to be expressed in the pathogen (e.g., pathogenic bacterium or bacteria).

The present invention provides nucleic acid molecules, specifically polynucleotides, primary constructs and/or mRNA that encode one or more polynucleotides that express one or more peptides or proteins, comprising, consisting of, or consisting essentially of an amino acid sequence selected from any one of those sequences set forth in Tables 1-5 (SEQ ID NOS:1-718), or a subsequence, portion, homologue, variant or derivative thereof for use in immune modulation. The term “nucleic acid” refers to any compound and/or substance that comprise a polymer of nucleotides, referred to herein as polynucleotides. Exemplary nucleic acids or polynucleotides of the invention include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs), including diastereomers of LNAs, functionalized LNAs, or hybrids thereof.

One method of immune modulation of the present invention includes direct or indirect gene transfer, i.e., local application of a preparation containing the one or more polynucleotides (DNA, RNA, mRNA, etc.) that expresses the one or more peptides or proteins, comprising, consisting of, or consisting essentially of an amino acid sequence selected from any one of those sequences set forth in Tables 1-5 (SEQ ID NOS:1-718), or a subsequence, portion, homologue, variant or derivative thereof. A variety of well-known vectors can be used to deliver to cells the one or more polynucleotides or the peptides or proteins expressed by the polynucleotides, including but not limited to adenobacterial vectors and adeno-associated vectors. In addition, naked DNA, liposome delivery methods, or other novel vectors developed to deliver the polynucleotides to cells can also be beneficial. Any of a variety of promoters can be used to drive peptide or protein expression, including but not limited to endogenous promoters, constitutive promoters (e.g., cytomegalobacteria, adenobacteria, or SV40), inducible promoters (e.g., a cytokine promoter such as the interleukin-1, tumor necrosis factor-alpha, or interleukin-6 promoter), and tissue specific promoters to express the immunogenic peptides or proteins of the present invention.

The immunization may include adenobacteria, adeno-associated bacteria, herpes bacteria, vaccinia bacteria, retrobacteriaes, or other bacterial vectors with the appropriate tropism for cells likely to present the antigenic peptide(s) or protein(s) may be used as a gene transfer delivery system for a therapeutic peptide(s) or protein(s), comprising, consisting of, or consisting essentially of an amino acid sequence selected from any one of those sequences set forth in Tables 1-5 (SEQ ID NOS:1-718), or a subsequence, portion, homologue, variant or derivative thereof, gene expression construct. Bacterial vectors which do not require that the target cell be actively dividing, such as adenobacterial and adeno-associated vectors, are particularly useful when the cells are accumulating, but not proliferative. Numerous vectors useful for this purpose are generally known (Miller, Human Gene Therapy 15-14, 1990; Friedman, Science 244:1275-1281, 1989; Eglitis and Anderson, BioTechniques 6:608-614, 1988; Tolstoshev and Anderson, Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; and Miller and Rosman, Bio Techniques 7:980-990, 1989; Le Gal La Salle et al., Science 259:988-990, 1993; and Johnson, Chest 107:77S-83S, 1995). Retrobacterial vectors are particularly well developed and have been used in clinical settings (Rosenberg et al., N. Engl. J. Med 323:370, 1990; Anderson et al., U.S. Pat. No. 5,399,346).

The immunization may also include inserting the one or more polynucleotides (DNA, RNA, mRNA, etc.) that express the one or more peptides or proteins, comprising, consisting of, or consisting essentially of an amino acid sequence selected from any one of those sequences set forth in Tables 1-5 (SEQ ID NOS:1-718), or a subsequence, portion, homologue, variant or derivative thereof into the bacterial vector, along with another gene which encodes the ligand for a receptor on a specific target cell, for example, such that the vector is now target specific. Bacterial vectors can be made target specific by attaching, for example, a sugar, a glycolipid, or a protein. Targeting can also be accomplished by using an antibody to target the bacterial vector. Those of skill in the art will know of, or can readily ascertain without undue experimentation, specific polynucleotide sequences which can be inserted into the bacterial genome or attached to a bacterial envelope to allow target specific delivery of the bacterial vector containing the gene.

Since recombinant bacteriaes are defective, they require assistance in order to produce infectious vector particles. This assistance can be provided, for example, by using helper cell lines that contain plasmids encoding all of the structural genes of the bacteria under the control of regulatory sequences within the bacterial genome. These plasmids are missing a nucleotide sequence which enables the packaging mechanism to recognize a polynucleotide transcript for encapsidation. These cell lines produce empty virions, since no genome is packaged. If a bacterial vector is introduced into such cells in which the packaging signal is intact, but the structural genes are replaced by other genes of interest, the vector can be packaged and vector virion produced.

Bacterial or non-bacterial approaches may also be employed for the introduction of one or more therapeutic polynucleotides that express the one or more peptides or proteins, comprising, consisting of, or consisting essentially of an amino acid sequence selected from any one of those sequences set forth in Tables 1-5 (SEQ ID NOS:1-718), or a subsequence, portion, homologue, variant or derivative thereof, into polynucleotide-encoding polynucleotide into antigen presenting cells. The polynucleotides may be DNA, RNA, mRNA that directly encode the one or more peptides or proteins of the present invention, or may be introduced as part of an expression vector.

Another example of an immunization includes colloidal dispersion systems that include macromolecule complexes, nanocapsules, microspheres, beads, and lipid-based systems including oil-in-water emulsions, micelles, mixed micelles, and liposomes and the one or more polynucleotides that express the one or more peptides or proteins, comprising, consisting of, or consisting essentially of an amino acid sequence selected from any one of those sequences set forth in Tables 1-5 (SEQ ID NOS:1-718), or a subsequence, portion, homologue, variant or derivative thereof. One non-limiting example of a colloidal system for use with the present invention is a liposome. Liposomes are artificial membrane vesicles which are useful as delivery vehicles in vitro and in vivo. It has been shown that large unilamellar vesicles (LUV), which range in size from 0.2-4.0 micrometers that can encapsulate a substantial percentage of an aqueous buffer containing large macromolecules. RNA, DNA and intact virions can be encapsulated within the aqueous interior and be delivered to cells in a biologically active form (Fraley, et al., Trends Biochem. Sci., 6:77, 1981). In addition to mammalian cells, liposomes have been used for delivery of polynucleotides in plant, yeast and bacterial cells. In order for a liposome to be an efficient gene transfer vehicle, the following characteristics should be present: (Zakut and Givol, supra) encapsulation of the genes of interest at high efficiency while not compromising their biological activity; (Fearnhead, et al., supra) preferential and substantial binding to a target cell in comparison to non-target cells; (Korsmeyer, S. J., supra) delivery of the aqueous contents of the vesicle to the target cell cytoplasm at high efficiency; and (Kinoshita, et al., supra) accurate and effective expression of genetic information (Mannino, et al., Bio Techniques, 6:682, 1988).

The composition for immunizing the subject or patient may, in certain embodiments comprise a combination of phospholipid, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticuloendothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization, specifically, cells that can become infected with a Mycobacterium or interact with the proteins, peptides, and/or gene products of a Mycobacterium, e.g., immune cells.

For any of the above approaches, the immune modulating polynucleotide construct, composition, or formulation is preferably applied to a site that will enhance the immune response. For example, the immunization may be intramuscular, intraperitoneal, enteral, parenteral, intranasal, intrapulmonary, or subcutaneous. In the gene delivery constructs of the instant invention, polynucleotide expression is directed from any suitable promoter (e.g., the human cytomegalobacteria, simian bacteria 40, actin or adenobacteria constitutive promoters; or the cytokine or metalloprotease promoters for activated synoviocyte specific expression).

In one example of the immune modifying peptide(s) or protein(s) include polynucleotides, constructs and/or mRNAs that express the one or more polynucleotides that express the one or more peptides or proteins, comprising, consisting of, or consisting essentially of an amino acid sequence selected from any one of those sequences set forth in Tables 1-5 (SEQ ID NOS:1-718), or a subsequence, portion, homologue, variant or derivative thereof, that are designed to improve one or more of the stability and/or clearance in tissues, uptake and/or kinetics, cellular access by the peptide(s) or protein(s), translational, mRNA half-life, translation efficiency, immune evasion, protein production capacity, accessibility to circulation, peptide(s) or protein(s) half-life and/or presentation in the context of MHC on antigen presenting cells.

The present invention contemplates immunization for use in both active and passive immunization embodiments. Immunogenic compositions, proposed to be suitable for use as a vaccine, may be prepared most readily directly from immunogenic peptides, proteins, monomers, multimers and/or peptide-MHC complexes prepared in a manner disclosed herein. The antigenic material is generally processed to remove undesired contaminants, such as, small molecular weight molecules, incomplete proteins, or when manufactured in plant cells, plant components such as cell walls, plant proteins, and the like. Often, these immunizations are lyophilized for ease of transport and/or to increase shelf-life and can then be more readily dissolved in a desired vehicle, such as saline.

The preparation of immunizations (also referred to as vaccines) that contain the immunogenic proteins of the present invention as active ingredients is generally well understood in the art, as exemplified by U.S. Pat. Nos. 4,608,251; 4,601,903; 4,599,231; 4,599,230; 4,596,792; and 4,578,770, all incorporated herein by reference. Typically, such immunizations are prepared as injectables. The immunizations can be a liquid solution or suspension but may also be provided in a solid form suitable for solution in, or suspension in, liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, buffers, or the like and combinations thereof. In addition, if desired, the immunization may contain minor amounts of auxiliary substances such as wetting or emulsifying agents, pH buffering agents, or adjuvants which enhance the effectiveness of the vaccines.

The immunization is/are administered in a manner compatible with the dosage formulation, and in such amount as will be therapeutically effective and immunogenic. The quantity to be administered depends on the subject to be treated, including, e.g., the capacity of the individual's immune system to synthesize antibodies, and the degree of protection desired. Precise amounts of active ingredient required to be administered depend on the judgment of the practitioner. However, suitable dosage ranges are of the order of several hundred micrograms active ingredient per vaccination. Suitable regimes for initial administration and booster shots are also variable but are typified by an initial administration followed by subsequent inoculations or other administrations.

The manner of application of the immunization may be varied widely. Any of the conventional methods for administration of a vaccine are applicable. These are believed to also include oral application on a solid physiologically acceptable base or in a physiologically acceptable dispersion, parenterally, by injection or the like. The dosage of the vaccine will depend on the route of administration and will vary according to the size of the host.

Various methods of achieving adjuvant effect for the vaccine includes use of agents such as aluminum hydroxide or phosphate (alum), commonly used as 0.05 to 0.1 percent solution in phosphate buffered saline, admixture with synthetic polymers of sugars (Carbopol) used as 0.25 percent solution, aggregation of the protein in the vaccine by heat treatment with temperatures ranging between 70° to 101° C. for 30 second to 2-minute periods respectively. Aggregation by reactivating with pepsin treated (Fab) antibodies to albumin, mixture with bacterial cells such as C. parvum or endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be employed.

In many instances, it will be desirable to have multiple administrations of the vaccine, usually not exceeding six to ten immunizations, more usually not exceeding four immunizations and preferably one or more, usually at least about three immunizations. The immunizations will normally be at from two to twelve-week intervals, more usually from three to five-week intervals. Periodic boosters at intervals of 1-5 years, usually three years, will be desirable to maintain protective levels of the antibodies. The course of the immunization may be followed by assays for antibodies for the supernatant antigens. The assays may be performed by labeling with conventional labels, such as radionuclides, enzymes, fluorescent agents, and the like. These techniques are well known and may be found in a wide variety of patents, such as Hudson and Cranage, Vaccine Protocols, 2003 Humana Press, relevant portions incorporated herein by reference.

Techniques and compositions for making useful dosage forms using the present invention are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2007; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000, and updates thereto; Martindale, The Extra Pharmacopoeia, Thirty-Second Edition (The Pharmaceutical Press, London, 1999); all of which are incorporated by reference, and the like, relevant portions incorporated herein by reference.

Many suitable expression systems are commercially available, including, for example, the following: baculobacteria expression (Reilly, P. R., et al., BACULOBACTERIA EXPRESSION VECTORS: A LABORATORY MANUAL (1992); Beames, et al., Biotechniques 11:378 (1991); Pharmingen; Clontech, Palo Alto, Calif)), vaccinia expression systems (Earl, P. L., et al., “Expression of proteins in mammalian cells using vaccinia” In Current Protocols in Molecular Biology (F. M. Ausubel, et al. Eds.), Greene Publishing Associates & Wiley Interscience, New York (1991); Moss, B., et al., U.S. Pat. No. 5,135,855, issued Aug. 4, 1992), expression in bacteria (Ausubel, F. M., et al., CURRENT PROTOCOLS IN MOLECULAR BIOLOGY, John Wiley and Sons, Inc., Media Pa.; Clontech), expression in yeast (Rosenberg, S. and Tekamp-Olson, P., U.S. Pat. No. RE35,749, issued, Mar. 17, 1998, herein incorporated by reference; Shuster, J. R., U.S. Pat. No. 5,629,203, issued May 13, 1997, herein incorporated by reference; Gellissen, G., et al., Antonie Van Leeuwenhoek, 62(1-2):79-93 (1992); Romanos, M. A., et al., Yeast 8(6):423-488 (1992); Goeddel, D. V., Methods in Enzymology 185 (1990); Guthrie, C., and G. R. Fink, Methods in Enzymology 194 (1991)), expression in mammalian cells (Clontech; Gibco-BRL, Ground Island, N.Y.; e.g., Chinese hamster ovary (CHO) cell lines (Haynes, J., et al., Nuc. Acid. Res. 11:687-706 (1983); 1983, Lau, Y. F., et al., Mol. Cell. Biol. 4:1469-1475 (1984); Kaufman, R. J., “Selection and coamplification ofheterologous genes in mammalian cells,” in Methods in Enzymology, vol. 185, pp 537-566. Academic Press, Inc., San Diego Calif. (1991)), and expression in plant cells (plant cloning vectors, Clontech Laboratories, Inc., Palo-Alto, Calif, and Pharmacia LKB Biotechnology, Inc., Pistcataway, N.J.; Hood, E., et al., J. Bacteriol. 168:1291-1301 (1986); Nagel, R., et al., FEMS Microbiol. Lett. 67:325 (1990); An, et al., “Binary Vectors”, and others in Plant Molecular Biology Manual A3:1-19 (1988); Miki, B. L. A., et al., pp. 249-265, and others in Plant DNA Infectious Agents (Hohn, T., et al., eds.) Springer-Verlag, Wien, Austria, (1987); Plant Molecular Biology: Essential Techniques, P. G. Jones and J. M. Sutton, New York, J. Wiley, 1997; Miglani, Gurbachan Dictionary of Plant Genetics and Molecular Biology, New York, Food Products Press, 1998; Henry, R. J., Practical Applications of Plant Molecular Biology, New York, Chapman & Hall, 1997), relevant portion incorporated herein by reference.

As used herein, the term “effective amount” or “effective dose” refers to that amount of the peptide or protein T cell epitopes of the invention sufficient to induce immunity, to prevent and/or ameliorate an infection or to reduce at least one symptom of an infection and/or to enhance the efficacy of another dose of peptide or protein T cell epitopes. An effective dose may refer to the amount of peptide or protein T cell epitopes sufficient to delay or minimize the onset of an infection. An effective dose may also refer to the amount of peptide or protein T cell epitopes that provides a therapeutic benefit in the treatment or management of an infection. Further, an effective dose is the amount with respect to peptide or protein T cell epitopes of the invention alone, or in combination with other therapies, that provides a therapeutic benefit in the treatment or management of an infection. An effective dose may also be the amount sufficient to enhance a subject's (e.g., a human's) own immune response against a subsequent exposure to an infectious agent. Levels of immunity can be monitored, e.g., by measuring amounts of neutralizing secretory and/or serum antibodies, e.g., by plaque neutralization, complement fixation, enzyme-linked immunosorbent, or microneutralization assay. In the case of a vaccine, an “effective dose” is one that prevents disease and/or reduces the severity of symptoms. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms, in this case, an infectious disease, and more particularly, a Mycobacterium infection. The full prophylactic effect does not necessarily occur by administration of one dose, and may occur only after administration of a series of doses. Thus, a prophylactically effective amount may be administered in one or more administrations. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins), relevant portions incorporated herein by reference.

As used herein, the term “immune stimulator” refers to a compound that enhances an immune response via the body's own chemical messengers (cytokines). These molecules comprise various cytokines, lymphokines and chemokines with immunostimulatory, immunopotentiating, and pro-inflammatory activities, such as interferons, interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immune stimulator molecules can be administered in the same formulation as peptide or protein T cell epitopes s of the invention, or can be administered separately. Either the protein or an expression vector encoding the protein can be administered to produce an immunostimulatory effect.

As used herein, in certain embodiments, the term “protective immune response” or “protective response” refers to an immune response mediated by antibodies against an infectious agent, which is exhibited by a vertebrate (e.g., a human), which prevents or ameliorates an infection or reduces at least one symptom thereof. Peptide and protein T cell epitopes of the invention can stimulate the production of antibodies that, for example, neutralize infectious agents, blocks infectious agents from entering cells, blocks replication of said infectious agents, and/or protect host cells from infection and destruction. In other embodiments, the term can also refer to an immune response that is mediated by T-lymphocytes and/or other white blood cells against an infectious agent, exhibited by a vertebrate (e.g., a human), that prevents or ameliorates flavibacteria infection or reduces at least one symptom thereof. Peptide and protein T cell epitopes of the invention can stimulate the T cell responses that, for example, neutralize infectious agents, kill bacteria infected cells, blocks infectious agents from entering cells, blocks replication of said infectious agents, and/or protect host cells from infection and destruction.

The terms “biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

As used herein, a “cell” refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

As used herein, the term “contacting” is used in accordance with its plain ordinary meaning and refers to the process of allowing at least two distinct species to become sufficiently proximal to react, interact or physically touch. It should be appreciated, however, the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be, for example, an amino acid sequence, protein, or peptide as provided herein and an immune cell, such as a T cell.

As used herein, a “control” sample or value refers to a sample that serves as a reference, usually a known reference, for comparison to a test sample. For example, a test sample can betaken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The term “modulator” refers to a composition that increases or decreases the level of a target molecule or the function of a target molecule or the physical state of the target of the molecule relative to the absence of the modulator.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The terms “associated” or “associated with” in the context of a substance or substance activity or function associated with a disease (e.g. a protein associated disease, a cancer (e.g., cancer, inflammatory disease, autoimmune disease, or infectious disease)) means that the disease (e.g. cancer, inflammatory disease, autoimmune disease, or infectious disease) is caused by (in whole or in part), or a symptom of the disease is caused by (in whole or in part) the substance or substance activity or function. As used herein, what is described as being associated with a disease, if a causative agent, could be a target for treatment of the disease.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The terms “subject” or “subject in need thereof” refers to a living organism who is at risk of or prone to having a disease or condition, or who is suffering from a disease or condition that can be treated by administration of a composition or pharmaceutical composition as provided herein. Non-limiting examples include humans and other primates, but also includes non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The system described above is intended for use in any of the above vertebrate species, since the immune systems of all of these vertebrates operate similarly.

The terms “disease” or “condition” refer to a state of being or health status of a patient or subject capable of being treated with a compound, pharmaceutical composition, or method provided herein. In embodiments, a patient or subject is human. In embodiments, the disease is Mycobacterium infection. In certain alternative embodiments, the disease is MTB infection. In still other embodiments, the disease is whooping cough.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated or the disorder resulting from bacterial infection. Also, a therapeutic benefit is achieved with the eradication or amelioration of one or more of the physiological symptoms associated with bacterial infection or the underlying disorder such that an improvement is observed in the patient, notwithstanding that the patient may still be afflicted with the underlying disorder or may still be infected. For prophylactic benefit, the compositions may be administered to a patient at risk of bacterial infection, of developing a particular disease, or to a patient reporting one or more of the physiological symptoms of a disease, even though a diagnosis of this disease may not have been made. Treatment includes preventing the infection or disease, that is, causing the clinical symptoms of the disease not to develop by administration of a protective composition prior to infection or the induction of the disease; suppressing the disease, that is, causing the clinical symptoms of the disease or infection not to develop by administration of a protective composition after the inductive event or infection but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance. “Treatment” can also refer to any of (i) the prevention of infection or reinfection, as in a traditional vaccine, (ii) the reduction or elimination of symptoms, and (iii) the substantial or complete elimination of the pathogen in question. Treatment may be affected prophylactically (prior to infection) or therapeutically (following infection).

In addition, in certain embodiments, “treatment,” “treat,” or “treating” refers to a method of reducing the effects of one or more symptoms of infection with a Mycobacterium. Thus, in the disclosed method, treatment can refer to a 10%, 20%, 30%, 40%, 50%, 60%, 70%, 80%, 90%, or 100% reduction in the severity of an established infection, disease, condition, or symptom of the infection, disease or condition. For example, a method for treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition and/or complete prevention of infection. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

As used herein the terms “diagnose” or “diagnosing” refers to recognition of an infection, disease or condition by signs and symptoms. Diagnosing can refer to determination of whether a subject has an infection or disease, including distinguishing between latent or active infection. Diagnosis may refer to determination of the type of disease or condition a subject has or the type of bacteria the subject is infected with.

Diagnostic agents provided herein include any such agent, which are well-known in the relevant art. Among imaging agents are fluorescent and luminescent substances, including, but not limited to, a variety of organic or inorganic small molecules commonly referred to as “dyes,” “labels,” or “indicators.” Examples include fluorescein, rhodamine, acridine dyes, Alexa dyes, and cyanine dyes. Enzymes that may be used as imaging agents in accordance with the embodiments of the disclosure include, but are not limited to, horseradish peroxidase, alkaline phosphatase, acid phosphatase, glucose oxidase, β-galactosidase, β-glucoronidase or β-lactamase. Such enzymes may be used in combination with a chromogen, a fluorogenic compound or a luminogenic compound to generate a detectable signal.

The peptide(s) or protein(s) of the present invention can also be used in binding assays including, but are not limited to, immunoassays such as competitive and non-competitive assay systems using techniques such as western blots, radioimmunoassays, ELISA (enzyme linked immunosorbent assay), “sandwich” immunoassays, Meso Scale Discovery (MSD, Gaithersburg, Md.), immunoprecipitation assays, ELISPOT, precipitin reactions, gel diffusion precipitin reactions, immunodiffusion assays, agglutination assays, complement-fixation assays, immunoradiometric assays, fluorescent immunoassays, and protein A immunoassays. Such assays are routine and well known in the art (see, e.g., Ausubel et al., eds, 1994, Current Protocols in Molecular Biology, Vol. 1, John Wiley & Sons, Inc., New York, relevant portions incorporated herein by reference).

Radioactive substances that may be used as imaging agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹T, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²P, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Th, Dy, Ho, Er, Tm, Yb and Lu.

When the imaging agent is a radioactive metal or paramagnetic ion, the agent may be reacted with another long-tailed reagent having a long tail with one or more chelating groups attached to the long tail for binding to these ions. The long tail may be a polymer such as a polylysine, polysaccharide, or other derivatized or derivatizable chain having pendant groups to which the metals or ions may be added for binding. Examples of chelating groups that may be used according to the disclosure include, but are not limited to, ethylenediaminetetraacetic acid (EDTA), diethylenetriaminepentaacetic acid (DTPA), DOTA, NOTA, NETA, TETA, porphyrins, polyamines, crown ethers, bis-thiosemicarbazones, polyoximes, and like groups.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

Formulations suitable for oral administration can consist of (a) liquid solutions, such as an effective amount of the antibodies provided herein suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

The term “adjuvant” refers to a compound that when administered in conjunction with the compositions provided herein including embodiments thereof, augments the composition's immune response. Generally, adjuvants are non-toxic, have high-purity, are degradable, and are stable.

Adjuvants can augment an immune response by several mechanisms including lymphocyte recruitment, stimulation of B and/or T cells, and stimulation of macrophages. The adjuvant increases the titer of induced antibodies and/or the binding affinity of induced antibodies relative to the situation if the immunogen were used alone. A variety of adjuvants can be used in combination with the agents provided herein including embodiments thereof, to elicit an immune response. Preferred adjuvants augment the intrinsic response to an immunogen without causing conformational changes in the immunogen that affect the qualitative form of the response. Preferred adjuvants include aluminum hydroxide and aluminum phosphate, 3 De-O-acylated monophosphoryl lipid A (MPL™) (see GB 2220211 (RIBI ImmunoChem Research Inc., Hamilton, Montana, now part of Corixa). Stimulon™ QS-21 is atriterpene glycoside or saponin isolated from the bark of the Quillaja Saponaria Molina tree found in South America (see Kensil et al., in Vaccine Design: The Subunit and Adjuvant Approach (eds. Powell & Newman, Plenum Press, NY, 1995); U.S. Pat. No. 5,057,540), (Aquila BioPharmaceuticals, Framingham, MA). Other adjuvants are oil in water emulsions (such as squalene or peanut oil), optionally in combination with immune stimulants, such as monophosphoryl lipid A (see Stoute et al., N. Engl. J. Med. 336, 86-91 (1997)), pluronic polymers, and killed mycobacteria. Another adjuvant is CpG (WO 98/40100). Adjuvants can be administered as a component of a therapeutic composition with an active agent or can be administered separately, before, concurrently with, or after administration of the therapeutic agent.

Other adjuvants contemplated for the invention are saponin adjuvants, such as Stimulon™ (QS-21, Aquila, Framingham, MA) or particles generated therefrom such as ISCOMs (immunostimulating complexes) and ISCOMATRIX. Other adjuvants include RC-529, GM-CSF and Complete Freund's Adjuvant (CFA) and Incomplete Freund's Adjuvant (IFA). Other adjuvants include cytokines, such as interleukins (e.g., IL-1α and R peptides, IL-2, IL-4, IL-6, IL-12, IL-13, and IL-15), macrophage colony stimulating factor (M-CSF), granulocyte-macrophage colony stimulating factor (GM-CSF), tumor necrosis factor (TNF), chemokines, such as MIP1α and β and RANTES. Another class of adjuvants is glycolipid analogues including N-glycosylamides, N-glycosylureas and N-glycosylcarbamates, each of which is substituted in the sugar residue by an amino acid, as immuno-modulators or adjuvants (see U.S. Pat. No. 4,855,283). Heat shock proteins, e.g., HSP70 and HSP90, may also be used as adjuvants.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

The combined administration contemplates co-administration, using separate formulations or a single pharmaceutical formulation, and consecutive administration in either order, wherein preferably there is a time period while both (or all) active agents simultaneously exert their biological activities.

Effective doses of the compositions provided herein vary depending upon many different factors, including means of administration, target site, physiological state of the patient, whether the patient is human or an animal, other medications administered, and whether treatment is prophylactic or therapeutic. However, a person of ordinary skill in the art would immediately recognize appropriate and/or equivalent doses looking at dosages of approved compositions for treating and preventing cancer for guidance.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration. As used herein, the terms “pharmaceutically acceptable” or “pharmacologically acceptable” refer to a material which is not biologically or otherwise undesirable, i.e., the material may be administered to an individual in a formulation or composition without causing any unacceptable biological effects or interacting in a deleterious manner with any of the components of the composition in which it is contained.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances, and the like, that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

Materials and Methods: PBMC isolation and thawing. PBMCs were obtained by density gradient centrifugation (Ficoll-Hypaque, Amersham Biosciences) from leukapheresis or whole blood samples, according to the manufacturer's instructions. The PBMC processing at the site in the Republic of Moldova used SepMate tubes (StemCell). Cells were resuspended in FBS (Gemini Bio-Products) containing 10% DMSO (v/v, Sigma) and cryopreserved in liquid nitrogen.

Cryopreserved PBMC were quickly thawed by incubating each cryovial at 37° C. for 2 min, and cells transferred to cold medium (RPMI 1640 with L-glutamin and 25 mM HEPES; Omega Scientific), supplemented with 5% human AB serum (GemCell), 1% penicillin streptomycin (Life Technologies), 1% glutamax (Life Technologies) and 20 U/ml benzonase nuclease (MilliporeSigma). Cells were centrifuged and resuspended in complete RPMI medium to determine cell concentration and viability using trypan blue.

Peptide screening and peptide pool preparation. The present study screened a total of 21,220 peptides. These peptides include the same library that was screened in IGRA+ participants of 20,610 Mtb peptides (2 to 10 per ORF, average 5), including 1,660 variants not totally conserved amongst the selected Mtb genomes: five complete Mtb genomes (CDC1551, F11, H37Ra, H37Rv and KZN 1435) and sixteen draft assemblies (22). Along with these peptides, 610 peptides were also selected, which include 93 peptides that are not found in Mtb but present in the Mycobacterium bovis BCG strains Mexico, Tokyo 172, and Pasteur 1173P2, and 517 peptides that are 15-mers overlapping by ten amino acids spanning the entire sequence of 12 TB vaccine candidate and IGRA antigens. The vaccine candidates included ID93: GLA-SE (Rv3619c, Rv3620c, Rv1813, and Rv2608), H1:IC31 (ESAT-6 and Ag85B), H4:IC31 (Ag85B, TB10.4), H56:IC31 (Ag85B, ESAT-6, and Rv2660c), M72/AS01E (Mtb32A, PPE18), and three candidates with Ag85A alone (Ad5 Ag85A, ChAdOx1-85A/MVA85A, and MVA85A). The IGRA antigens include ESAT-6, which is also a vaccine candidate antigen, and CFP10.

The peptides were synthesized as crude material on a small (1 mg) scale by Mimotopes (Australia). The peptides were solubilized using DMSO at a concentration of 20 mg/ml. The peptides were pooled into peptide pools. The previous peptide library was pooled into 1036 peptide pools of about 20 peptides each (average 19.9±0.5). The 93 non-Mtb peptides were pooled into 5 peptide pools. The 15-mer peptides overlapping by 10 amino acids spanning the TB vaccine candidate and IGRA antigens were pooled into 26 peptide pools. Thus, from the 21,220 total peptides, a total of 1,067 peptide pools were made.

Individual peptides were mixed in equal amounts after being dissolved in DMSO for the megapools (PC85, PC71, ATB116, and MTB300), as described previously (37). Each peptide pool was then placed in a lyophilizing flask and subjected to lyophilization for 24 hours. The resulting semi-solid product was dissolved in water, frozen, and lyophilized again until only solid product remained. This process was repeated several times until only solid product remained after lyophilization. Finally, the peptide pool was re-suspended in DMSO at a higher concentration per peptide (0.7 mg/ml per peptide) than before lyophilization, to reduce the concentration of DMSO in the assays.

Ex vivo IFNγ and IL-17 fluorospot assay. IFNγ and IL-17 production was measured by a Fluorospot assay with all antibodies and reagents from Mabtech (Nacka Strand, Sweden). Plates were coated overnight at 4° C. with an antibody mixture containing mouse anti-human IFNγ (clone 1-D1K) and mouse anti-human IL-17 (clone MT44.6). Briefly, 2×10⁵ PBMCs were added to each well of pre-coated Immobilion-FL PVDF 96-well plate in the presence of peptide pools at a concentration of 2 μg/ml, individual peptides at 5 μg/ml, PHA at 10 μg/ml (positive control) and media containing DMSO (amount corresponding to percent DMSO in the pools/peptides, as a negative control). Plates were incubated at 37° C. in a humidified CO₂ incubator for 20-24 hours. All conditions were tested in triplicate, except the negative control, which was tested in six individual wells. After incubation, plates were developed according to the manufacturer's instructions. Briefly, cells were removed and wells were washed with PBS/0.05% Tween 20 using an automated plate washer. After washing, an antibody mixture containing anti-IFNγ (7-B6-1-FS-BAM) and anti-IL-17 (MT504-WASP) prepared in PBS with 0.1% BSA was added to each well and plates were incubated for 2 hours at room temperature. The plates were again washed and incubated with diluted fluorophore-conjugated anti-BAM-490 and anti-WASP-640 antibody for 1 hour at room temperature. Finally the plates were washed and incubated with a fluorescence enhancer for 15 min, blotted dry and fluorescent spots were counted by computer assisted image analysis (IRIS Fluorospot reader, Mabtech, Sweden).

Each pool or peptide was considered positive compared to the background that had an equivalent amount of DMSO based on the following criteria: (i) 20 or more spot-forming cells (SFC) per 10⁶ PBMC after background subtraction, (ii) a greater than 2-fold increase compared to the background, and (iii) p<0.05 by student's t-test or Poisson distribution test when comparing the peptide or pool triplicates with the negative control. The response frequency was calculated by dividing the number of participants responding by the no. of participants tested. The magnitude of response (total SFC) was calculated by summation of SFC from responding participants.

Intracellular cytokine staining assay. PBMC at 1×10⁶ per condition were stimulated with peptide pools (2 μg/ml) for 18-20 h in complete RPMI medium at 37° C. with 5% CO₂. PBMCs incubated with DMSO at the percentage corresponding to the amount in the peptide pools were used as a negative control to assess nonspecific or background cytokine production, and anti-CD3/CD28 (1 μg/ml; OKT3 and CD28.2) stimulation was used as a positive control. For Chemokine receptor staining, antibodies were added during the stimulation. After 18 hours, 2.5 μg/ml each of BFA and monensin was added for an additional 5 h at 37° C. Cells were then harvested and incubated in a blocking buffer containing 10% FBS and 1 μg/mL Human Fc block (BD Biosciences, USA) for 20 minutes at 4° C. Next, cells were stained using fixable live/dead stain for 20 minutes at room temperature and then stained with surface-expressed antibodies diluted in FACS buffer and 1× Brilliant Stain Buffer (BD Biosciences, USA) for 20 minutes at room temperature. Cells were permeabilized and fixed for intracellular cytokine staining using cytoperm fixation buffer (Biolegend) for 20 minutes at room temperature. After incubation, cells were stained for cytokines (IFNγ, IL-2 and TNFα) for 20 minutes at room temperature. Samples were acquired on a ZE5 cell analyzer (BioRad). Frequencies of CD4 or CD8 T cells responding to each peptide pool were quantified by determining the total number of gated CD4 or CD8 and cytokine-producing cells and background values subtracted (as determined from the negative control) using FlowJo X Software. Combinations of cytokine-producing cells were determined using Boolean gating. The lower limit of detection for the frequency of cytokine-producing CD4 T cells after background subtraction was set to 0.0001%.

Statistical analysis. Statistical analyses were performed using GraphPad Prism software (GraphPad Software, Inc., San Diego, CA, USA, version 9.2). Data is shown as median with interquartile range. Non-parametric test was applied after checking for normality. A two-tailed Mann-Whitney U test was used for comparison between two groups. Wilcoxon signed-rank tests were used for the comparison of cytokine-producing cells in longitudinal samples from ATB patients. p value≤0.05 was considered significant.

The present invention describes methods utilizing and compositions comprising or expressing T cell epitopes, T cell epitope-containing peptides, and T cell epitope-containing proteins associated with binding to a subset of the naturally occurring MHC Class II and/or MHC Class I molecules within the human population. Compositions comprising or expressing one or more of the disclosed peptides (e.g., the amino acid sequences set forth in any one of Tables 1-5 (SEQ ID NOS:1-718)) or polynucleotides encoding the same, covering different HLA Class II and/or MHC Class I alleles, capable of generating a treatment acting broadly on a population level are disclosed herein. As the antigen repertoire of MHC Class I and MHC Class II alleles vanes from one individual to another and from one ethnic population to another, it is challenging to provide vaccines or peptide or epitopes-based immunotherapies that can be offered to subjects of any geographic region in the world or provide sufficient protection against infection across a wide segment of the populations unless numerous epitopes or peptides are included (e.g., in a vaccine). Taking into consideration the need for a single vaccine formulation that can provide protection across populations, if it desirable to provide a treatment containing or expressing proteins, peptides or epitopes that will provide protection against infection amongst the majority of the worldwide population. Also, taking into consideration the enormous costs and risks in the clinical development of new treatments and the increasing demands from regulatory bodies to meet high standards for toxicity testing, dose justification, safety and efficacy trials, it is desirable to provide treatments containing or expressing as few peptides as possible, but at the same time to be able to treat the majority of subjects in a worldwide population with a single immunotherapy. Such a product should comprise as a first requirement an expression or inclusion of combination of epitopes or peptides that are able to bind the worldwide MHC Class I and/or MHC Class II allele repertoire, and the resulting peptide-MHC complexes should as a second requirement be recognized by the T cells of the subject so as to induce the desired immunological reactions.

It is an object of claims of the present invention to provide improved epitope or peptide combinations for modulating an immune response, for treating a subject for an infection or aberrant immune response, and for use in diagnostic methods and kits comprising such peptide combinations, for distinguishing between latent vs active MTB infections. It is another object of the invention to provide epitope or peptide combinations exhibiting very good HLA Class I and Class II coverage in a worldwide population and being immunologically potent in a worldwide population. It is another object of the invention to provide epitope or peptide combinations having good cross-reactivity to other strains, including co-circulating strains (for example, mutants) of Mycobacterium, including MTB, etc. It is another object of the invention to provide epitope or peptide combinations of a relatively small number of epitopes or peptides yet obtaining at least 70%, and more preferably around 90-100% donor coverage in a donor cohort representative of a worldwide population. In certain embodiments, this is achieved by selecting one or more immunodominant and/or immunoprevalent proteins (e.g., a MTB protein) or subsequences, portions, homologues, variants or derivatives thereof for use in the methods and compositions of the present disclosure, wherein said immunodominant and/or immunoprevalent proteins or subsequences, portions, homologues, variants or derivatives thereof comprise two or more epitopes that are immunodominant and/or immunoprevalant. In some embodiments, the two or more epitopes comprise two to ten epitopes and/or polynucleotides encoding the same. Another object of the invention is to provide epitope combinations which are so immunologically potent that even at very low doses of epitopes, the percentage of responding donors can be retained at a very high level in a donor cohort representative of a worldwide population. Another object of the invention is to provide epitope combinations which have minor risk of inducing IgE-mediated adverse events. An additional object of the invention is to provide proteins, peptides, or nucleic acids containing or expressing epitopes or combinations of such proteins, peptides or nucleic acids which have a sufficient solubility profile for being formulated in a pharmaceutical product, preferably which have acceptable estimated in vivo stability. One further objective of the invention is to select epitopes for use in the compositions and methods described herein, based on one or both of their immunodominance or immunoprevalence. A still further object of the invention is to select such epitopes and epitopes combinations not only in accordance with those embodiments previously described, but also those epitopes and epitope combinations capable of eliciting a B cell response and T cell response (e.g., selecting one or more peptides for use in the methods and compositions described herein capable of generating a T cell and antibody response in a subject).

Provided herein are methods and compositions for diagnosing, treating, and immunizing against a Mycobacterium, including methods and compositions of detecting an immune response or immune cells relevant to a Mycobacterium infection. These methods and compositions include vaccines, diagnostics, therapies, reagents, and kits, for modulating, eliciting, or detecting T cells responsive to one or more Mycobacterium peptides or proteins. In certain embodiments, the diagnostic is capable of distinguishing between latent and active Mycobacterium infection. The proteins and peptides described herein comprise, consist of, or consist essentially of one or more amino acid sequences selected from any one of those sequences set forth in Tables 1-5 (SEQ ID NOS:1-718), or a subsequence, portion, homologue, variant, or derivative thereof; a fusion protein comprising one or more amino acid sequences selected from any one of those sequences set forth in Tables 1-5 (SEQ ID NOS:1-718); a pool of 2 or more peptides selected from the amino acid sequences set forth in any one of Tables 1-5 (SEQ ID NOS:1-718), or a polynucleotide that encodes one or more peptides or proteins, comprising, consisting of, or consisting essentially of an amino acid sequence selected from any one of those sequences set forth in Tables 1-5 (SEQ ID NOS:1-718), or a subsequence, portion, homologue, variant or derivative thereof. In certain preferred embodiments, the Mycobacterium is one or more of MTB or a variant thereof. Further description and embodiments of such methods and compositions are provided in the definitions provided herein, and a person skilled in the art will recognize that the methods and compositions can be embodied in numerous variations, changes, and substitutions or as may occur to or be understood by one skilled in the art without departing from the invention.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically, and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

What is claimed is:

1. A composition comprising:
one or more peptides or proteins, comprising, consisting of, or consisting essentially of an amino acid sequence selected from any one of those sequences set forth in Tables 1-5 (SEQ ID NOS:1-718) (SEQ ID NOS:1-718), or a subsequence, portion, homologue, variant, or derivative thereof;
a fusion protein comprising one or more amino acid sequences selected from any one of those sequences set forth in Tables 1-5 (SEQ ID NOS:1-718) (SEQ ID NOS:1-718); or
a pool of 2 or more or more peptides comprising, consisting of, or consisting essentially of amino acid sequences selected from any one of those sequences set forth in Tables 1-5 (SEQ ID NOS:1-718) (SEQ ID NOS:1-718); or
a polynucleotide that encodes one or more peptides or proteins, comprising, consisting of, or consisting essentially of an amino acid sequence selected from any one of those sequences set forth in Tables 1-5 (SEQ ID NOS:1-718) (SEQ ID NOS:1-718), or a subsequence, portion, homologue, variant, or derivative thereof.

2. The composition of claim 1, wherein the one or more peptides or proteins comprises, or wherein the fusion protein comprises 2 or more or more amino acid sequences selected from any one of those sequences set forth in Tables 1-5 (SEQ ID NOS:1-718) (SEQ ID NOS:1-718), or a subsequence, portion, homologue, variant, or derivative thereof.

3. The composition of claim 1 or claim 2, wherein the amino acid sequence is selected from a Mycobacterium T cell epitope selected from any one of those sequences set forth in Tables 1-5 (SEQ ID NOS:1-718) (SEQ ID NOS:1-718).

4. The composition of claim 1 or claim 2, wherein the composition comprises one or more MTB peptides amino acid sequences selected from any one of those sequences set forth in Tables 1-5 (SEQ ID NOS:1-718) (SEQ ID NOS:1-718), or a subsequence, portion, homologue, variant, or derivative thereof;
a fusion protein comprising one or more amino acid sequences selected from any one of those sequences set forth in Tables 1-5 (SEQ ID NOS:1-718) (SEQ ID NOS:1-718); or
a pool of 2 or more peptides selected from any one of those sequences set forth in Tables 1-5 (SEQ ID NOS:1-718)(SEQ ID NOS:1-718); or
a polynucleotide that encodes one or more peptides or proteins, comprising, consisting of, or consisting essentially of an amino acid sequence selected from any one of those sequences set forth in Tables 1-5 (SEQ ID NOS:1-718) (SEQ ID NOS:1-718), or a subsequence, portion, homologue, variant, or derivative thereof.

5. The composition of one of claims 1 to 5, wherein the peptide or protein comprises a Mycobacterium T cell epitope.

6. The composition of any one of claims 1 to 5, wherein the one or more peptides or proteins comprises a Mycobacterium CD8+ or CD4+ T cell epitope.

7. The composition of any one of claims 1 to 6, wherein the Mycobacterium is MTB and the MTB T cell epitope is not conserved in another Mycobacterium.

8. The composition of any one of claims 1 to 6, wherein the Mycobacterium is MTB and the MTB T cell epitope is conserved in another Mycobacterium.

9. The composition of any one of claims 1 to 8, wherein one or more peptides or proteins has a length from about 9-15, 15-20, 20-25, 25-30, 30-40, 40-50, 50-75 or 75-100 amino acids.

10. The composition of any one of claims 1 to 9, wherein the one or more peptides or proteins elicits, stimulates, induces, promotes, increases, or enhances a T cell response to a Mycobacterium.

11. The composition of claim 10, wherein the one or more peptides or proteins that elicits, stimulates, induces, promotes, increases, or enhances the T cell response to the Mycobacterium is a Mycobacterium protein or peptide, or a variant, homologue, derivative or subsequence thereof.

12. The composition of any one of claims 1 to 11, further comprising formulating the one or more peptides or proteins into an immunogenic formulation with an adjuvant.

13. The composition of claim 12, wherein the adjuvant is selected from the group consisting of adjuvant is selected from the group consisting of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, and TLR9 ligands.

14. The composition of any one of claims 1 to 13, wherein the composition further comprises a modulator of immune response.

15. The composition of claim 14, wherein the modulator of immune response is a modulator of the innate immune response.

16. The composition of claim 14 or claim 15, wherein the modulator is Interleukin-6 (IL-6), Interferon-gamma (IFN-γ), Transforming growth factor beta (TGF-β), or Interleukin-10 (IL-10), or an agonist or antagonist thereof.

17. A composition comprising monomers or multimers of:
peptides or proteins comprising, consisting of, or consisting essentially of:
one or more amino acid sequences selected from any one of those sequences set forth in Tables 1-5 (SEQ ID NOS:1-718) (SEQ ID NOS:1-718);
concatemers, subsequences, portions, homologues, variants, or derivatives thereof;
a fusion protein comprising one or more amino acid sequences selected from any one of those sequences set forth in Tables 1-5 (SEQ ID NOS:1-718) (SEQ ID NOS:1-718); or
a polynucleotide that encodes one or more peptides or proteins, comprising, consisting of, or consisting essentially of an amino acid sequence selected from any one of those sequences set forth in Tables 1-5 (SEQ ID NOS:1-718) (SEQ ID NOS:1-718), or a subsequence, portion, homologue, variant, or derivative thereof.

18. A composition comprising one or more peptide-major histocompatibility complex (MHC) monomers or multimers, wherein the peptide-MHC monomer or multimer comprises a peptide comprising, consisting of, or consisting essentially of an amino acid sequence selected from any one of those sequences set forth in Tables 1-5 (SEQ ID NOS:1-718) (SEQ ID NOS:1-718), in a groove of the MHC monomer or multimer.

19. A composition comprising:
one or more peptides or proteins comprising, consisting of, or consisting essentially of an amino acid sequence selected from any one of those sequences set forth in Tables 1-5 (SEQ ID NOS:1-718) (SEQ ID NOS:1-718), or a subsequence, portion, homologue, variant or derivative thereof;
a fusion protein comprising one or more amino acid sequences selected from any one of those sequences set forth in Tables 1-5 (SEQ ID NOS:1-718) (SEQ ID NOS:1-718);
a pool of 2 or more peptides selected from any one of those sequences set forth in Tables 1-5 (SEQ ID NOS:1-718) (SEQ ID NOS:1-718); or
a polynucleotide that encodes one or more peptides or proteins, comprising, consisting of, or consisting essentially of an amino acid sequence selected from any one of those sequences set forth in Tables 1-5 (SEQ ID NOS:1-718) (SEQ ID NOS:1-718), or a subsequence, portion, homologue, variant or derivative thereof.

20. The composition of claim 19, wherein the one or more peptides or proteins comprises, or wherein the fusion protein comprises, 2 or more amino acid sequences selected from any one of those sequences set forth in Tables 1-5 (SEQ ID NOS:1-718) (SEQ ID NOS:1-718), or a subsequence, portion, homologue, variant or derivative thereof.

21. The composition of claim 19 or claim 20, wherein the protein or peptide comprises a MTB T cell epitope.

22. The composition of any one of claims 19 to 21, wherein the one or more peptides or proteins comprises a MTB CD8+ or CD4+ T cell epitope.

23. The composition of any one of claims 19 to 22, wherein the MTB T cell epitope is not conserved in another Mycobacterium.

24. The composition of any one of claims 19 to 22, wherein the MTB T cell epitope is conserved in another Mycobacterium.

25. The composition of any one of claims 19 to 24, wherein one or more peptides or proteins has a length from about 9-15, 15-20, 20-25, 25-30, 30-40, 40-50, 50-75 or 75-100 amino acids.

26. The composition of any one of claims 19 to 25, wherein the one or more peptides or proteins elicits, stimulates, induces, promotes, increases or enhances a T cell response to MTB.

27. The composition of any one of claims 19 to 26, wherein the one or more peptides or proteins that elicits, stimulates, induces, promotes, increases or enhances the T cell response to MTB is a MTB protein or peptide, or a variant, homologue, derivative or subsequence thereof.

28. The composition of any one of claims 19 to 27, further comprising formulating the one or more peptides or proteins into an immunogenic formulation with an adjuvant.

29. The composition of claim 28, wherein the adjuvant is selected from the group consisting of adjuvant is selected from the group consisting of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, and TLR9 ligands.

30. The composition of any one of claims 19 to 29, wherein the composition further comprises a modulator of immune response.

31. The composition of claim 30, wherein the modulator of immune response is a modulator of the innate immune response.

32. The composition of claim 30 or claim 31, wherein the modulator is Interleukin-6 (IL-6), Interferon-gamma (IFN-g), Transforming growth factor beta (TGF-B), or Interleukin-10 (IL-10), or an agonist or antagonist thereof.

33. A composition comprising monomers or multimers of:
one or more peptides or proteins comprising, consisting of, or consisting essentially of:
one or more MTB amino acid sequences selected from any one of those sequences set forth in Tables 1-5 (SEQ ID NOS:1-718) (SEQ ID NOS:1-718), concatemers, subsequences, portions, homologues, variants or derivatives thereof;
a fusion protein comprising one or more amino acid sequences selected from any one of those sequences set forth in Tables 1-5 (SEQ ID NOS:1-718) (SEQ ID NOS:1-718); or
a polynucleotide that encodes one or more peptides or proteins, comprising, consisting of, or consisting essentially of an amino acid sequence selected from any one of those sequences set forth in Tables 1-5 (SEQ ID NOS:1-718) (SEQ ID NOS:1-718), or a subsequence, portion, homologue, variant or derivative thereof.

34. A composition comprising one or more peptide-major histocompatibility complex (MHC) monomers or multimers, wherein the peptide-MHC monomer or multimer comprises a peptide comprising, consisting of, or consisting essentially of an amino acid sequence selected from any one of those sequences set forth in Tables 1-5 (SEQ ID NOS:1-718) (SEQ ID NOS:1-718), in a groove of the (MHC) monomer or multimer.

35. A method for detecting the presence of: (i) a Mycobacterium or (ii) an immune response relevant to Mycobacterium infections, vaccines or therapies, including T cells responsive to one or more Mycobacterium peptides, comprising:
providing one or more proteins or peptides for detection of an amount or a relative amount of, and/or the activity of, and/or the state of antigen-specific T-cells;
contacting a biological sample suspected of having Mycobacterium-specific T-cells to one or more proteins or peptides for detection; and
detecting an amount or a relative amount of, and/or the activity of, and/or the state of antigen-specific T-cells in the biological sample, wherein the one or more proteins or peptides for detection comprise one or more amino acid sequences set forth in any one of Tables 1-5 (SEQ ID NOS:1-718), or comprise a pool of 2 or more or more amino acid sequences set forth in any one of Tables 1-5 (SEQ ID NOS:1-718).

36. The method of claim 35, wherein detecting the amount or a relative amount of, and/or activity of antigen-specific T-cells comprises one or more steps of identification or detection of the antigen-specific T-cells and measuring the amount of the antigen-specific T-cells.

37. The method of claim 35 or claim 36, wherein the one or more peptides or proteins comprises 2 or more amino acid sequences selected from those set forth in any one of Tables 1-5 (SEQ ID NOS:1-718).

38. The method of any one of claims 35 to 37, wherein the detecting the amount or a relative amount of, and/or activity of antigen-specific T-cells comprises indirect detection and/or direct detection.

39. The method of any one of claims 35 to 38, wherein the method of detecting an immune response relevant to the Mycobacterium comprises the following steps:
providing an MHC monomer or an MHC multimer;
contacting a population T-cells to the MHC monomer or MHC multimer; and
measuring the number, activity or state of T-cells specific for the MHC monomer or MHC multimer.

40. The method of claim 39, wherein the MHC monomer or MHC multimer comprises a protein or peptide of the Mycobacterium.

41. The method of claim 35, wherein the protein or peptide comprises a CD8+ or CD4+ T cell epitope.

42. The method of claim 41, wherein the T cell epitope is not conserved in another Mycobacterium.

43. The method of claim 41, wherein the T cell epitope is conserved in another Mycobacterium.

44. The method of any one of claims 35 to 43, wherein the protein or peptide has a length from about 9-15, 15-20, 20-25, 25-30, 30-40, 40-50, 50-75 or 75-100 amino acids.

45. The method of any one of claims 35 to 44, wherein the proteins or peptides comprise 2 or more amino acid sequences selected from any one of those sequences set forth in Tables 1-5 (SEQ ID NOS:1-718), or a subsequence, portion, homologue, variant or derivative thereof.

46. The method of any one of claims 35 to 45, further comprising detecting the presence or amount of the one or more peptides in a biological sample, or a response thereto, which is diagnostic of a latent or active Mycobacterium infection.

47. The method of any one of claims 35 to 46, wherein detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay.

48. The method of any one of claims 35 to 47, further comprising administering a treatment comprising the composition of any one of claims 1-36 to the subject from which the biological sample was drawn that increases the amount or relative amount of, and/or activity of the antigen-specific T-cells.

49. A method for detecting the presence of: (i) MTB or (ii) an immune response relevant to MTB infections, vaccines or therapies, including T cells responsive to one or more MTB peptides, comprising:
providing one or more proteins or peptides for detection of an amount or a relative amount of, and/or the activity of, and/or the state of antigen-specific T-cells;
contacting a biological sample suspected of having MTB-specific T-cells to one or more proteins or peptides for detection; and
detecting an amount or a relative amount of, and/or the activity of, and/or the state of antigen-specific T-cells in the biological sample, wherein the one or more proteins or peptides for detection comprise one or more amino acid sequences set forth in those sequences set forth in any one of Tables 1-5 (SEQ ID NOS:1-718), or comprise a pool of 2 or more amino acid sequences set forth in those sequences set forth in any one of Tables 1-5 (SEQ ID NOS:1-718).

50. The method of claim 49, wherein detecting the amount or a relative amount of, and/or activity of antigen-specific T-cells comprises one or more steps of identification or detection of the antigen-specific T-cells and measuring the amount of the antigen-specific T-cells.

51. The method of claim 49 or claim 50, wherein the one or more peptides or proteins comprises 2 or more amino acid sequences selected from any one of those sequences set forth in Tables 1-5 (SEQ ID NOS:1-718).

52. The method of any one of claims 49 to 51, wherein the detecting the amount or a relative amount of, and/or activity of antigen-specific T-cells comprises indirect detection and/or direct detection.

53. The method of any one of claims 49 to 52, wherein the method of detecting an immune response relevant to MTB comprises the following steps:
providing an MHC monomer or an MHC multimer;
contacting a population T-cells to the MHC monomer or MHC multimer; and
measuring the number, activity or state of T-cells specific for the MHC monomer or MHC multimer.

54. The method of claim 53, wherein the MHC monomer or MHC multimer comprises a protein or peptide of MTB.

55. The method of claim 54, wherein the protein or peptide comprises a MTB CD8+ or CD4+ T cell epitope.

56. The method of claim 55, wherein the MTB T cell epitope is not conserved in another Mycobacterium.

57. The method of claim 55, wherein the MTB T cell epitope is conserved in another Mycobacterium.

58. The method of any one of claims 49 to 57, wherein the protein or peptide has a length from about 9-15, 15-20, 20-25, 25-30, 30-40, 40-50, 50-75 or 75-100 amino acids.

59. The method of any one of claims 49 to 58, wherein the proteins or peptides comprise 2 or more amino acid sequences selected from any one of those sequences set forth in Tables 1-5 (SEQ ID NOS:1-718), or a subsequence, portion, homologue, variant or derivative thereof.

60. The method of any one of claims 49 to 59, further comprising detecting the presence or amount of the one or more peptides in a biological sample, or a response thereto, which is diagnostic of a latent or active MTB infection.

61. The method of any one of claims 49 to 60, wherein detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay.

62. The method of any one of claims 49 to 61, further comprising administering a treatment comprising the composition of any one of claims 1-34 to the subject from which the biological sample was drawn that increases the amount or relative amount of, and/or activity of the antigen-specific T-cells.

63. A method detecting a Mycobacterium infection or exposure in a subject, the method comprising, consisting of, or consisting essentially of:
contacting a biological sample from a subject with a composition of any one of claims 1 to 36; and
determining if the composition elicits an immune response from the contacted cells, wherein the presence of an immune response indicates that the subject has been exposed to or infected with Mycobacterium.

64. The method of claim 63, wherein the sample comprises T cells.

65. The method of claim 63 or claim 64, wherein the response comprises inducing, increasing, promoting or stimulating anti-Mycobacterium activity of T cells.

66. The method of claim 64 or claim 65, wherein the T cells are CD8+ or CD4+ T cells.

67. The method of any one of claims 63 to 66, wherein the method comprises determining whether the subject has been infected by or exposed to the Mycobacterium more than once by determining if the subject elicits a secondary T cell immune response profile that is different from a primary T cell immune response profile.

68. The method of any one of claims 63 to 67, further comprising diagnosing a latent or active Mycobacterium infection or exposure in a subject, the method comprising contacting a biological sample from a subject with a composition of any one of claims 1 to 36, and determining if the composition elicits a T cell immune response, wherein the T cell immune response identifies that the subject has been infected with or exposed to a Mycobacterium.

69. The method of any one of claims 63 to 68, wherein the method is conducted three or more days following the date of suspected infection by or exposure to a Mycobacterium.

70. A method detecting MTB infection or exposure in a subject, the method comprising, consisting of, or consisting essentially of:
contacting a biological sample from a subject with a composition of any one of claims 19 to 34; and
determining if the composition elicits an immune response from the contacted cells, wherein the presence of an immune response indicates that the subject has been exposed to or infected with MTB.

71. The method of claim 70, wherein the sample comprises T cells.

72. The method of claim 70 or claim 71, wherein the response comprises inducing, increasing, promoting or stimulating anti-MTB activity of T cells.

73. The method of claim 71 or claim 72, wherein the T cells are CD8+ or CD4+ T cells.

74. The method of any one of claims 70 to 73, wherein the method comprises determining whether the subject has been infected by or exposed to MTB more than once by determining if the subject elicits a secondary T cell immune response profile that is different from a primary T cell immune response profile.

75. The method of any one of claims 70 to 74, further comprising diagnosing a latent or active MTB infection or exposure in a subject, the method comprising contacting a biological sample from a subject with a composition of any one of claims 19 to 36; and determining if the composition elicits a T cell immune response, wherein the T cell immune response identifies that the subject has been infected with or exposed to MTB.

76. The method of any one of claims 70 to 75, wherein the method is conducted three or more days following the date of suspected infection by or exposure to a Mycobacterium.

77. A kit for the detection of Mycobacterium or an immune response to Mycobacterium in a subject comprising, consisting of or consisting essentially of:
one or more T cells that specifically detect the presence of:
one or more amino acid sequences selected from any one of those sequences set forth in Tables 1-5 (SEQ ID NOS:1-718), or a subsequence, portion, homologue, variant or derivative thereof; or
a fusion protein comprising one or more amino acid sequences selected from any one of those sequences set forth in Tables 1-5 (SEQ ID NOS:1-718); or
a pool of 2 or more or more peptides selected from the amino acid sequences set forth in any one of Tables 1-5 (SEQ ID NOS:1-718).

78. The kit of claim 77, wherein the one or more amino acid sequences are selected from a Mycobacterium T cell epitope set forth in any one of Tables 1-5 (SEQ ID NOS:1-718).

79. The kit of claim 77 or claim 78, wherein the composition comprises:
one or more amino acid sequences selected from any one of those sequences set forth in Tables 1-5 (SEQ ID NOS:1-718), or a subsequence, portion, homologue, variant or derivative thereof;
a fusion protein comprising one or more amino acid sequences selected from any one of those sequences set forth in Tables 1-5 (SEQ ID NOS:1-718); or
a pool of 2 or more peptides selected from the amino acid sequences set forth in those sequences set forth in any one of Tables 1-5 (SEQ ID NOS:1-718).

80. The kit of any one of claims 77 to 79, wherein the amino acid sequence comprises a Mycobacterium CD8+ or CD4+ T cell epitope.

81. The kit of claim 78 or claim 80, wherein the T cell epitope is not conserved in another Mycobacterium.

82. The kit of claim 78 or claim 80, wherein the T cell epitope is conserved in another Mycobacterium.

83. The kit of any one of claims 77 to 82, wherein the fusion protein has a length from about 9-15, 15-20, 20-25, 25-30, 30-40, 40-50, 50-75 or 75-100 amino acids.

84. The kit of any one of claims 77 to 83, wherein the kit includes instruction for a diagnostic method, a process, a composition, a product, a service or component part thereof for the detection of: (i) Mycobacterium or (ii) an immune response relevant to Mycobacterium infections, vaccines or therapies, including T cells responsive to Mycobacterium.

85. The kit of any one of claims 77 to 84, wherein the kit includes reagents for detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay.

86. The kit of any one of claims 77 to 85, wherein the kit includes reagents for determining a Human Leukocyte Antigen (HLA) profile of a subject, and selecting peptides that are presented by the HLA profile of the subject for detecting an immune response to Mycobacterium.

87. A kit for the detection of MTB or an immune response to MTB in a subject comprising, consisting of or consisting essentially of:
one or more T cells that specifically detect the presence of:
one or more amino acid sequences selected from any one of those sequences set forth in Tables 1-5 (SEQ ID NOS:1-718), or a subsequence, portion, homologue, variant or derivative thereof;
a fusion protein comprising one or more amino acid sequences selected from any one of those sequences set forth in Tables 1-5 (SEQ ID NOS:1-718); or
a pool of 2 or more peptides selected from the amino acid sequences set forth in those sequences set forth in any one of Tables 1-5 (SEQ ID NOS:1-718).

88. The kit of claim 87, wherein the one or more amino acid sequences is selected from a MTB CD4 T cell epitope selected from any one of Tables 1-5 (SEQ ID NOS:1-718); or both.

89. N/A.

90. The kit of claims 87 to 88, wherein the amino acid sequence comprises a MTB CD8+ or CD4+ T cell epitope.

91. The kit of claim 90, wherein the MTB T cell epitope is not conserved in another Mycobacterium.

92. The kit of claim 90, wherein the MTB T cell epitope is conserved in another Mycobacterium.

93. The kit of any one of claims 87 to 92, wherein the fusion protein has a length from about 9-15, 15-20, 20-25, 25-30, 30-40, 40-50, 50-75 or 75-100 amino acids.

94. The kit of any one of claims 87 to 93, wherein the kit includes instruction for a diagnostic method, a process, a composition, a product, a service or component part thereof for the detection of: (i) MTB or (ii) an immune response relevant to MTB infections, vaccines or therapies, including T cells responsive to MTB.

95. The kit of any one of claims 87 to 94, wherein the kit includes reagents for detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay.

96. The kit of any one of claims 87 to 95, wherein the kit includes reagents for determining a Human Leukocyte Antigen (HLA) profile of a subject, and selecting peptides that are presented by the HLA profile of the subject for detecting an immune response to MTB.

97. A method of stimulating, inducing, promoting, increasing, or enhancing an immune response against a Mycobacterium in a subject, comprising:
administering a composition of claims 1 to 34, in an amount sufficient to stimulate, induce, promote, increase, or enhance an immune response against the Mycobacterium in the subject.

98. The method of claim 97, wherein the immune response provides the subject with protection against a Mycobacterium infection or pathology, or one or more physiological conditions, disorders, illnesses, diseases or symptoms caused by or associated with Mycobacterium infection or pathology.

99. The method of claim 97 or claim 98, wherein the immune response is specific to:
one or more MTB peptides selected from the amino acid sequences set forth in any one of Tables 1-5 (SEQ ID NOS:1-718), or a subsequence, portion, homologue, variant or derivative thereof.

100. A method of stimulating, inducing, promoting, increasing, or enhancing an immune response against MTB in a subject, comprising:
administering a composition of claims to 19 to 34, in an amount sufficient to stimulate, induce, promote, increase, or enhance an immune response against MTB in the subject.

101. The method of claim 100, wherein the immune response provides the subject with protection against a MTB infection or pathology, or one or more physiological conditions, disorders, illnesses, diseases or symptoms caused by or associated with MTB infection or pathology.

102. The method of claim 100 or claim 101, wherein the immune response is specific to:
one or more MTB peptides selected from the amino acid sequences set forth in those sequences set forth in any one of Tables 1-5 (SEQ ID NOS:1-718), or a subsequence, portion, homologue, variant or derivative thereof.

103. A method of stimulating, inducing, promoting, increasing, or enhancing an immune response against MTB in a subject, comprising:
administering to a subject an amount of a protein or peptide or a polynucleotide that expresses the protein or peptide comprising, consisting of or consisting essentially of an amino acid sequence of the MTB protein or peptide, or a variant, homologue, derivative or subsequence thereof, wherein the protein or peptide comprises at least two peptides selected from the amino acid sequences set forth in any one of Tables 1-5 (SEQ ID NOS:1-718) or a subsequence, portion, homologue, variant or derivative thereof, in an amount sufficient to prevent, stimulate, induce, promote, increase, immunize against, or enhance an immune response against MTB in the subject.

104. The method of claim 103, wherein the immune response provides the subject with protection against MTB infection or pathology, or one or more physiological conditions, disorders, illnesses, diseases or symptoms caused by or associated with MTB infection or pathology.

105. A method of treating, preventing, or immunizing a subject against MTB infection, comprising administering to a subject an amount of a protein, peptide or a polynucleotide that expresses the protein or peptide comprising, consisting of, or consisting essentially of an amino acid sequence of a Mycobacterium protein or peptide, or a variant, homologue, derivative or subsequence thereof, wherein the protein or peptide comprises at least two amino acid sequences selected from any one of Tables 1-5 (SEQ ID NOS:1-718) or a subsequence, portion, homologue, variant or derivative thereof, in an amount sufficient to treat, prevent, or immunize the subject for MTB infection, wherein the protein or peptide comprises or consists of a Mycobacterium T cell epitope that elicits, stimulates, induces, promotes, increases, or enhances an anti-MTB T cell immune response.

106. The method of claim 105, wherein the one or more amino acid sequences are selected from any one of those sequences set forth in Tables 1-5 (SEQ ID NOS:1-718), or a subsequence, portion, homologue, variant or derivative thereof;
a fusion protein comprising one or more amino acid sequences selected from any one of those sequences set forth in Tables 1-5 (SEQ ID NOS:1-718); or
a pool of 2 or more peptides selected from the amino acid sequences set forth in those sequences set forth in Tables 1-5 (SEQ ID NOS:1-718).

107. The method of claim 105, wherein the anti-MTB T cell response is a CD8+, a CD4+ T cell response, or both.

108. The method of any of claims 105 to 107, wherein the T cell epitope is conserved across two or more clinical isolates of MTB or two or more circulating forms of MTB.

109. The method of claim 108, wherein the MTB infection is an acute infection.

110. The method of any one of claims 105 to 109, wherein the subject is a mammal or a human.

111. The method of any one of claims 105 to 110, wherein the method reduces MTB bacterial titer, increases or stimulates MTB bacterial clearance, reduces or inhibits MTB bacterial proliferation, reduces or inhibits increases in MTB bacterial titer or MTB bacterial proliferation, reduces the amount of a MTB bacterial protein or the amount of a MTB bacterial nucleic acid, or reduces or inhibits synthesis of a MTB bacterial protein or a MTB bacterial nucleic acid.

112. The method of any one of claims 105 to 111, wherein the method reduces one or more adverse physiological conditions, disorders, illness, diseases, symptoms or complications caused by or associated with MTB infection or pathology.

113. The method of any one of claims 105 to 112, wherein the method improves one or more adverse physiological conditions, disorders, illness, diseases, symptoms or complications caused by or associated with MTB infection or pathology.

114. The method of claim 112 or 113, wherein the symptom is fever or chills, cough, shortness of breath or difficulty breathing, fatigue, muscle or body aches, headache, new loss of taste or smell, sore throat, congestion or runny nose, nausea or vomiting, or diarrhea.

115. The method of any one of claims 105 to 114, wherein the method reduces or inhibits susceptibility to MTB infection or pathology.

116. The method of any one of claims 105 to 115, wherein the protein or peptide, or a subsequence, portion, homologue, variant or derivative thereof, is administered prior to, substantially contemporaneously with or following exposure to or infection of the subject with MTB.

117. The method of any one of claims 105 to 116, wherein a plurality of MTB T cell epitopes are administered prior to, substantially contemporaneously with or following exposure to or infection of the subject with MTB.

118. The method of any one of claims 105 to 117, wherein the protein or peptide, or a subsequence, portion, homologue, variant or derivative thereof is administered within 2-72 hours, 2-48 hours, 4-24 hours, 4-18 hours, or 6-12 hours after a symptom of MTB infection or exposure develops.

119. The method of any one of claims 105 to 118, wherein the protein or peptide, or a subsequence, portion, homologue, variant or derivative thereof is administered prior to exposure to or infection of the subject with MTB.

120. The method of any one of claims 105 to 118, wherein the method further comprises administering a modulator of immune response prior to, substantially contemporaneously with or following the administration to the subject of an amount of a protein or peptide.

121. The method of claim 120, wherein the modulator of immune response is a modulator of the innate immune response.

122. The method of claim 120 or claim 121, wherein the modulator is IL-6, IFN-γ, TGF-β, or IL-10, or an agonist or antagonist thereof.

123. A method of treating, preventing, or immunizing a subject against MTB infection, comprising administering to a subject the composition of any one of claims 1-36 in an amount sufficient to treat, prevent, or immunize the subject for MTB infection.

124. The method of claim 123, wherein the MTB infection is an acute infection.

125. The method of claim 123, wherein the method reduces MTB bacterial titer, increases or stimulates MTB bacterial clearance, reduces or inhibits MTB bacterial proliferation, reduces or inhibits increases in MTB bacterial titer or MTB bacterial proliferation, reduces the amount of a MTB bacterial protein or the amount of a MTB bacterial nucleic acid, or reduces or inhibits synthesis of a MTB bacterial protein or a MTB bacterial nucleic acid.

126. The method of any one of claims 123 to 125, wherein the method reduces one or more adverse physiological conditions, disorders, illness, diseases, symptoms or complications caused by or associated with MTB infection or pathology.

127. The method of any one of claims 123 to 126, wherein the method improves one or more adverse physiological conditions, disorders, illness, diseases, symptoms or complications caused by or associated with MTB infection or pathology.

128. The method of claim 126 or claim 127, wherein the symptom is fever or chills, cough, shortness of breath or difficulty breathing, fatigue, muscle or body aches, headache, new loss of taste or smell, sore throat, congestion or runny nose, nausea, vomiting, or diarrhea.

129. The method of any one of claims 123 to 128, wherein the method reduces or inhibits susceptibility to MTB infection or pathology.

130. The method of any one of claims 123 to 129, wherein the composition is administered prior to, substantially contemporaneously with or following exposure to or infection of the subject with MTB.

131. The method of any one of claims 123 to 130, wherein the composition is administered prior to, substantially contemporaneously with or following exposure to or infection of the subject with MTB.

132. The method of any one of claims 123 to 131, wherein the composition is administered within 2-72 hours, 2-48 hours, 4-24 hours, 4-18 hours, or 6-12 hours after a symptom of MTB infection or exposure develops.

133. The method of any one of claims 123 to 131, wherein the composition is administered prior to exposure to or infection of the subject with MTB.

134. A peptide or peptides that are immunoprevalent or immunodominant in a bacteria obtained by a method consisting of, or consisting essentially of:
obtaining an amino acid sequence of the bacteria;
determining one or more sets of overlapping peptides spanning one or more bacteria antigen using unbiased selection;
synthesizing one or more pools of bacteria peptides comprising the one or more sets of overlapping peptides;
combining the one or more pools of bacteria peptides with Class I major histocompatibility proteins (MHC), Class II MHC, or both Class I and Class II MHC to form peptide-MHC complexes;
contacting the peptide-MHC complexes with T cells from subjects exposed to the bacteria;
determining which pools triggered cytokine release by the T cells; and
deconvoluting from the pool of peptides that elicited cytokine release by the T cells, which peptide or peptides are immunoprevalent or immunodominant in the pool.

135. The peptide or peptides of claim 134, wherein the bacteria is a Mycobacterium.

136. The peptide or peptides of claim 135, wherein the Mycobacterium is MTB.

137. The peptide or peptides of any one of claims 134 to 136, wherein the immunodominant peptides are selected from 1, 2 or more peptides selected from the amino acid sequences set forth in any one of Tables 1-5 (SEQ ID NOS:1-718).

138. The peptide or peptides of any one of claims 134 to 137, wherein the immunodominant peptides are selected from 1, 2 or more peptides selected from the amino acid sequences set forth in those sequences set forth in any one of Tables 1-5 (SEQ ID NOS:1-718).

139. A method of selecting an immunoprevalent or immunodominant peptide or protein of a bacteria comprising, consisting of, or consisting essentially of:
obtaining an amino acid sequence of the bacteria;
determining one or more sets of overlapping peptides spanning one or more bacteria antigen using unbiased selection;
synthesizing one or more pools of bacteria peptides comprising the one or more sets of overlapping peptides;
combining the one or more pools of bacteria peptides with Class I major histocompatibility proteins (MHC), Class II MHC, or both Class I and Class II MHC to form peptide-MHC complexes;
contacting the peptide-MHC complexes with T cells from subjects exposed to the bacteria;
determining which pools triggered cytokine release by the T cells; and
deconvoluting from the pool of peptides that elicited cytokine release by the T cells, which peptide or peptides are immunoprevalent or immunodominant in the pool.

140. The method of claim 139, wherein the bacteria is a Mycobacterium.

141. The method of claim 140, wherein the Mycobacterium is MTB.

142. The method of any one of claim 139 to 141, wherein the immunodominant peptides are selected from 1, 2 or more peptides selected from the amino acid sequences set forth in any one of Tables 1-5 (SEQ ID NOS:1-718).

143. The method of any one of claims 139 to 142, wherein the immunodominant peptides are selected from 1, 2 or more peptides selected from the amino acid sequences set forth in those sequences set forth in any one of Tables 1-5 (SEQ ID NOS:1-718).

144. A polynucleotide that expresses one or more peptides or proteins, comprising, consisting of, or consisting essentially of an amino acid sequence selected from any one of those sequences set forth in Tables 1-5 (SEQ ID NOS:1-718), or a subsequence, portion, homologue, variant or derivative thereof;
a fusion protein comprising one or more amino acid sequences selected from any one of those sequences set forth in Tables 1-5 (SEQ ID NOS:1-718); or
a pool of 2 or more or more peptides comprising, consisting of, or consisting essentially of amino acid sequences selected from any one of those sequences set forth in Tables 1-5 (SEQ ID NOS:1-718).

145. A vector that comprises the polynucleotide of claim 144.

146. The vector of claim 145, wherein the vector is a bacterial vector.

147. A host cell that comprises the vector of claim 145 or claim 146.

148. A polynucleotide that expresses:
one or more peptides or proteins comprising, consisting of, or consisting essentially of an amino acid sequence selected from any one of those sequences set forth in Tables 1-5 (SEQ ID NOS:1-718), or a subsequence, portion, homologue, variant or derivative thereof;
a fusion protein comprising one or more amino acid sequences selected from any one of those sequences set forth in Tables 1-5 (SEQ ID NOS:1-718); or
a pool of 2 or more peptides selected from any one of those sequences set forth in Tables 1-5 (SEQ ID NOS:1-718).

149. A vector that comprises the polynucleotide of claim 148.

150. The vector of claim 149, wherein the vector is a bacterial vector.

151. A host cell that comprises the vector of claim 149 or claim 150.
